(19) United States
(12) Patent Application Publication
(10) Pub. No.: US 2025/0289806 A1
(43) Pub. Date: Sep. 18, 2025

(54) COMPOUNDS FOR PROLIFERATIVE DISORDERS

(71) Applicant: UNIVERSITY OF MIAMI, Miami, FL (US)

(72) Inventors: Yangbo FENG, Miami, FL (US); Justin TAYLOR, Miami, FL (US); Sana CHAUDHRY, Miami, FL (US)

(21) Appl. No.: 18/861,063

(22) PCT Filed: Apr. 27, 2023
(86) PCT No.: PCT/US2023/020177

§ 371 Date: Oct. 28, 2024

Related U.S. Application Data
(60) Provisional application No. 63/335,951, filed on Apr. 28, 2022.

(51) Int. Cl. C07D 403/14; A61K 31/517; A61K 31/5377; A61K 31/5386; C07D 239/84; C07D 401/14; C07D 405/14; C07D 417/14; C07D 471/04; C07D 471/14; C07D 487/08; C07D 491/147; C07D 498/08; C07D 519/00

(52) U.S. Cl. CPC C07D 403/14; A61K 31/517; A61K 31/5377; A61K 31/5386; C07D 239/84; C07D 401/14; C07D 405/14; C07D 417/14; C07D 471/04; C07D 471/14; C07D 487/08; C07D 491/147; C07D 498/08; C07D 519/00

(57) ABSTRACT

Disclosed herein are novel compounds with STK17A inhibitory activity. The compounds may be used to treat proliferative disorders, including myelodysplastic syndrome and leukemia.

### CROSS-REFERENCE TO RELATED APPLICATION

This application claims the benefit of U.S. Provisional Application 63/335,951, filed Apr. 28, 2022, the contents of which are hereby incorporated in its entirety.

### FIELD OF THE INVENTION

The invention is directed, in part, to novel compounds with STK17A inhibitory activity. The compounds may be used to treat proliferative disorders, including myelodysplastic syndrome, leukemia, and glioblastoma.

### BACKGROUND

Myelodysplastic syndromes (MDS) represent a spectrum of myeloid neoplasms involving clonal disorders of hematopoietic stem and progenitor cells (HSPC), varying degrees of cytopenias, dysplasia, and variable clinical course, with the feared risk of transformation into acute myeloid leukemia (AML). The incidence of MDS in the U.S. is 4.9 per 100,000 people per year. Recurrent gain-of-function mutations in RNA splicing factors as a group are the most highly prevalent class of mutations in myelodysplastic syndromes (MDS) and represent clear drivers of disease. Mutations in SF3B1 occur as heterozygous hotspot mutations and are the most frequently mutated splicing gene in MDS.

Currently, there are no approved therapies that target spliceosomal mutations in any cancer; thus, there is an urgent unmet medical need to develop therapies that could be used in SF3B1-mutated myeloid malignancies including SF3B1 mutant MDS. The current FDA approved standard treatments for myelodysplastic syndrome are lenalidomide, luspatercept and hypomethylating agents (azacitidine and decitabine). Lenalidomide is an immunomodulatory drug (IMiD) with efficacy in the subset of MDS known as deletion 5q MDS. Luspatercept is a recombinant fusion protein that binds TGF-β superfamily ligands and causes erythroid maturation. Luspatercept was recently approved to treat anemia in patients with lower-risk MDS and is typically used for those that do not respond to erythropoietin-stimulating agents. Therefore, the only disease-modifying treatments for patients with MDS with other cytopenias besides anemia, or with high-risk disease are the hypomethylating agents. Azacitidine is administered as an injection or infusion in continuous cycles until disease progression.

Decitabine is given as an infusion but recently a fixed dose combination of decitabine and cedazurine in an oral tablet was approved based on bioequivalence to intravenous decitabine. Finally, there is no standard of care treatment for patients who progress on hypomethylating agents. Recent clinical trials attempting to improve standard of care by adding a second drug to hypomethylating agents have failed. These doublets, such as eprenetapopt or pevonedistat added to azacitidine, have been compared to azacitidine alone in randomized phase 3 trials that failed to meet their primary endpoint. Other drugs being evaluated in MDS are magrolimab, a form of immunotherapy, and venetoclax, a pro-apoptotic drug. Nevertheless there remains a need for improved STK17A inhibitors. There remains a need for selective STK17A inhibitors. There remains a need for improved therapies for the treatment of proliferative disorders, including cancers and myelodysplastic syndrome. The remains a need for improved therapies for glioblastoma. There remains a need for improved therapies for leukemia, including acute myeloid leukemia.

In accordance with the purposes of the disclosed materials and methods, as embodied and broadly described herein, the disclosed subject matter, in one aspect, relates to compounds, compositions and methods of making and using compounds and compositions.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

The details of one or more embodiments are set forth in the descriptions below. Other features, objects, and advantages will be apparent from the description and from the claims.

### BRIEF DESCRIPTION OF THE FIGURES

FIG. 1 depicts in vivo pharmacokinetics of Example 7 following intraperitoneal and oral administration. Administration of 10 mg/kg of Example 7 to 4 mice with median and standard deviation displayed at timepoints of plasma collection and measurement by mass spectrometry.

FIG. 2 depicts complete metabolic panel results from mice treated with vehicle (control) or increasing doses of Example 7. Mice were dosed with 30, 100 or 300 mg/kg of Example 7 and blood drawn 24 hours

FIG. 3 depicts the increased STK17A expression in SF3B1 mutant cells. Triplicate results by qPCR in isogenic K562 cells.

FIG. 4 depicts the half-maximal inhibitory concentrations (IC₅₀) determined for Example 7 with the use of cell viability assays at 72 hours of various cancer cell lines compared to a non-cancer cell line.

FIG. 5A depicts tumor volume of isogenic K562 cell line xenograft followed by administration of 20 mg/kg of vehicle and Example 7 to 5 mice each.

FIG. 5B depicts a survival curve of isogenic K562 cell line xenograft followed by administration of 20 mg/kg of vehicle and Example 7 to 5 mice each.

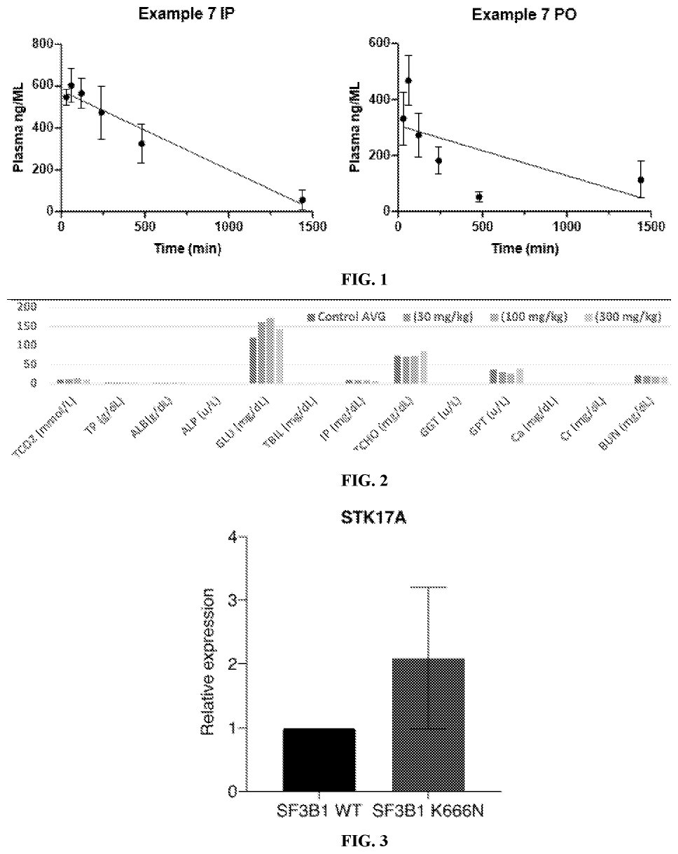

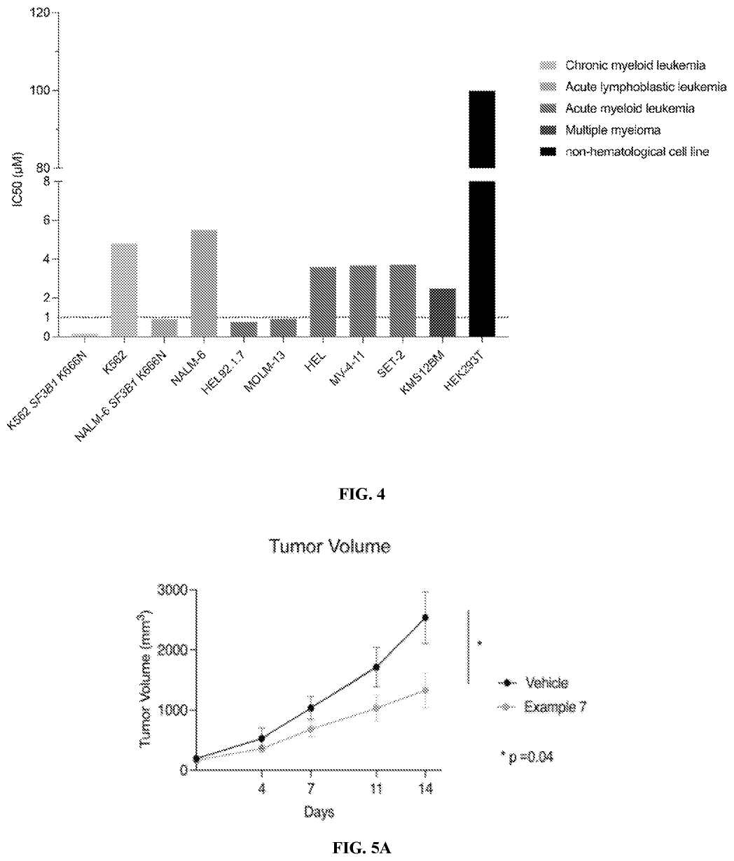

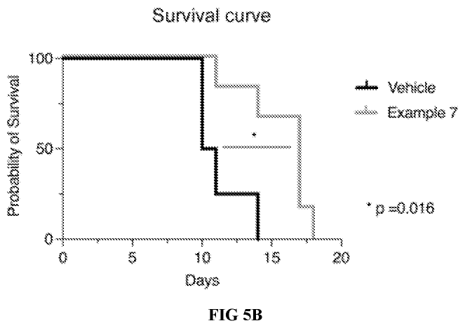

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes-from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions, Wiley Interscience, New York, 1981; Wilen et al., Tetrahedron 33:2725 (1977); Eliel, E. L. Stereochemistry of Carbon Compounds, McGraw-Hill, NY, 1962; and Wilen, S. H., Tables of Resolving Agents and Optical Resolutions p. 268, E. L. Eliel, Ed., Univ. of Notre Dame Press, Notre Dame, IN 1972. The invention additionally encompasses compounds as individual isomers substantially free of other isomers, and alternatively, as mixtures of various isomers.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched hydrocarbon group having a specified range of carbon atoms (e.g., a “C₁₋₁₆ alkyl” can have from 1 to 16 carbon atoms). In some embodiments, an alkyl group has 1 to 9 carbon atoms (“C₁₋₉ alkyl”). An alkyl group can be saturated or unsaturated, i.e., an alkenyl or alkynyl group as defined herein. Unless specified to the contrary, an “alkyl” group includes both saturated alkyl groups and unsaturated alkyl groups. Unless explicitly specified to the contrary, “alkyl” is not limited to monovalent species. When an alkyl group appears in a chemical context that requires a polyvalent (e.g., two, three, or four points of attachment), it is to be understood that the valency of the alkyl group satisfies the number of attachment points. The same non-limiting definition applies to other groups, e.g., aryl, heteroaryl, heterocyclyl, carbocyclyl, etc.

The term “alkoxy” refers to an alkyl group, as defined herein, appended to the parent molecular moiety through an oxygen atom.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋₈ alkoxyalkyl”).

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain.

The term “carbocyclyl,” “cycloalkyl,” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. Exemplary C₃₋₆ carbocyclyl groups include, without limitation, cyclopropyl (C₃), cyclopropenyl (C₃), cyclobutyl (C₄), cyclobutenyl (C₄), cyclopentyl (Cs), cyclopentenyl (Cs), cyclohexyl (C₆), cyclohexenyl (C₆), cyclohexadienyl (C₆), and the like.

As used herein, the term “heterocyclyl” refers to an aromatic (also referred to as a heteroaryl), unsaturated, or saturated cyclic hydrocarbon that includes at least one heteroatom in the cycle. For example, the term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or more rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

The term “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). Unless otherwise specified, each instance of an aryl group is independently unsubstituted (an “unsubstituted aryl”) or substituted (a “substituted aryl”) with one or more substituents.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, -Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃X⁻, —P(OR^{cc})₃⁺X⁻, —P(R^{cc})₄, —P(OR^{cc})₂, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; or two geminal hydrogens on a carbon atom are replaced with the group ═O, ═S, ═NN(R^{bb})₂, ═NNR^{bb}C(═O)R^{aa}, =NNR^{bb}C(═O)OR^{aa}, =NNR^{bb}S(═O)₂R^{aa}, =NR^{bb} or =NOR^{cc}; each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(O)N(R^{ff})₂, —C(═NR^{ff})OR^{ee}, —OC(═NR)R^{ee}, —OC(═NR)OR^{ee}, —C(═NR^{ff})N(R^{ff})₂, —OC(═NR^{ff})N(R^{ff})₂, —NR^{ff}C(═NR^{ff})N(R^{K})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R⁹⁹ groups, or two geminal R^{dd} substituents can be joined to form ═O or ═S; wherein X⁻ is a counterion; each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{f} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)*X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃, —C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “oxo” refers to the group ═O, and the term “thiooxo” refers to the group ═S.

As used herein, a chemical bond depicted: [symbol] represents either a single, double, or triple bond, valency permitting. By way of example,

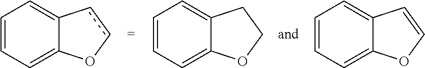

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other stereoisomers.

Compounds disclosed herein may exist as one or more tautomers. Tautomers are interconvertible structural isomers that differ in the position of one or more protons or other labile atom. By way of example:

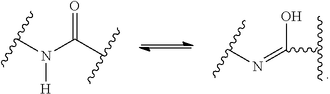

The prevalence of one tautomeric form over another will depend both on the specific chemical compound as well as its local chemical environment. Unless specified to the contrary, the depiction of one tautomeric form is inclusive of all possible tautomeric forms.

Unless stated to the contrary, a substituent drawn without explicitly specifying the point of attachment indicates that the substituent may be attached at any possible atom. For example, in a benzofuran depicted as:

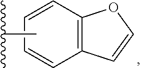

the substituent may be present at any one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

As used herein, “administration” refers to the injection of active agent on the subject. Exemplary methods of administration include: intravenously (i.v.), intraperitoneally (i.p.), intratumorally (i.t.), or subcutaneously (s.c.) such as tissue ipsilateral (i.l.) to the tumor and tissue contralateral (c.l.) to the tumor.

Disclosed herein are compounds having the formula:

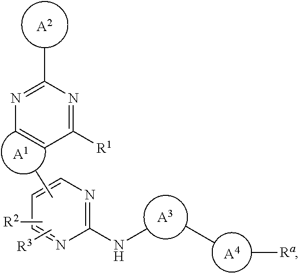

and pharmaceutically acceptable salts thereof, wherein
A¹ is aryl, cycloalkyl, heteroaryl, or heterocyclyl group
A² is an alkyl, cycloalkyl, heteroaryl or heterocyclyl group, or Q¹-NR^{6a}R^{6b}, wherein Q¹ is null or C₁₋₄alkylene, R^{6a} is hydrogen, C₁₋₈alkyl, aryl, C₁₋₈heteroaryl, C₃₋₈cycloalkyl, or C₁₋₈heterocyclyl, R^{6b} is hydrogen, C₁₋₈alkyl, aryl, C₁₋₈heteroaryl, C₃₋₈cycloalkyl, or C₁₋₈heterocyclyl;
A³ is null or an aryl, cycloalkyl, heteroaryl, or heterocyclyl group
A⁴ is null or an aryl, cycloalkyl, heteroaryl, or heterocyclyl group
R¹ is hydrogen, C₁₋₈alkyl, or C₃₋₈cycloalkyl;
R² is F, Cl, Br, I, NO₂, CN, R^{2*}, OR^{2*}, N(R^{2*})₂, SO₃R^{2*}, SO₂R^{2*}, SO₂N(R^{2*})₂, C(O)R^{2*}; C(O)OR^{2*}, OC(O)R^{2*}; C(O)N(R^{2*})₂, N(R^{2*})C(O)R^{2*}, OC(O)N(R^{2*})₂, N(R^{2*})C(O)N(R^{2*})₂, wherein R^{2*} is in each case independently selected from hydrogen, C₁₋₈alkyl, aryl, C₁₋₈heteroaryl, C₃₋₈cycloalkyl, or C₁₋₈heterocyclyl;
R³ is F, Cl, Br, I, NO₂, CN, R^{3*}, OR^{3*}, N(R^{3*})₂, SO₃R^{3*}, SO₂R^{3*}, SO₂N(R^{3*})₂, C(O)R^{3*}; C(O)OR^{3*}, OC(O)R^{3*}; C(O)N(R^{3*})₂, N(R^{3*})C(O)R^{3*}, OC(O)N(R^{3*})₂, N(R^{3*})C(O)N(R^{3*})₂, wherein R^{3*} is in each case independently selected from hydrogen, C₁₋₈alkyl, aryl, C₁₋₈heteroaryl, C₃₋₈cycloalkyl, or C₁₋₈heterocyclyl;
R^{a} has the formula:

—Z³—Z^{c}—Z⁴—Z^{t},
wherein
Z³ is null, C₁₋₆alkylene, C₃₋₈cycloalkylene, arylene, C₁₋₈hetercyclylene, or C₁₋₈heteroarylene,
Z^{c} is null, C(═O)—, SO₂, —C(═O)O—, —C(═O)NH—
Z⁴ is null, C₁₋₆alkylene, C₃₋₈cycloalkylene, arylene, C₁₋₈hetercyclylene, or C₁₋₈heteroarylene,
Z^{t} represents a bond to A⁴ or A³, or is F, Cl, Br, I, CN, NO₂, R^{za*}, OR^{za*}, SR^{za*}, N(R^{za*})₂, C(═O)R^{za*}, C(═O)OR^{za}), or C(═O)NHR^{za*},
wherein R^{za*} is in each case independently selected from hydrogen, C₁₋₈alkyl, aryl, C₁₋₈heteroaryl, C₃₋₈cycloalkyl, or C₁₋₈heterocyclyl;
wherein any two of more A¹, A², A³, A⁴, R¹, R², R³, and R^{a} may together form a ring.

In certain implementations, R¹ is hydrogen, methyl, ethyl, isopropyl, n-butyl, isobutyl, or cyclopropyl.

In some embodiments R² is F, Cl, Br, I, CN, R^{2*}, OR^{2*}, N(R^{2*})₂, wherein R^{2*} is in each case independently selected from hydrogen and C₁₋₈alkyl.

In some embodiments R² is H, F, CH₃, OCH₃, or CF₃.

In some embodiments R³ is F, Cl, Br, I, R^{3*}, OR^{3*}, or N(R^{3*})₂, wherein R^{3*} is in each case independently selected from hydrogen and C₁₋₈alkyl.

In some embodiments R³ is H, F, CH₃, OCH₃, or CF₃, preferably H.

In some embodiments, the compound has the formula:

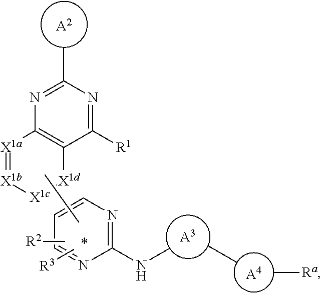

wherein
X^{1a} is selected from N and CR;
X^{2a} is selected from N and CR;
X^{3a} is selected from N and CR;
X^{4a} is selected from N and CR;
wherein one R group is a single bond to the *-pyrimidine, and the remaining R groups are independently selected from F, Cl, Br, I, NO₂, CN, R*, OR*, N(R*)₂, SO₃R*, SO₂R*, SO₂N(R*)₂, C(O)R*; C(O)OR*, OC(O)R*; C(O)N(R*)₂, N(R*)C(O)R*, OC(O)N(R*)₂, N(R*)C(O)N(R*)₂, wherein R* is in each case independently selected from hydrogen, C₁₋₈alkyl, aryl, C₁₋₈heteroaryl, C₃₋₈cycloalkyl, or C₁₋₈heterocyclyl.
wherein any two of more A¹, A², A³, A⁴, R¹, R², R³, R, and R^{a} may together form a ring.

In further embodiments, the compound has the formula:

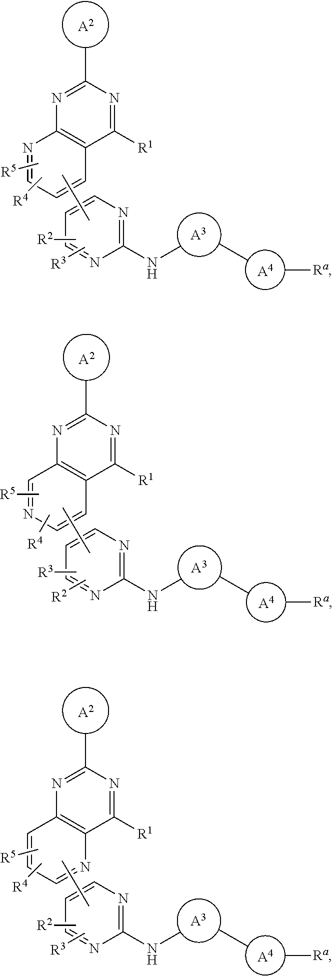

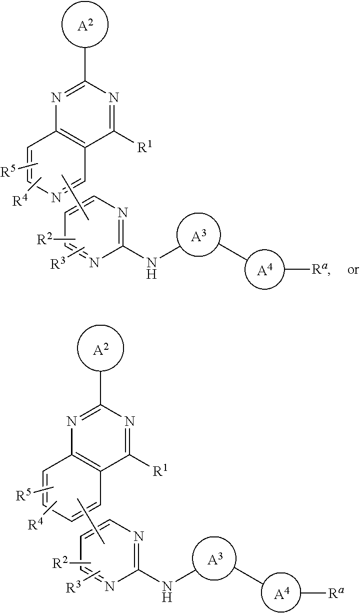

wherein
R⁴ is F, Cl, Br, I, NO₂, CN, R^{4*}, OR^{4*}, N(R^{4*})₂, SO₃R^{4*}, SO₂R^{4*}, SO₂N(R^{4*})₂, C(O)R^{4*}; C(O)OR^{4*}, OC(O)R^{4*}; C(O)N(R^{4*})₂, N(R^{4*})C(O)R^{4*}, OC(O)N(R^{4*})₂, N(R^{4*})C(O)N(R^{4*})₂, wherein R^{4*} is in each case independently selected from hydrogen, C₁₋₈alkyl, aryl, C₁₋₈heteroaryl, C₃₋₈cycloalkyl, or C₁₋₈heterocyclyl;
R⁵ is F, Cl, Br, I, NO₂, CN, R^{5*}, OR^{5*}, N(R^{5*})₂, SO₃R^{5*}, SO₂R^{5*}, SO₂N(R^{5*})₂, C(O)R^{5*}; C(O)OR^{5*}, OC(O)R^{5*}; C(O)N(R^{5*})₂, N(R^{5*})C(O)R^{5*}, OC(O)N(R^{5*})₂, N(R^{5*})C(O)N(R^{5*})₂, wherein R^{5*} is in each case independently selected from hydrogen, C₁₋₈alkyl, aryl, C₁₋₈heteroaryl, C₃₋₈cycloalkyl, or C₁₋₈heterocyclyl;
wherein any two of more of A², A³, A⁴, R¹, R², R³, R⁴, R⁵, and R^{a} may together form a ring.

In some embodiments R⁴ is F, Cl, Br, I, CN, R^{4*}, OR^{4*}, N(R^{4*})₂, wherein R^{4*} is in each case independently selected from hydrogen and C₁₋₈alkyl.

In some embodiments R⁴ is H, F, CH₃, OCH₃, or CF₃.

In some embodiments R⁵ is F, Cl, Br, I, CN, R^{5*}, OR^{5*}, N(R^{5*})₂, wherein R^{5*} is in each case independently selected from hydrogen and C₁₋₈alkyl.

In some embodiments R⁵ is H, F, CH₃, OCH₃, or CF₃.

In some embodiments the compound has the formula:

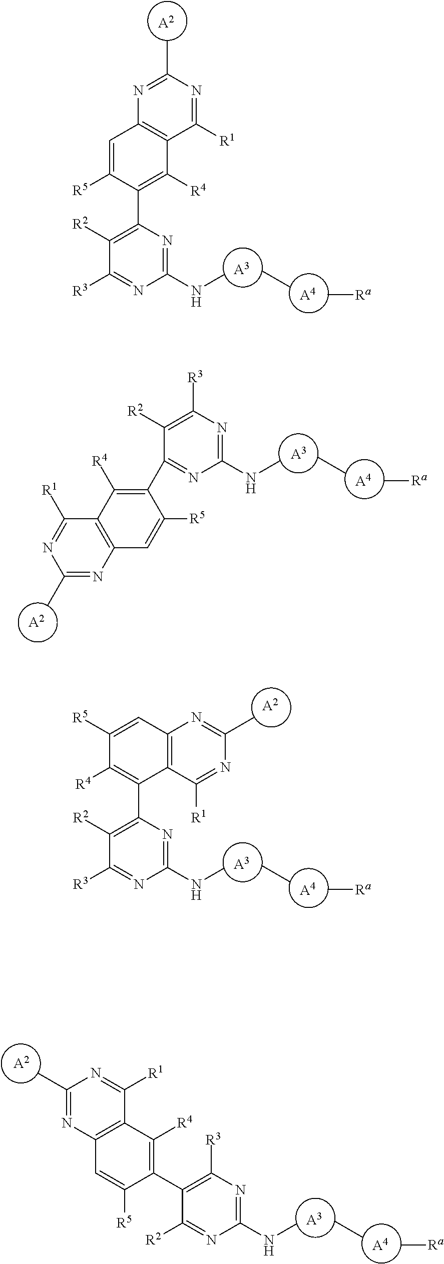

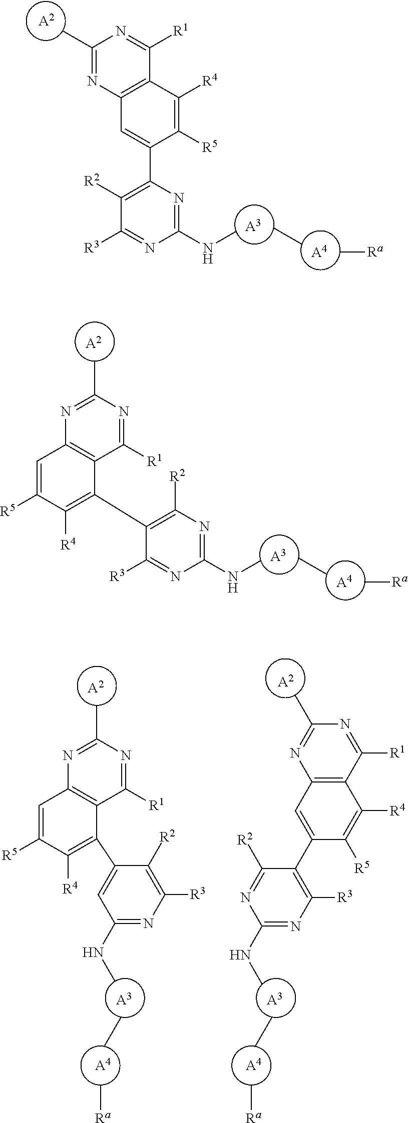

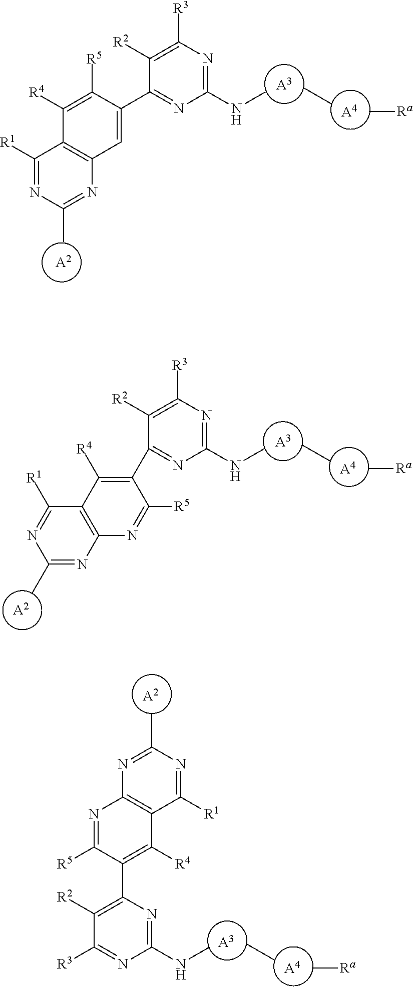

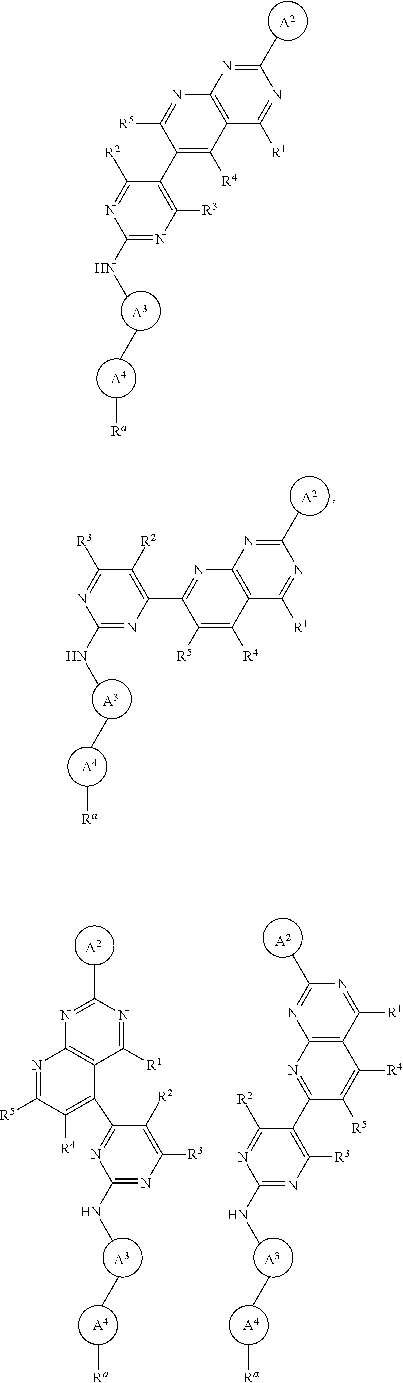

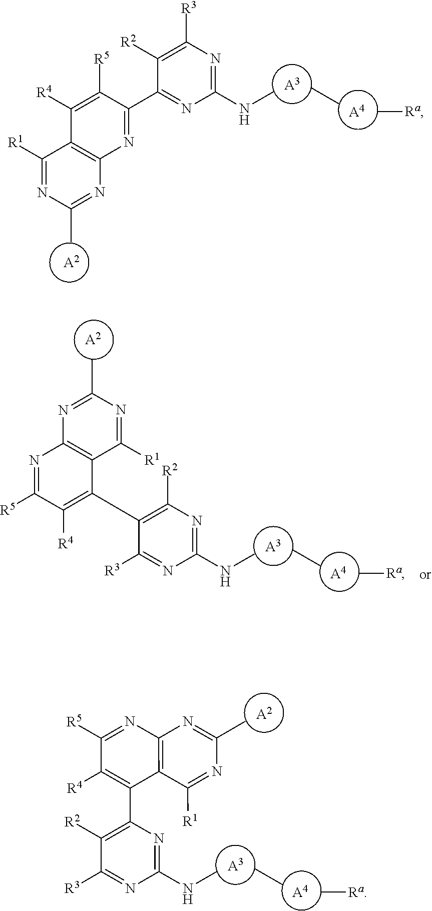

In certain implementations, the compound has the structure:

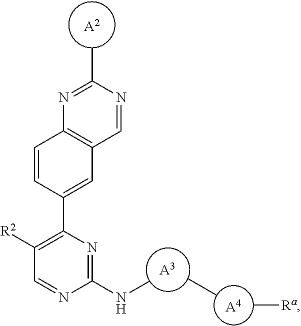

wherein R² is H or F.

In some embodiments A² has the formula:

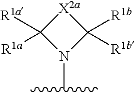

wherein X^{a2} is selected from Z, Z—O, Z—S, Z—NRⁿ¹,
wherein Z is null or a group having the formula:

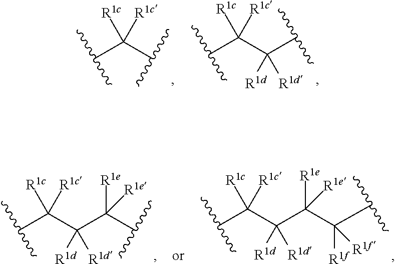

wherein Rⁿ¹ is selected from is hydrogen or C₁₋₈alkyl;
R^{1a} is selected from F, Cl, Br, I, NO₂, CN, R^{1a*}, OR^{1a*}, N(R^{1a*})₂, SO₃R^{1a*}, SO₂R^{1a*}, SO₂N(R^{1a*})₂, C(O)R^{1a*}; C(O)OR^{1a*}, OC(O)R^{1a*}; C(O)N(R^{1a*})₂, N(R^{1a*})C(O)R^{1a*}, OC(O)N(R^{1a*})₂, N(R^{1a*})C(O)N(R^{1a*})₂, wherein R^{1a*} is in each case independently selected from hydrogen, C₁₋₈alkyl, aryl, C₁₋₈heteroaryl, C₃₋₈cycloalkyl, or C₁₋₈heterocyclyl;
R^{1a′} is selected from F, Cl, Br, I, NO₂, CN, R^{1a′*}, OR^{1a′*}, N(R^{1a′*})₂, SO₃R^{1a′*}, SO₂R^{1a′*}, SO₂N(R^{1a′*})₂, C(O)R^{1a′*}; C(O)OR^{1a′*}, OC(O)R^{1a′*}; C(O)N(R^{1a′*})₂, N(R^{1a′*})C(O)R^{1a′*}, OC(O)N(R^{1a′*})₂, N(R^{1a′*})C(O)N(R^{1a′*})₂, wherein R^{1a′*} is in each case independently selected from hydrogen, C₁₋₈alkyl, aryl, C₁₋₈heteroaryl, C₃₋₈cycloalkyl, or C₁₋₈heterocyclyl; or
wherein R^{1a} and R^{1a′} together form an oxo, or thiooxo;
R^{1b} is selected from F, Cl, Br, I, NO₂, CN, R^{1b*}, OR^{1b*}, N(R^{1b*})₂, SO₃R^{1b*}, SO₂R^{1b*}, SO₂N(R^{1b*})₂, C(O)R^{1b*}; C(O)OR^{1b*}, OC(O)R^{1b*}; C(O)N(R^{1b*})₂, N(R^{1b*})C(O)R^{1b*}, OC(O)N(R^{1b*})₂, N(R^{1b*})C(O)N(R^{1b*})₂, wherein R^{1b*} is in each case independently selected from hydrogen, C₁₋₈alkyl, aryl, C₁₋₈heteroaryl, C₃₋₈cycloalkyl, or C₁₋₈heterocyclyl;
R^{1b′} is selected from F, Cl, Br, I, NO₂, CN, R^{1b′*}, OR^{1b′*}, N(R^{1b′*})₂, SO₃R^{1b′*}, SO₂R^{1b′*}, SO₂N(R^{1b′*})₂, C(O)R^{1b′*}; C(O)OR^{1b′*}, OC(O)R^{1b′*}; C(O)N(R^{1b′*})₂, N(R^{1b′*})C(O)R^{1b′*}, OC(O)N(R^{1b′*})₂, N(R^{1b′*})C(O)N(R^{1b′*})₂, wherein R^{1b′*} is in each case independently selected from hydrogen, C₁₋₈alkyl, aryl, C₁₋₈heteroaryl, C₃₋₈cycloalkyl, or C₁₋₈heterocyclyl; or
wherein R^{1b} and R^{1b′} together form an oxo, or thiooxo
R^{1c} is selected from F, Cl, Br, I, NO₂, CN, R^{1c*}, OR^{1c*}, N(R^{1c*})₂, SO₃R^{1c*}, SO₂R^{1c*}, SO₂N(R^{1c*})₂, C(O)R^{1c*}; C(O)OR^{1c*}, OC(O)R^{1c*}; C(O)N(R^{1c*})₂, N(R^{1c*})C(O)R^{1c*}, OC(O)N(R^{1c*})₂, N(R^{1c′*})C(O)N(R^{1c*})₂, wherein R^{1c*} is in each case independently selected from hydrogen, C₁₋₈alkyl, aryl, C₁₋₈heteroaryl, C₃₋₈cycloalkyl, or C₁₋₈heterocyclyl;
R^{1c′} is selected from F, Cl, Br, I, NO₂, CN, R^{1c′*}, OR^{1c′*}, N(R^{1c′*})₂, SO₃R^{1c′*}, SO₂R^{1c′*}, SO₂N(R^{1c′*})₂, C(O)R^{1c′*}; C(O)OR^{1c′*}, OC(O)R^{1c′*}; C(O)N(R^{1c′*})₂, N(R^{1c′*})C(O)R^{1c′*}, OC(O)N(R^{1c′*})₂, N(R^{1c′*})C(O)N(R^{1c*})₂, wherein R^{1c′*} is in each case independently selected from hydrogen, C₁₋₈alkyl, aryl, C₁₋₈heteroaryl, C₃₋₈cycloalkyl, or C₁₋₈heterocyclyl; or
wherein R^{1c} and R^{1c′} together form an oxo, or thiooxo
R^{1d} is selected from F, Cl, Br, I, NO₂, CN, R^{1d*}, OR^{1d*}, N(R^{1d*})₂, SO₃R^{1d*}, SO₂R^{1d*}, SO₂N(R^{1d*})₂, C(O)R^{1d*}; C(O)OR^{1d*}, OC(O)R^{1d*}; C(O)N(R^{1d*})₂, N(R^{1d*})C(O)R^{1d*}, OC(O)N(R^{1d*})₂, N(R^{1d*})C(O)N(R^{1d*})₂, wherein R^{1d*} is in each case independently selected from hydrogen, C₁₋₈alkyl, aryl, C₁₋₈heteroaryl, C₃₋₈cycloalkyl, or C₁₋₈heterocyclyl;
R^{1d′} is selected from F, Cl, Br, I, NO₂, CN, R^{1d′*}, OR^{1d′*}, N(R^{1d′*})₂, SO₃R^{1d′*}, SO₂R^{1d′*}, SO₂N(R^{1d′*})₂, C(O)R^{1d′*}; C(O)OR^{1d′*}, OC(O)R^{1d′*}; C(O)N(R^{1d′*})₂, N(R^{1d′*})C(O)R^{1d′*}, OC(O)N(R^{1d′*})₂, N(R^{1d′*})C(O)N(R^{1d′*})₂, wherein R^{1d′*} is in each case independently selected from hydrogen, C₁₋₈alkyl, aryl, C₁₋₈heteroaryl, C₃₋₈cycloalkyl, or C₁₋₈heterocyclyl; or
wherein R^{1d} and R^{1d′} together form an oxo, or thiooxo
R^{1e} is selected from F, Cl, Br, I, NO₂, CN, R^{1e*}, OR^{1e*}, N(R^{1e*})₂, SO₃R^{1e*}, SO₂R^{1e*}, SO₂N(R^{1e*})₂, C(O)R^{1e*}; C(O)OR^{1e*}, OC(O)R^{1e*}; C(O)N(R^{1e*})₂, N(R^{1e*})C(O)R^{1e*}, OC(O)N(R^{1e*})₂, N(R^{1e*})C(O)N(R^{1e*})₂, wherein R^{1e*} is in each case independently selected from hydrogen, C₁₋₈alkyl, aryl, C₁₋₈heteroaryl, C₃₋₈cycloalkyl, or C₁₋₈heterocyclyl;
R^{1e′} is selected from F, Cl, Br, I, NO₂, CN, R^{1e′*}, OR^{1e′*}, N(R^{1e′*})₂, SO₃R^{1e′*}, SO₂R^{1e′*}, SO₂N(R^{1e′*})₂, C(O)R^{1e′*}; C(O)OR^{1e′*}, OC(O)R^{1e′*}; C(O)N(R^{1e′*})₂, N(R^{1e′*})C(O)R^{1e′*}, OC(O)N(R^{1e′*})₂, N(R^{1e′*})C(O)N(R^{1e′*})₂, wherein R^{1e′*} is in each case independently selected from hydrogen, C₁₋₈alkyl, aryl, C₁₋₈heteroaryl, C₃₋₈cycloalkyl, or C₁₋₈heterocyclyl; or
wherein R^{1e} and R^{1e′} together form an oxo, or thiooxo
R^{1f} is selected from F, Cl, Br, I, NO₂, CN, R^{1f*}, OR^{1f*}, N(R^{1f*})₂, SO₃R^{1f*}, SO₂R^{1f*}, SO₂N(R^{1f*})₂, C(O)R^{1f*}; C(O)OR^{1f*}, OC(O)R^{1f*}; C(O)N(R^{1f*})₂, N(R^{1f*})C(O)R^{1f*}, OC(O)N(R^{1f*})₂, N(R^{1f*})C(O)N(R^{1f*})₂, wherein R^{1f*} is in each case independently selected from hydrogen, C₁₋₈alkyl, aryl, C₁₋₈heteroaryl, C₃₋₈cycloalkyl, or C₁₋₈heterocyclyl;
R^{1f′} is selected from F, Cl, Br, I, NO₂, CN, R^{1f′*}, OR^{1f′*}, N(R^{1f′*})₂, SO₃R^{1f′*}, SO₂R^{1f′*}, SO₂N(R^{1f′*})₂, C(O)R^{1f′*}; C(O)OR^{1f′*}, OC(O)R^{1f′*}; C(O)N(R^{1f′*})₂, N(R^{1f′*})C(O)R^{1f′*}, OC(O)N(R^{1f′*})₂, N(R^{1f′*})C(O)N(R^{1f′*})₂, wherein R^{1f′*} is in each case independently selected from hydrogen, C₁₋₈alkyl, aryl, C₁₋₈heteroaryl, C₃₋₈cycloalkyl, or C₁₋₈heterocyclyl; or
wherein R^{1f} and R^{1f′} together form an oxo, or thiooxo;
wherein any two adjacent R groups may together form a double bond;
wherein any two or more of R^{1a}, R^{1a′}, R^{1b}, R^{1c}, R^{1d}, R^{1d′}, R^{1d}, R^{1d′}, R^{1e}, R^{1e′}, R^{1f}, R^{1f′}, and Rⁿ¹ may together form a ring.

In some embodiments R^{1a} and R^{1a′} form an oxo, or wherein R^{1a} is F, Cl, Br, I, CN, R^{1a*}, OR^{1a*}, N(R^{1a*})₂, wherein R^{1a*} is in each case independently selected from hydrogen and C₁₋₈alkyl, and R^{1a′} together with another R group forms a ring, or is H.

In some embodiments R^{1b} and R^{1b′} form an oxo, or wherein R^{1b} is F, Cl, Br, I, CN, R^{1b*}, OR^{1b*}, N(R^{1b*})₂, wherein R^{1b*} is in each case independently selected from hydrogen and C₁₋₈alkyl, and R^{1b′} together with another R group forms a ring, or is H.

In some embodiments R^{1c} and R^{1c′} form an oxo, or wherein R^{1c} is F, Cl, Br, I, CN, R^{1c*}, OR^{1c*}, N(R^{1c*})₂, wherein R^{1c*} is in each case independently selected from hydrogen and C₁₋₈alkyl, and R^{1c′} together with another R group forms a ring, or is H.

In some embodiments R^{1b} and R^{1d′} form an oxo, or wherein R^{1d} is F, Cl, Br, I, CN, R^{1d*}, OR^{1d*}, N(R^{1d*})₂, wherein R^{1d*} is in each case independently selected from hydrogen and C₁₋₈alkyl, and R^{1d′} together with another R group forms a ring, or is H.

In some embodiments R^{1e} and R^{1e′} form an oxo, or wherein R^{1e} is F, Cl, Br, I, CN, R^{1e*}, OR^{1e*}, N(R^{1e*})₂, wherein R^{1e′*} is in each case independently selected from hydrogen and C₁₋₈alkyl, and R^{1e′} together with another R group forms a ring, or is H.

In some embodiments R^{1f} and R^{1f′} form an oxo, or wherein R^{1f} is F, Cl, Br, I, CN, R^{1f*}, OR^{1f*}, N(R^{1f*})₂, wherein R^{1f*} is in each case independently selected from hydrogen and C₁₋₈alkyl, and R^{1f} together with another R group forms a ring, or is H.

In certain implementations A² has the formula:

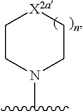

wherein X^{2a′} is CH₂, O, or NRⁿ¹, and n is selected from 0, 1, or 2.

In some embodiments A² has the formula:

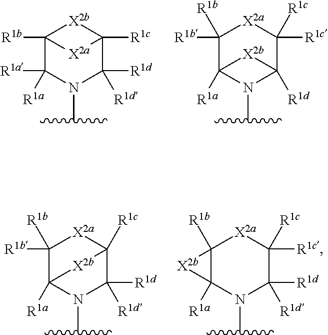

wherein X^{2b} is selected from Z¹, Z¹—O—Z¹, Z—S—Z¹, Z¹—NR²—Z¹, wherein Z¹ is null or a group having the formula:

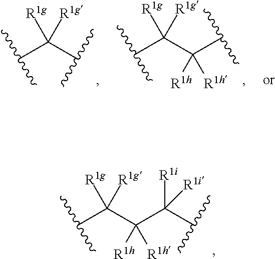

wherein Rⁿ² is selected from H or C₁₋₈alkyl,
wherein R^{1g} is selected from F, Cl, Br, I, NO₂, CN, R^{1g*}, OR^{1g*}, N(R^{1g*})₂, SO₃R^{1g*}, SO₂R^{1g*}, SO₂N(R^{1g*})₂, C(O)R^{1g*}; C(O)OR^{1g*}, OC(O)R^{1g*}; C(O)N(R^{1g*})₂, N(R^{1g*})C(O)R^{1g*}, OC(O)N(R^{1g*})₂, N(R^{1g*})C(O)N(R^{1g*})₂, wherein R^{1g*} is in each case independently selected from hydrogen, C₁₋₈alkyl, aryl, C₁₋₈heteroaryl, C₃₋₈cycloalkyl, or C₁₋₈heterocyclyl;
wherein R^{1g′} is selected from F, Cl, Br, I, NO₂, CN, R^{1g′*}, OR^{1g′*}, N(R^{1g′*})₂, SO₃R^{1g′*}, SO₂R^{1g′*}, SO₂N(R^{1g′*})₂, C(O)R^{1g′*}; C(O)OR^{1g′*}, OC(O)R^{1g′*}; C(O)N(R^{1g′*})₂, N(R^{1g′*})C(O)R^{1g′*}, OC(O)N(R^{1g′*})₂, N(R^{1g′*})C(O)N(R^{1g′*})₂, wherein R^{1g′*} is in each case independently selected from hydrogen, C₁₋₈alkyl, aryl, C₁₋₈heteroaryl, C₃₋₈cycloalkyl, or C₁₋₈heterocyclyl; or
wherein R^{1g} and R^{1g′} together form an oxo, or thiooxo
wherein R^{1h} is selected from F, Cl, Br, I, NO₂, CN, R^{1h*}, OR^{1h*}, N(R^{1h*})₂, SO₃R^{1h*} SO₂R^{1h*}, SO₂N(R^{1h*})₂, C(O)R^{1h*}; C(O)OR^{1h*}, OC(O)R^{1h*}; C(O)N(R^{1h*})₂, N(R^{1h*})C(O)R^{1h*}, OC(O)N(R^{1h*})₂, N(R^{1h*})C(O)N(R^{1h*})₂, wherein R^{1h*} is in each case independently selected from hydrogen, C₁₋₈alkyl, aryl, C₁₋₈heteroaryl, C₃₋₈cycloalkyl, or C₁₋₈heterocyclyl;
wherein R^{1h′} is selected from F, Cl, Br, I, NO₂, CN, R^{1h′*}, OR^{1h′*}, N(R^{1h′*})₂, SO₃R^{1h′*} SO₂R^{1h′*}, SO₂N(R^{1h′*})₂, C(O)R^{1h′*}; C(O)OR^{1h′*}, OC(O)R^{1h′*}; C(O)N(R^{1h′*})₂, N(R^{1h′*})C(O)R^{1h′*}, OC(O)N(R^{1h′*})₂, N(R^{1h′*})C(O)N(R^{1h′*})₂, wherein R^{1h′*} is in each case independently selected from hydrogen, C₁₋₈alkyl, aryl, C₁₋₈heteroaryl, C₃₋₈cycloalkyl, or C₁₋₈heterocyclyl; or
wherein R^{1h} and R^{1h′} together form an oxo, or thiooxo
wherein R^{h} is selected from F, Cl, Br, I, NO₂, CN, R^{1i*}, OR^{1i*}, N(R^{1i*})₂, SO₃R^{1i*}, SO₂R^{1i*}, SO₂N(R^{1i*})₂, C(O)R^{1i*}; C(O)OR^{1i*}, OC(O)R^{1i*}; C(O)N(R^{1i*})₂, N(R^{1i*})C(O)R^{1i*}, OC(O)N(R^{1i*})₂, N(R^{1i*})C(O)N(R^{1i*})₂, wherein R^{1i*} is in each case independently selected from hydrogen, C₁₋₈alkyl, aryl, C₁₋₈heteroaryl, C₃₋₈cycloalkyl, or C₁₋₈heterocyclyl;
wherein R^{1i′} is selected from F, Cl, Br, I, NO₂, CN, R^{1i′*}, OR^{1i′*}, N(R^{1i′*})₂, SO₃R^{1i′*}, SO₂R^{1i′*}, SO₂N(R^{1i′*})₂, C(O)R^{1i′*}; C(O)OR^{1i′*}, OC(O)R^{1i′*}; C(O)N(R^{1i′*})₂, N(R^{1i′*})C(O)R^{1i′*}, OC(O)N(R^{1i′*})₂, N(R^{1i′*})C(O)N(R^{1i′*})₂, wherein R^{1i′*} is in each case independently selected from hydrogen, C₁₋₈alkyl, aryl, C₁₋₈heteroaryl, C₃₋₈cycloalkyl, or C₁₋₈heterocyclyl; or
wherein R¹ⁱ and R^{1i′ together form an oxo, or thiooxo}
wherein any two adjacent R groups may together form a double bond;
wherein any two or more of R^{1a}, R^{1a′}, R^{1b}, R^{1b′}, R^{1c}, R^{1c′}, R^{1d}, R^{1d′}, R^{1e}, R^{1e′}, R^{1f}, R^{1f′}, R^{1g}, R^{1g′}, R^{1g}, R^{1g′}, R¹ⁱ, and R^{1i′} may together form a ring;

In some embodiments X^{2b} is selected from null, CH₂, CH₂CH₂, CH₂CH₂CH₂, OC(═O), SC(═O), NHC(═O), N(CH₃)C(═O), CH₂C(═O), OCH₂, SCH₂, NHCH₂, N(CH₃)CH₂, CH₂C(═O)CH₂, CH₂OCH₂, CH₂SCH₂, CH₂NHCH₂, CH₂N(CH₃)CH₂, CH₂C(═O)CH₂, CH₂C(═O)O, CH₂C(═O)S, CH₂C(═O)NH, or CH₂C(═O)N(CH₃), In some embodiments R^{1g} and R^{1g′} form an oxo, or wherein R^{1g} is F, Cl, Br, I, CN, R^{1g*}, OR^{1g*}, N(R^{1g*})₂, wherein R^{1g*} is in each case independently selected from hydrogen and C₁₋₈alkyl, and R^{1g′} is H.

In some embodiments R^{1h} and R^{1h′} form an oxo, or wherein R^{1h} is F, Cl, Br, I, CN, R^{1h*}, OR^{1h*}, N(R^{1h*})₂, wherein R^{1h*} is in each case independently selected from hydrogen and C₁₋₈alkyl, and R^{1h′} is H.

In some embodiments R¹ⁱ and R^{1i′} form an oxo, or wherein R¹ⁱ is F, Cl, Br, I, CN, R^{1i*}, OR^{1i*}, N(R^{1i*})₂, wherein R^{1i*} is in each case independently selected from hydrogen and C₁₋₈alkyl, and R^{1i′} is H.

In some embodiments A² has the formula:

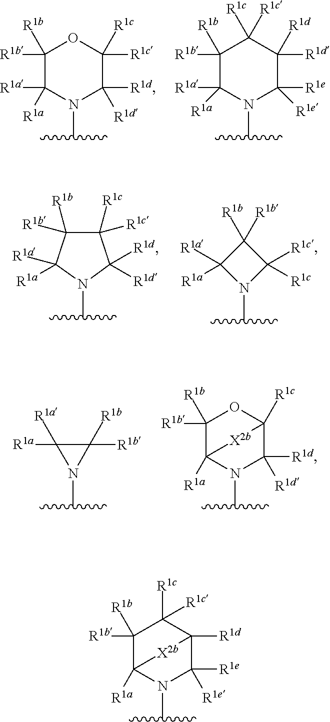

wherein X^{2b} is:

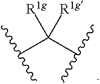

In some embodiments R^{1a}, R^{2b}, R^{1c}, R^{1d}, and R^{1e} are each hydrogen.

In some embodiments R^{1a′}, R^{2b′}, R^{1c′}, R^{1d′}, and R^{1e′} are each hydrogen.

In some embodiments A² has the formula:

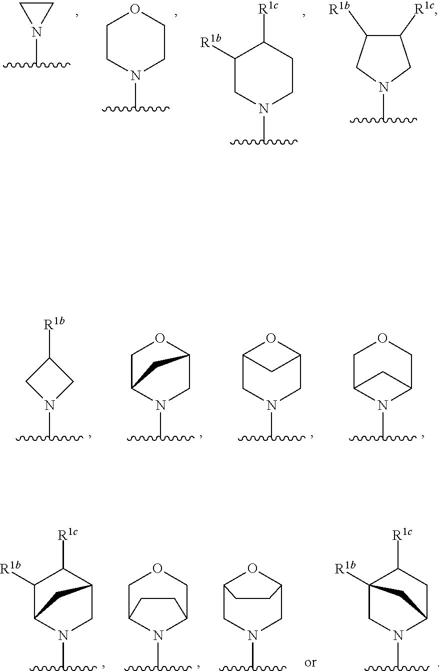

In some embodiments A² has the formula:

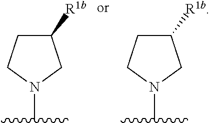

In some embodiments R^{1a} and R^{1a′} are both H.

In some embodiments R^{1b} is H and R^{1b′} is H or F.

In some embodiments R^{1c} is H and R^{1c′} is H or F.

In some embodiments R^{1d} and R^{1d′} are both H.

In some embodiments R^{1e} and R^{1e}′ are both H.

In some embodiments A² is NH₂, NHCH₃, N(CH₃)₂, NHCH₂CH₂OH, N(CH₂CH₂OH)₂, CH₂NH₂, CH₂NHCH₃, CH₂N(CH₃)₂, CH₂NHCH₂CH₂OH, CH₂N(CH₂CH₂OH)₂, CH₂CH₂NH₂, CH₂CH₂NHCH₃, CH₂CH₂N(CH₃)₂, CH₂CH₂NHCH₂CH₂OH, or CH₂CH₂N(CH₂CH₂OH)₂.

In some embodiments A³ has the formula:

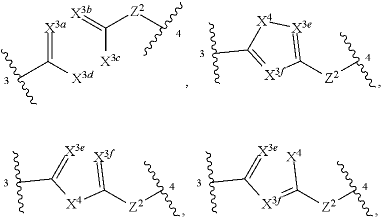

wherein
X^{3a} is CR^{3a} or N,
X^{3b} is CR^{3b} or N,
X^{3c} is CR^{3c} or N,
X^{3d} is CR^{3d} or N,
X^{3e} is CR^{3e} or N,
X^{3f} is CR^{3f} or N,
X⁴ is NRⁿ³, S, O,
Z² is null, O, S, NRⁿ⁴;
R^{3a} is F, Cl, Br, I, NO₂, CN, R^{3a*}, OR^{3a*}, N(R^{3a*})₂, SO₃R^{3a*}, SO₂R^{3a*}, SO₂N(R^{3a*})₂, C(O)R^{3a*}; C(O)OR^{3a*}, OC(O)R^{3a*}; C(O)N(R^{3a*})₂, N(R^{3a*})C(O)R^{3a*}, OC(O)N(R^{3a*})₂, N(R^{3a*})C(O)N(R^{3a*})₂, wherein R^{3a*} is in each case independently selected from hydrogen, C₁₋₈alkyl, aryl, C₁₋₈heteroaryl, C₃₋₈cycloalkyl, or C₁₋₈heterocyclyl;
R^{3b} is F, Cl, Br, I, NO₂, CN, R^{3b*}, OR^{3b*}, N(R^{3b*})₂, SO₃R^{3b*}, SO₂R^{3b*}, SO₂N(R^{3b*})₂, C(O)R^{3b*}; C(O)OR^{3b*}, OC(O)R^{3b*}; C(O)N(R^{3b*})₂, N(R^{3b*})C(O)R^{3b*}, OC(O)N(R^{3b*})₂, N(R^{3b*})C(O)N(R^{3b*})₂, wherein R^{3b*} is in each case independently selected from hydrogen, C₁₋₈alkyl, aryl, C₁₋₈heteroaryl, C₃₋₈cycloalkyl, or C₁₋₈heterocyclyl;
R^{3c} is F, Cl, Br, I, NO₂, CN, R^{3c*}, OR^{3c*}, N(R^{3c*})₂, SO₃R^{3c*}, SO₂R^{3c*}, SO₂N(R^{3c*})₂, C(O)R^{3c*}; C(O)OR^{3c*}, OC(O)R^{3c*}; C(O)N(R^{3c*})₂, N(R^{3c*})C(O)R^{3c*}, OC(O)N(R^{3c*})₂, N(R^{3c*})C(O)N(R^{3c*})₂, wherein R^{3c*} is in each case independently selected from hydrogen, C₁₋₈alkyl, aryl, C₁₋₈heteroaryl, C₃₋₈cycloalkyl, or C₁₋₈heterocyclyl;
R^{3d} is F, Cl, Br, I, NO₂, CN, R^{3d*}, OR^{3d*}, N(R^{3d*})₂, SO₃R^{3d*}, SO₂R^{3d*}, SO₂N(R^{3d*})₂, C(O)R^{3d*}; C(O)OR^{3d*}, OC(O)R^{3d*}; C(O)N(R^{3d*})₂, N(R^{3d*})C(O)R^{3d*}, OC(O)N(R^{3d*})₂, N(R^{3d*})C(O)N(R^{3d*})₂, wherein R^{3d*} is in each case independently selected from hydrogen, C₁₋₈alkyl, aryl, C₁₋₈heteroaryl, C₃₋₈cycloalkyl, or C₁₋₈heterocyclyl;
R^{3e} is F, Cl, Br, I, NO₂, CN, R^{3e*}, OR^{3e*}, N(R^{3e*})₂, SO₃R^{3e*}, SO₂R^{3e*}, SO₂N(R^{3e*})₂, C(O)R^{3e*}; C(O)OR^{3e*}, OC(O)R^{3e*}; C(O)N(R^{3e*})₂, N(R^{3e*})C(O)R^{3e*}, OC(O)N(R^{3e*})₂, N(R^{3e*})C(O)N(R^{3e*})₂, wherein R^{3e*} is in each case independently selected from hydrogen, C₁₋₈alkyl, aryl, C₁₋₈heteroaryl, C₃₋₈cycloalkyl, or C₁₋₈heterocyclyl;
R^{3f} is F, Cl, Br, I, NO₂, CN, R^{3f*}, OR^{3f*}, N(R^{3f*})₂, SO₃R^{3f*}, SO₂R^{3f*}, SO₂N(R^{3f*})₂, C(O)R^{3f*}; C(O)OR^{3f*}, OC(O)R^{3f*}; C(O)N(R^{3f*})₂, N(R^{3f*})C(O)R^{3f*}, OC(O)N(R^{3f*})₂, N(R^{3f*})C(O)N(R^{3f*})₂, wherein R^{3f*} is in each case independently selected from hydrogen, C₁₋₈alkyl, aryl, C₁₋₈heteroaryl, C₃₋₈cycloalkyl, or C₁₋₈heterocyclyl;
R^{g} is F, Cl, Br, I, NO₂, CN, R^{3g*}, OR^{3g*}, N(R^{3g*})₂, SO₃R^{3g*}, SO₂R^{3g*}, SO_{2N}(R^{3g*})₂, C(O)R^{3g*}; C(O)OR^{3g*}, OC(O)R^{3g*}; C(O)N(R^{3g*})₂, N(R^{3g*})C(O)R^{3f*}, OC(O)N(R^{3g*})₂, N(R^{3g*})C(O)N(R^{3g*})₂, wherein R^{3g*} is in each case independently selected from hydrogen, C₁₋₈alkyl, aryl, C₁₋₈heteroaryl, C₃₋₈cycloalkyl, or C₁₋₈heterocyclyl;
Rⁿ³ is hydrogen, C₁₋₈alkyl, aryl, C₁₋₈heteroaryl, C₃₋₈cycloalkyl, or C₁₋₈heterocyclyl;
Rⁿ⁴ is hydrogen, C₁₋₈alkyl, aryl, C₁₋₈heteroaryl, C₃₋₈cycloalkyl, or C₁₋₈heterocyclyl;
wherein any one of R^{3a}, R^{3b}, R^{3c} R^{3d}, R^{3e}, R^{3f}, Rⁿ³, or Rⁿ⁴ may form a bond with Z^{t},
R^{3a} and R^{3b} may together form a ring;
R^{3c} and R^{3d} may together form a ring;
any two of R^{3e}, R^{3f}, and Rⁿ³ may together form a ring;
wavy line 3 represents the point of attachment to the aminopyrimidine group, and
wavy line 4 represents the point of attachment to A⁴;
wherein any two or more of R^{1a}, R^{1a′}, R^{1b}, R^{1b′}, R^{1c}, R^{1c′}, R^{1d}, R^{1d′}, R^{1e}, R^{1e′}, R^{1f}, R^{1f′}, R^{1f′}, R^{1g}, R^{1g′}, R^{1g}, R^{1g′}, R¹ⁱ, R¹ⁱ, R^{3a}, R^{3b}, R^{3c}, R^{3a}, R^{3e}, R^{3f}, R^{3g}, Rⁿ², and Rⁿ³, and A⁴ may together form a ring;

In some embodiments A³ has the formula:

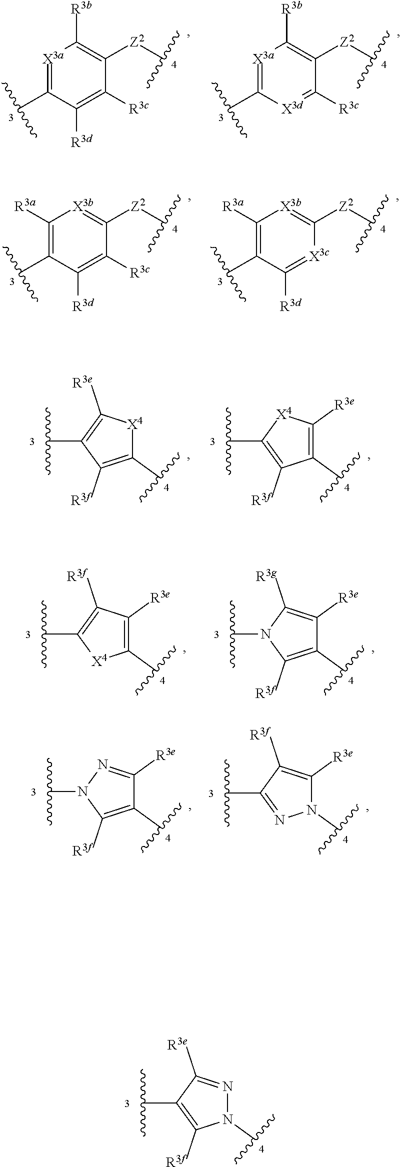

In some embodiments A³ has the formula:

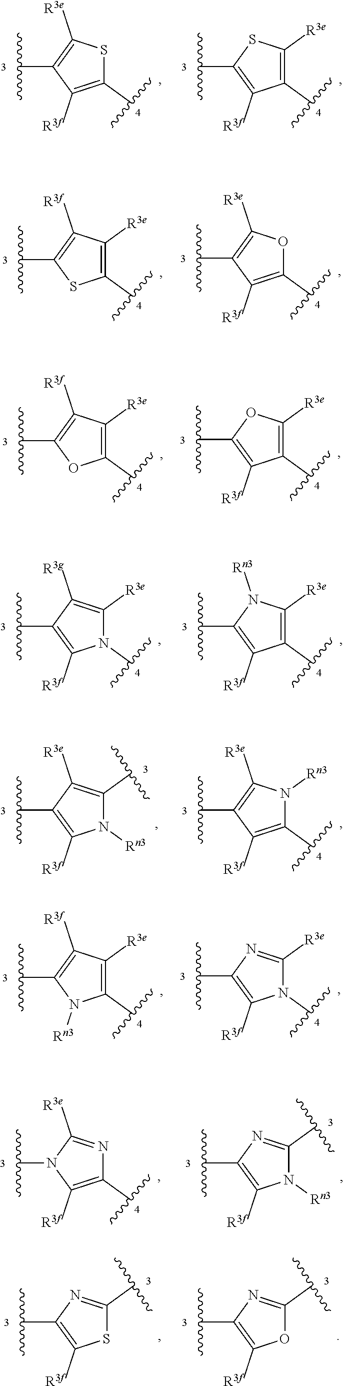

In some embodiments R^{3e}, R^{3f} and R^{3g} are each hydrogen. In some embodiments R¹³ is hydrogen or methyl.

In some embodiments R^{3e} is F, Cl, Br, CN, OC₁₋₃alkyl, OC₁₋₃haloalkyl, or C₁₋₃alkyl, and R^{3f} and R^{3g} are both hydrogen.

In some embodiments R^{3f} is F, Cl, Br, CN, OC₁₋₃alkyl, OC₁₋₃haloalkyl, or C₁₋₃alkyl, and R^{3f} and R^{3g} are both hydrogen.

In some embodiments R^{3g} is F, Cl, Br, CN, OC₁₋₃alkyl, OC₁₋₃haloalkyl, or C₁₋₃alkyl, and R^{3e} and R^{3f} are both hydrogen.

In some embodiments A³ has the formula:

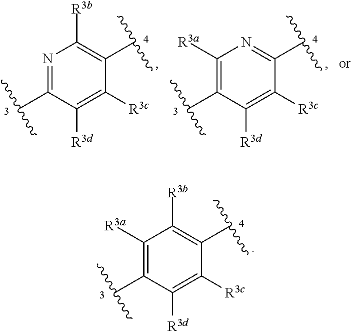

In some embodiments
R^{3a} is F, Cl, Br, I, CN, R^{3a*}, OR^{3a*}, N(R^{3a*})₂, C(O)R^{3a*}; C(O)OR^{3a*}, C(O)N(R^{3a*})₂, N(R^{3a*})C(O)R^{3a*}, wherein R^{3a*} is in each case independently selected from hydrogen, C₁₋₄alkyl, aryl, C₁₋₆heteroaryl, C₃₋₈cycloalkyl, or C₁₋₆heterocyclyl;
R^{3b} is F, Cl, Br, I, CN, R^{3b*}, OR^{3b*}, N(R^{3b*})₂, C(O)R^{3b*}; C(O)OR^{3b*}, C(O)N(R^{3b*})₂, N(R^{3b*})C(O)R^{3b*}, wherein R^{3b*} is in each case independently selected from hydrogen, C₁₋₄alkyl, aryl, C₁₋₆heteroaryl, C₃₋₈cycloalkyl, or C₁₋₆heterocyclyl;
R^{3c} is F, Cl, Br, I, CN, R^{3c*}, OR^{3c*}, N(R^{3c*})₂, C(O)R^{3c*}; C(O)OR^{3c*}; C(O)N(R^{3c*})₂, N(R^{3c*})C(O)R^{3c*}, wherein R^{3c*} is in each case independently selected from hydrogen, C₁₋₄alkyl, aryl, C₁₋₆heteroaryl, C₃₋₈cycloalkyl, or C₁₋₆heterocyclyl;
R^{3d} is F, Cl, Br, I, CN, R^{3d*}, OR^{3d*}, N(R^{3d*})₂, C(O)R^{3d*}; C(O)OR^{3d*}, C(O)N(R^{3d*})₂, N(R^{3d*})C(O)R^{3d*}, OC(O)N(R^{3d*})₂, wherein R^{3d*} is in each case independently selected from hydrogen, C₁₋₄alkyl, aryl, C₁₋₆heteroaryl, C₃₋₈cycloalkyl, or C₁₋₆heterocyclyl.

In some embodiments R^{3a} is F, Cl, Br, I, CN, R^{3a*}, OR^{3a*}, or N(R^{3a*})₂, wherein R^{3a*} is in each case independently selected from hydrogen or C₁₋₄alkyl;
R^{3b} is F, Cl, Br, I, CN, R^{3b*}, OR^{3b*}, or N(R^{3b*})₂, wherein R^{3b*} is in each case independently selected from hydrogen or C₁₋₄alkyl;
R^{3c} is F, Cl, Br, I, CN, R^{3c*}, OR^{3c*}, or N(R^{3c*})₂, wherein R^{3c*} is in each case independently selected from hydrogen or C₁₋₄alkyl;
R^{3d} is F, Cl, Br, I, CN, R^{3d*}, OR^{3d*}, or N(R^{3d*})₂, wherein R^{3d*} is in each case independently selected from hydrogen or C₁₋₄alkyl.

In some embodiments X^{3a} is CR^{3a}, X^{3b} is CR^{3b}, and each of R^{3a}, R^{3b}, R^{3c}, and R^{3d} are hydrogen.

In some embodiments X^{3a} is CR^{3a}, X^{3b} is CR^{3b}, R^{3a} is F, Cl, Br, CN, OC₁₋₃alkyl, OC₁₋₃haloalkyl, or C₁₋₃alkyl, and each of R^{3b} R^{3C} and R^{3d} are hydrogen.

In some embodiments X^{3a} is CR^{3a}, X^{3b} is CR^{3b}, R^{3b} is F, Cl, Br, CN, OC₁₋₃alkyl, OC₁₋₃haloalkyl, or C₁₋₃alkyl, and each of R^{3a}, R^{3C}, and R^{3d} are hydrogen.

In some embodiments X^{3a} is CR^{3a}, X^{3b} is CR^{3b}, R^{3a} is F, Cl, Br, CN, OC₁₋₃alkyl, OC₁₋₃haloalkyl, or C₁₋₃alkyl, R^{3b} is F, Cl, Br, CN, OC₁₋₃alkyl, OC₁₋₃haloalkyl, or C₁₋₃alkyl, and R^{3c} and R^{3d} are both hydrogen.

In some embodiments X^{3a} is CR^{3a}, X^{3b} is CR^{3b}, R^{3a} is F, Cl, Br, CN, OC₁₋₃alkyl, OC₁₋₃haloalkyl, or C₁₋₃alkyl, R^{3d} is F, Cl, Br, CN, OC₁₋₃alkyl, OC₁₋₃haloalkyl, or C₁₋₃alkyl, and R^{3b} and R^{3c} are both hydrogen.

In some embodiments X^{3a} is CR^{3a}, X^{3b} is CR^{3b}, R^{3a} is F, Cl, Br, CN, OC₁₋₃alkyl, OC₁₋₃haloalkyl, or C₁₋₃alkyl, R^{3c} is F, Cl, Br, CN, OC₁₋₃alkyl, OC₁₋₃haloalkyl, and R^{3b} and R^{3d} are both hydrogen.

In some embodiments X^{3a} is CR^{3a}, X^{3b} is CR^{3b}, R^{3b} is F, Cl, Br, CN, OC₁₋₃alkyl, OC₁₋₃haloalkyl, or C₁₋₃alkyl, R^{3c} is F, Cl, Br, CN, OC₁₋₃alkyl, OC₁₋₃haloalkyl, or C₁₋₃alkyl, and R^{3a} and R^{3d} are both hydrogen.

In some embodiments X^{3a} is CR^{3a}, X^{3b} is CR^{3b}, R^{3b} is F, Cl, Br, CN, OC₁₋₃alkyl, OC₁₋₃haloalkyl, or C₁₋₃alkyl, R^{3d} is F, Cl, Br, CN, OC₁₋₃alkyl, OC₁₋₃haloalkyl, or C₁₋₃alkyl, and R^{3a} and R^{3c} are both hydrogen.

In some embodiments X^{3a} is N, X^{3b} is CR^{3b}, and each of R^{3b}, R^{3c}, and R^{3d} are hydrogen.

In some embodiments X^{3b} is N, X^{3a} is CR^{3a}, and each of R^{3a}, R^{3c}, and R^{3d} are hydrogen.

In some embodiments X^{3a} is N, X^{3b} is CR^{3b}, R^{3b} is F, Cl, Br, CN, OC₁₋₃alkyl, OC₁₋₃haloalkyl, or C₁₋₃alkyl, and R^{3c} and R^{3d} are both hydrogen.

In some embodiments X^{3a} is N, X^{3b} is CR^{3b}, R^{3c} is F, Cl, Br, CN, OC₁₋₃alkyl, OC₁₋₃haloalkyl, or C₁₋₃alkyl, and R^{3b} and R^{3d} are both hydrogen.

In some embodiments X^{3a} is N, X^{3b} is CR^{3b}, R^{3d} is F, Cl, Br, CN, OC₁₋₃alkyl, OC₁₋₃haloalkyl, or C₁₋₃alkyl, and R^{3b} and R^{3c} are both hydrogen.

In some embodiments X^{3b} is N, X^{3a} is CR^{3a}, R^{3a} is F, Cl, Br, CN, OC₁₋₃alkyl, OC₁₋₃haloalkyl, or C₁₋₃alkyl, and R^{3c} and R^{3d} are both hydrogen.

In some embodiments X^{3b} is N, X^{3a} is CR^{3a}, R^{3c} is F, Cl, Br, CN, OC₁₋₃alkyl, OC₁₋₃haloalkyl, or C₁₋₃alkyl, and R^{3a} and R^{3d} are both hydrogen.

In some embodiments X^{3b} is N, X^{3a} is CR^{3a}, R^{3d} is F, Cl, Br, CN, OC₁₋₃alkyl, OC₁₋₃haloalkyl, or C₁₋₃alkyl, and R^{3a} and R^{3c} are both hydrogen.

In some embodiments the A⁴ has the formula:

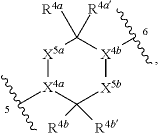

wherein
R^{4a} is F, Cl, Br, I, NO₂, CN, R^{4a*}, OR^{4a*}, N(R^{4a*})₂, SO₃R^{4a*}, SO₂R^{4a*}, SO₂N(R^{4a*})₂, C(O)R^{4a*}; C(O)OR^{4a*}, OC(O)R^{4a*}; C(O)N(R^{4a*})₂, N(R^{4a*})C(O)R^{4a*}, OC(O)N(R^{4a*})₂, N(R^{4a*})C(O)N(R^{4a*})₂, wherein R^{4a*} is in each case independently selected from hydrogen, C₁₋₈alkyl, aryl, C₁₋₈heteroaryl, C₃₋₈cycloalkyl, or C₁₋₈heterocyclyl;
R^{4a′} is F, Cl, Br, I, NO₂, CN, R^{4a′*}, OR^{4a′*}, N(R^{4a′*})₂, SO₃R^{4a′*}, SO₂R^{4a′*}, SO₂N(R^{4a′*})₂, C(O)R^{4a′*}; C(O)OR^{4a′*}, OC(O)R^{4a′*}; C(O)N(R^{4a′*})₂, N(R^{4a′*})C(O)R^{4a′*}, OC(O)N(R^{4a′*})₂, N(R^{4a′*})C(O)N(R^{4a′*})₂, wherein R^{4a′*} is in each case independently selected from hydrogen, C₁₋₈alkyl, aryl, C₁₋₈heteroaryl, C₃₋₈cycloalkyl, or C₁₋₈heterocyclyl;
wherein R^{4a} and R^{4a′} may together form an oxo or thiooxo;
R^{4b} is F, Cl, Br, I, NO₂, CN, R^{4b*}, OR^{4b*}, N(R^{4b*})₂, SO₃R^{4b*}, SO₂R^{4b*}, SO₂N(R^{4b*})₂, C(O)R^{4b*}; C(O)OR^{4b*}, OC(O)R^{4b*}; C(O)N(R^{4b*})₂, N(R^{4b*})C(O)R^{4b*}, OC(O)N(R^{4b*})₂, N(R^{4b*})C(O)N(R^{4b*})₂, wherein R^{4b*} is in each case independently selected from hydrogen, C₁₋₈alkyl, aryl, C₁₋₈heteroaryl, C₃₋₈cycloalkyl, or C₁₋₈heterocyclyl;
R^{4b′} is F, Cl, Br, I, NO₂, CN, R^{4b′*}, OR^{4b′*}, N(R^{4b′*})₂, SO₃R^{4b′*}, SO₂R^{4b′*}, SO₂N(R^{4b′*})₂, C(O)R^{4b′*}; C(O)OR^{4b′*}, OC(O)R^{4b′*}; C(O)N(R^{4b′*})₂, N(R^{4b′*})C(O)R^{4b′*}, OC(O)N(R^{4b′*})₂, N(R^{4b′*})C(O)N(R^{4b′*})₂, wherein R^{4b′*} is in each case independently selected from hydrogen, C₁₋₈alkyl, aryl, C₁₋₈heteroaryl, C₃₋₈cycloalkyl, or C₁₋₈heterocyclyl;
wherein R^{4b} and R^{4b′} may together form an oxo or thiooxo;
X^{4a} is N or CR^{4c}
X^{4b} is N or CR^{4d}
R^{4c} is F, Cl, Br, I, NO₂, CN, R^{4c*}, OR^{4c*}, N(R^{4c*})₂, SO₃R^{4c*}, SO₂R^{4c*}, SO₂N(R^{4c*})₂, C(O)R^{4c*}; C(O)OR^{4c*}, OC(O)R^{4c*}; C(O)N(R^{4c*})₂, N(R^{4c*})C(O)R^{4c*}, OC(O)N(R^{4c*})₂, N(R^{4c*})C(O)N(R^{4c*})₂, wherein R^{4c*} is in each case independently selected from hydrogen, C₁₋₈alkyl, aryl, C₁₋₈heteroaryl, C₃₋₈cycloalkyl, or C₁₋₈heterocyclyl;
R^{4d} is F, Cl, Br, I, NO₂, CN, R^{4d*}, OR^{4d*}, N(R^{4d*})₂, SO₃R^{4d*}, SO₂R^{4d*}, SO₂N(R^{4d*})₂, C(O)R^{4d*}; C(O)OR^{4d*}, OC(O)R^{4d*}; C(O)N(R^{4d*})₂, N(R^{4d*})C(O)R^{4d*}, OC(O)N(R^{4d*})₂, N(R^{4d*})C(O)N(R^{4d*})₂, wherein R^{4d*} is in each case independently selected from hydrogen, C₁₋₈alkyl, aryl, C₁₋₈heteroaryl, C₃₋₈cycloalkyl, or C₁₋₈heterocyclyl;
X^{5a} is null or CR^{4e}R^{4e′};
X^{5b} is null or CR^{4f}R^{4f′},
R^{4e} is F, Cl, Br, I, NO₂, CN, R^{4e*}, OR^{4e*}, N(R^{4e*})₂, SO₃R^{4e*}, SO₂R^{4e*}, SO₂N(R^{4e*})₂, C(O)R^{4e*}; C(O)OR^{4e*}, OC(O)R^{4e*}; C(O)N(R^{4e*})₂, N(R^{4e*})C(O)R^{4e*}, OC(O)N(R^{4e*})₂, N(R^{4e*})C(O)N(R^{4e*})₂, wherein R^{4e*} is in each case independently selected from hydrogen, C₁₋₈alkyl, aryl, C₁₋₈heteroaryl, C₃₋₈cycloalkyl, or C₁₋₈heterocyclyl;
R^{4e′} is F, Cl, Br, I, NO₂, CN, R^{4e′*}, OR^{4e′*}, N(R^{4e′*})₂, SO₃R^{4e′*}, SO₂R^{4e′*}, SO₂N(R^{4e′*})₂, C(O)R^{4e′*}; C(O)OR^{4e′*}, OC(O)R^{4e′*}; C(O)N(R^{4e′*})₂, N(R^{4e′*})C(O)R^{4e′*}, OC(O)N(R^{4e′*})₂, N(R^{4e′*})C(O)N(R^{4e′*})₂, wherein R^{4e′*} is in each case independently selected from hydrogen, C₁₋₈alkyl, aryl, C₁₋₈heteroaryl, C₃₋₈cycloalkyl, or C₁₋₈heterocyclyl;
wherein R^{4e} and R^{4e′} may together form an oxo or thiooxo;
R^{4f} is F, Cl, Br, I, NO₂, CN, R^{4f*}, OR^{4f*}, N(R^{4f*})₂, SO₃R^{4f*}, SO₂R^{4f*}, SO₂N(R^{4f*})₂, C(O)R^{4f*}; C(O)OR^{4f*}, OC(O)R^{4f*}; C(O)N(R^{4f*})₂, N(R^{4f*})C(O)R^{4f*}, OC(O)N(R^{4f*})₂, N(R^{4f*})C(O)N(R^{4f*})₂, wherein R^{4f*} is in each case independently selected from hydrogen, C₁₋₈alkyl, aryl, C₁₋₈heteroaryl, C₃₋₈cycloalkyl, or C₁₋₈heterocyclyl;
R^{4f′} is F, Cl, Br, I, NO₂, CN, R^{4f′*}, OR^{4f′*}, N(R^{4f′*})₂, SO₃R^{4f′*}, SO₂R^{4f′*}, SO₂N(R^{4f′*})₂, C(O)R^{4f′*}; C(O)OR^{4f′*}, OC(O)R^{4f′*}; C(O)N(R^{4f′*})₂, N(R^{4f′*})C(O)R^{4f′*}, OC(O)N(R^{4f′*})₂, N(R^{4f′*})C(O)N(R^{4f′*})₂, wherein R^{4f′*} is in each case independently selected from hydrogen, C₁₋₈alkyl, aryl, C₁₋₈heteroaryl, C₃₋₈cycloalkyl, or C₁₋₈heterocyclyl;
wherein R^{4f} and R^{4f′} may together form an oxo or thiooxo;
wherein any two or more of R^{4a}, R^{4a′}, R^{4b}, R^{4b′}, R^{4c}, R^{4d}, R^{4e}, R^{4e}′, R^{4f}, and R^{4f′} may together form a ring;
wherein wavy line 5 represents the point of attachment to A³ and wavy line 6 represents the point of attachment to R.

In some embodiments R^{4a} and R^{4a′} form an oxo, or wherein R^{4a} is F, Cl, Br, I, CN, R^{4a*}, OR^{4a*}, N(R^{4a*})₂, wherein R^{4a*} is in each case independently selected from hydrogen and C₁₋₈alkyl, and R^{4a′} together with another R group forms a ring, or is H.

In some embodiments R^{4b} and R^{4b′} form an oxo, or wherein R^{4b} is F, Cl, Br, I, CN, R^{4b*}, OR^{4b*}, N(R^{4b*})₂, wherein R^{4b*} is in each case independently selected from hydrogen and C₁₋₈alkyl, and R^{4b′} together with another R group forms a ring, or is H.

In some embodiments R^{4c}and R^{4e′} form an oxo, or wherein R^{4c} is F, Cl, Br, I, CN, R^{4c*}, OR^{4c*}, N(R^{4c*})₂, wherein R^{4c*} is in each case independently selected from hydrogen and C₁₋₈alkyl, and R^{4e′} together with another R group forms a ring, or is H.

In some embodiments R^{4d} and R^{4d′} form an oxo, or wherein R^{4d} is F, Cl, Br, I, CN, R^{4d*}, OR^{4d*}, N(R^{4d*})₂, wherein R^{4d*} is in each case independently selected from hydrogen and C₁₋₈alkyl, and R^{4d′} together with another R group forms a ring, or is H.

In some embodiments R^{4e} and R^{4e′} form an oxo, or wherein R^{4e} is F, Cl, Br, I, CN, R^{4e*}, OR^{4e*}, N(R^{4e*})₂, wherein R^{4e*} is in each case independently selected from hydrogen and C₁₋₈alkyl, and R^{4e′} together with another R group forms a ring, or is H.

In some embodiments R^{4f} and R^{4f′} form an oxo, or wherein R^{4f} is F, Cl, Br, I, CN, R^{4f*}, OR^{4f*}, N(R^{4f*})₂, wherein R^{4f*} is in each case independently selected from hydrogen and C₁₋₈alkyl, and R^{4f′} together with another R group forms a ring, or is H.

In some embodiments A⁴ has the formula:

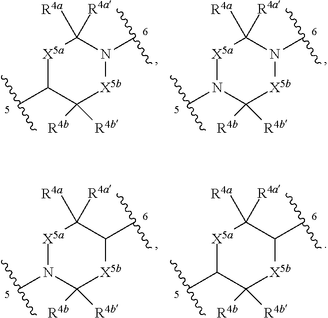

In some embodiments A⁴ has the formula:

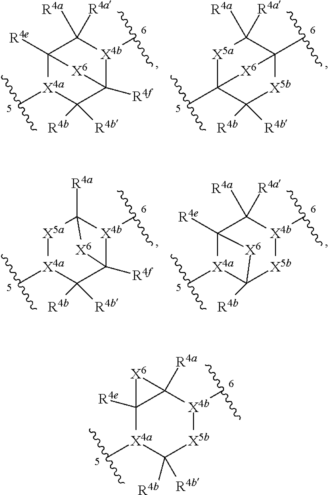

wherein X⁶ is Z², Z²—O, Z²—S, Z²—NRⁿ⁴, Z²—O—Z², Z²—S—Z², Z²—NRⁿ⁴—Z², wherein Z² is in each case null or a group having the formula:

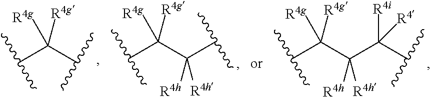

wherein R⁴ is selected from F, Cl, Br, I, NO₂, CN, R^{4g*}, OR^{4g*}, N(R^{4g*})₂, SO₃R^{4g*}, SO₂R^{4g*}, SO₂N(R^{4g*})₂, C(O)R^{4g*}; C(O)OR^{4g*}, OC(O)R^{4g*}; C(O)N(R^{4g*})₂, N(R^{4g*})C(O)R^{4g*}, OC(O)N(R^{4g*})₂, N(R^{4g*})C(O)N(R^{4g*})₂, wherein R^{4g*}is in each case independently selected from hydrogen, C₁₋₈alkyl, aryl, C₁₋₈heteroaryl, C₃₋₈cycloalkyl, or C₁₋₈heterocyclyl;
wherein R^{4g′} is selected from F, Cl, Br, I, NO₂, CN, R^{4g′*}, OR^{4g′*}, N(R^{4g′*})₂, SO₃R^{4g′*}, SO₂R^{4g′*}, SO₂N(R^{4g′*})₂, C(O)R^{4g′*}; C(O)OR^{4g′*}, OC(O)R^{4g′*}; C(O)N(R^{4g′*})₂, N(R^{4g′*})C(O)R^{4g′*}, OC(O)N(R^{4g′*})₂, N(R^{4g′*})C(O)N(R^{4g′*})₂, wherein R^{4g*} is in each case independently selected from hydrogen, C₁₋₈alkyl, aryl, C₁₋₈heteroaryl, C₃₋₈cycloalkyl, or C₁₋₈heterocyclyl;
or wherein R^{4g} and R^{4g′} together form an oxo, or thiooxo
wherein R^{4h} is selected from F, Cl, Br, I, NO₂, CN, R^{4h*}, OR^{4h*}, N(R^{4h*})₂, SO₃R^{4h*} SO₂R^{4h*}, SO₂N(R^{4h*})₂, C(O)R^{4h*}; C(O)OR^{4h*} OC(O)R^{4h*}; C(O)N(R^{4h*})₂, N(R^{4h*})C(O)R^{4h*}, OC(O)N(R^{4h*})₂, N(R^{4h*})C(O)N(R^{4h*})₂, wherein R^{4h*} is in each case independently selected from hydrogen, C₁₋₈alkyl, aryl, C₁₋₈heteroaryl, C₃₋₈cycloalkyl, or C₁₋₈heterocyclyl;
wherein R^{4h′} is selected from F, Cl, Br, I, NO₂, CN, R^{4h′*}, OR^{4h′*}, N(R^{4h′*})₂, SO₃R^{4h′*}, SO₂R^{4h′*}, SO₂N(R^{4h′*})₂, C(O)R^{4h′*}; C(O)OR^{4h′*}; OC(O)R^{4h′*}; C(O)N(R^{4h′*})₂, N(R^{4h′*})C(O)R^{4h′*}, OC(O)N(R^{4h′*})₂, N(R^{4h′*})C(O)N(R^{4h′*})₂, wherein R^{4h′*} is in each case independently selected from hydrogen, C₁₋₈alkyl, aryl, C₁₋₈heteroaryl, C₃₋₈cycloalkyl, or C₁₋₈heterocyclyl;
or wherein R^{4h} and R^{4h′} together form an oxo, or thiooxo
wherein R⁴ⁱ is selected from F, Cl, Br, I, NO₂, CN, R^{4i*}, OR^{4i*}, N(R^{4i*})₂, SO₃R^{4i*}, SO₂R^{4i*}, SO₂N(R^{4i*})₂, C(O)R^{4i*}; C(O)OR^{4i*}, OC(O)R^{4i*}; C(O)N(R^{4i*})₂, N(R^{4i*})C(O)R^{4i*}, OC(O)N(R^{4i*})₂, N(R^{4i*})C(O)N(R^{4i*})₂, wherein R^{4i′*} is in each case independently selected from hydrogen, C₁₋₈alkyl, aryl, C₁₋₈heteroaryl, C₃₋₈cycloalkyl, or C₁₋₈heterocyclyl;
wherein R^{4i′} is selected from F, Cl, Br, I, NO₂, CN, R^{4i′*}, OR^{4i′*}, N(R^{4i′*})₂, SO₃R^{4i′*}, SO₂R^{4i′*}, SO₂N(R^{4i′*})₂, C(O)R^{4i′*}; C(O)OR^{4i′*}, OC(O)R^{4i′*}; C(O)N(R^{4i′*})₂, N(R^{4i′*})C(O)R^{4i′*}, OC(O)N(R^{4i′*})₂, N(R^{4i′*})C(O)N(R^{4i′*})₂, wherein R^{4i′*} is in each case independently selected from hydrogen, C₁₋₈alkyl, aryl, C₁₋₈heteroaryl, C₃₋₈cycloalkyl, or C₁₋₈heterocyclyl;
or wherein R⁴ⁱ and R^{4i′} together form an oxo, or thiooxo

In some embodiments A⁴ is the formula:

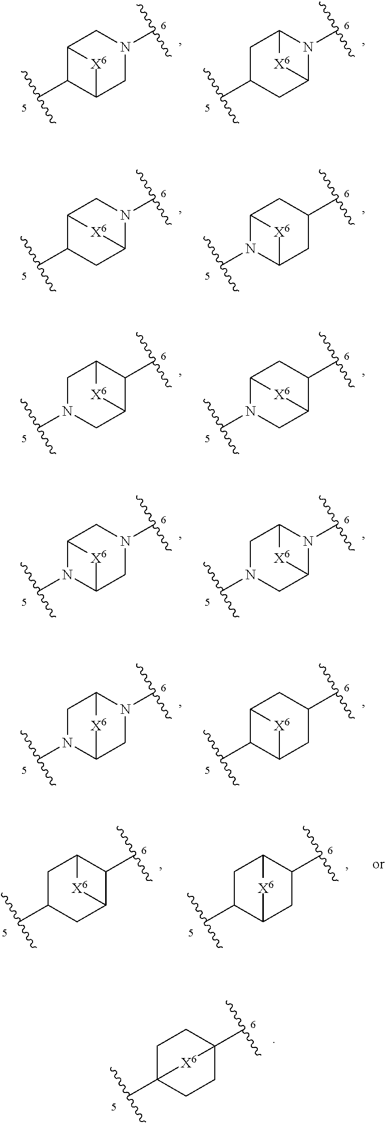

In some embodiments X⁶ is selected from null, CH₂, CH₂CH₂, CH₂CH₂CH₂, OC(═O), SC(═O), NHC(═O), N(CH₃)C(═O), CH₂C(═O), OCH₂, SCH₂, NHCH₂, N(CH₃)CH₂, CH₂C(═O)CH₂, CH₂OCH₂, CH₂SCH₂, CH₂NHCH₂, CH₂N(CH₃)CH₂, CH₂C(═O)CH₂, CH₂C(═O)O, CH₂C(═O)S, CH₂C(═O)NH, or CH₂C(═O)N(CH₃),

In some embodiments A⁴ has the formula:

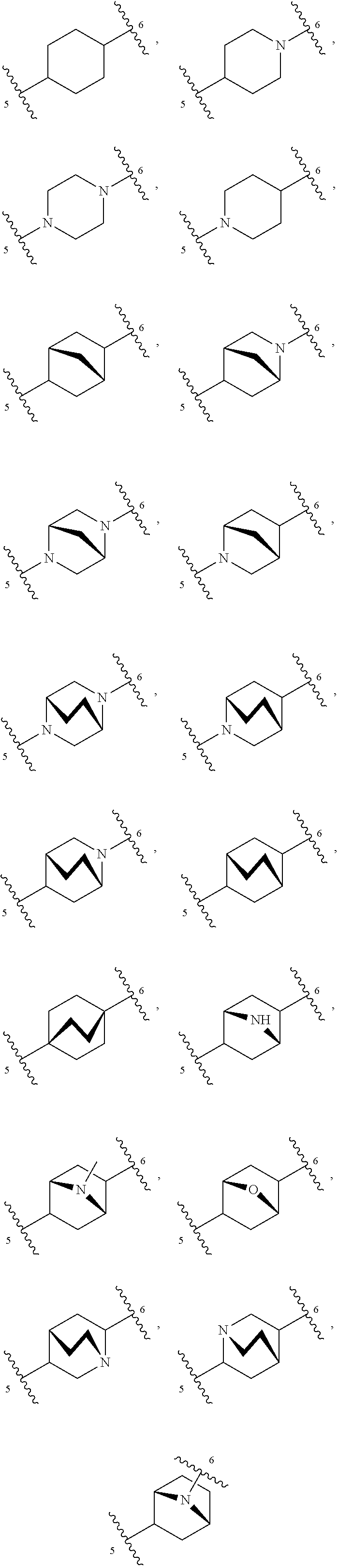

In further implementations, In certain implementations, A⁴, R^{a} and A³ together form a system having the formula:

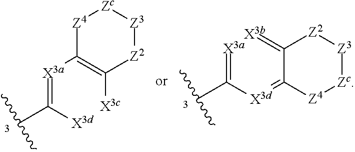

In certain implementations, A⁴, R^{a} and A³ together form a system having the formula:

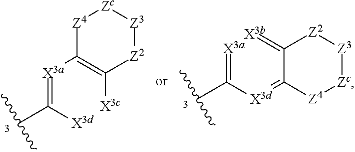

wherein Z² is null.

In certain implementations, A⁴, R^{a} and A³ together form a system having the formula:

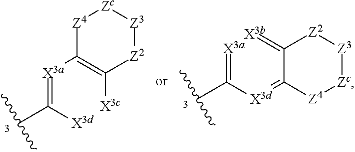

wherein Z³ is null.

In some embodiments A⁴, R^{a} and A³ together form a system having the formula:

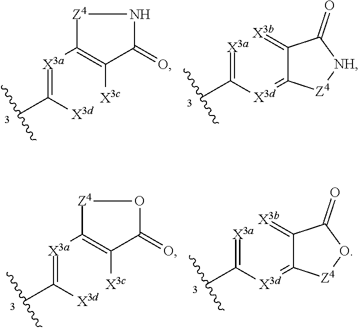

In some embodiments Z⁴ is C₁₋₆alkylene, preferably CH₂, CH₂CH₂, or CH₂CH₂CH₂.

In some embodiments X^{3a} is N, X^{3b} is CR^{3b}, and R^{3b} and Z^{t} together form a bond.

In some embodiments X^{3b} is N, X^{3c} is CR^{3c}, and R^{3c} and Z^{t} together form a bond.

In some embodiments X^{3a} is CR^{a}, X^{3b} is CR^{3b}, and R^{3b} and Z^{t} together form a bond.

In some embodiments X^{3b} is CR^{b}, X^{3c} is CR^{3c}, and R^{3c} and Z^{t} together form a bond.

In further implementations, A⁴, R^{a} and A³ together form a system having the formula:

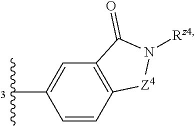

wherein R^{z4} is C₁₋₃alkyl, preferably CH₃, and Z⁴ is CH₂, CH₂CH₂, or CH₂CH₂CH₂.

In some embodiments R^{a} has the formula:

—Z³—Z^{c}—Z⁴—Z^{t},
wherein
Z³ is null
Z^{c} is null, —C(═O)—, —C(═O)O—, or —C(═O)NH—
Z⁴ is C₁₋₆alkylene,
Z^{t} is H, F, Cl, Br, I, CN, aryl, C₁₋₈heteroaryl, C₃₋₈cycloalkyl, C₁₋₈heterocyclyl, OR^{za}, N(R^{za})₂, C(═O)OR^{za}, C(═O)NR^{za*}, wherein R^{za*} is in each case independently selected from hydrogen, C₁₋₈alkyl, aryl, C₁₋₈heteroaryl, C₃₋₈cycloalkyl, or C₁₋₈heterocyclyl.

In some embodiments Z^{t} is OH.

In some embodiments Z^{t} is H.

In some implementations, A³, A⁴, and R^{a} together form a system having the formula:

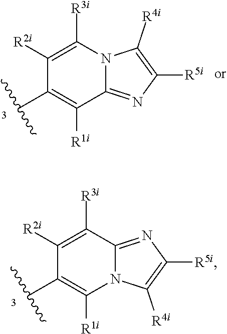

wherein
R¹ⁱ is F, Cl, Br, I, NO₂, CN, R^{1i*}, OR^{1i*}, N(R^{1i*})₂, SO₃R^{1i*}, SO₂R^{1i*}, SO₂N(R^{1i*})₂, C(O)R^{1i*}; C(O)OR^{1i*}, OC(O)R^{1i*}; C(O)N(Rui*)₂, N(R^{1i*})C(O)R^{1i*}, OC(O)N(R^{1i*})₂, N(R^{1i*})C(O)N(R^{1i*})₂, wherein R^{1i*} is in each case independently selected from hydrogen, C₁₋₈alkyl, aryl, C₁₋₈heteroaryl, C₃₋₈cycloalkyl, or C₁₋₈heterocyclyl;
R²ⁱ is F, Cl, Br, I, NO₂, CN, R^{2i*}, OR^{2i*}, N(R^{2i*})₂, SO₃R^{2i*}, SO₂R^{2i*}, SO₂N(R^{2i*})₂, C(O)R^{2i*}; C(O)OR^{2i*}, OC(O)R^{2i*}; C(O)N(R^{2i*})₂, N(R^{2i*})C(O)R^{2i*}, OC(O)N(R^{2i*})₂, N(R^{2i*})C(O)N(R^{2i*})₂, wherein R^{2i*} is in each case independently selected from hydrogen, C₁₋₈alkyl, aryl, C₁₋₈heteroaryl, C₃₋₈cycloalkyl, or C₁₋₈heterocyclyl;
R³ⁱ is F, Cl, Br, I, NO₂, CN, R^{3i*}, OR^{3i*}, N(R^{3i*})₂, SO₃R^{3i*}, SO₂R^{3i*}, SO₂N(R^{3i*})₂, C(O)R^{3i*}; C(O)OR^{3i*}, OC(O)R^{3i*}; C(O)N(R^{3i*})₂, N(R^{3i*})C(O)R^{3i*}, OC(O)N(R^{3i*})₂, N(R^{3i*})C(O)N(R^{3i*})₂, wherein R^{3i*} is in each case independently selected from hydrogen, C₁₋₈alkyl, aryl, C₁₋₈heteroaryl, C₃₋₈cycloalkyl, or C₁₋₈heterocyclyl;
R⁴ⁱ is F, Cl, Br, I, NO₂, CN, R^{4i*}, OR^{4i*}, N(R^{4i*})₂, SO₃R^{4i*}, SO₂R^{4i*}, SO₂N(R^{4i*})₂, C(O)R^{4i*}; C(O)OR^{4i*}, OC(O)R^{4i*}; C(O)N(R^{4i*})₂, N(R^{4i*})C(O)R^{4i*}, OC(O)N(R^{4i*})₂, N(R^{4i*})C(O)N(R^{4i*})₂, wherein R^{4i*} is in each case independently selected from hydrogen, C₁₋₈alkyl, aryl, C₁₋₈heteroaryl, C₃₋₈cycloalkyl, or C₁₋₈heterocyclyl;
R⁵ⁱ is F, Cl, Br, I, NO₂, CN, R^{5i*}, OR^{5i*}, N(R^{5i*})₂, SO₃R^{5i*}, SO₂R^{5i*}, SO₂N(R^{5i*})₂, C(O)R^{5i*}; C(O)OR^{5i*}, OC(O)R^{5i*}; C(O)N(R^{5i*})₂, N(R^{5i*})C(O)R^{5i*}, OC(O)N(R^{5i*})₂, N(R^{5i*})C(O)N(R^{5i*})₂, wherein R^{5i*} is in each case independently selected from hydrogen, C₁₋₈alkyl, aryl, C₁₋₈heteroaryl, C₃₋₈cycloalkyl, or C₁₋₈heterocyclyl;
wherein any two or more of R¹ⁱ, R²ⁱ, R³ⁱ, R⁴ⁱ, and R⁵ⁱ can together form a ring.

In certain implementations, R¹ⁱ, R²ⁱ, and R³ⁱ are each hydrogen. In certain implementations, R⁴ⁱ and R⁵ⁱ are independently selected from H, C₁₋₆alkyl, C₃₋₈cycloalkyl or R⁴ⁱ and R⁵ⁱ together form a 5 atom, 6 atom, or 7 atom carbocyclic or heterocyclic ring. In certain implementations, R⁴ⁱ and R⁵ⁱ together form a carbocyclic ring, in certain implementations, R⁴ⁱ and R⁵ⁱ together an aromatic ring. In further implementation R⁴ⁱ and R⁵ⁱ together form a heterocyclic ring. In some implementations R⁴ⁱ and R⁵ⁱ are each CH₃. In some implementations R⁴ⁱ is C₁₋₃alkyl and R⁵ⁱ is H. In some implementations R⁴ⁱ is H and R⁵ⁱ is C₁₋₃alkyl. In some implementations R⁴ⁱ is isopropyl and R⁵ⁱ is H. In some implementations R⁴ⁱ is H and R⁵ⁱ is isopropyl.

In some implementations, A³, A⁴, and R^{a} together form a system having the formula:

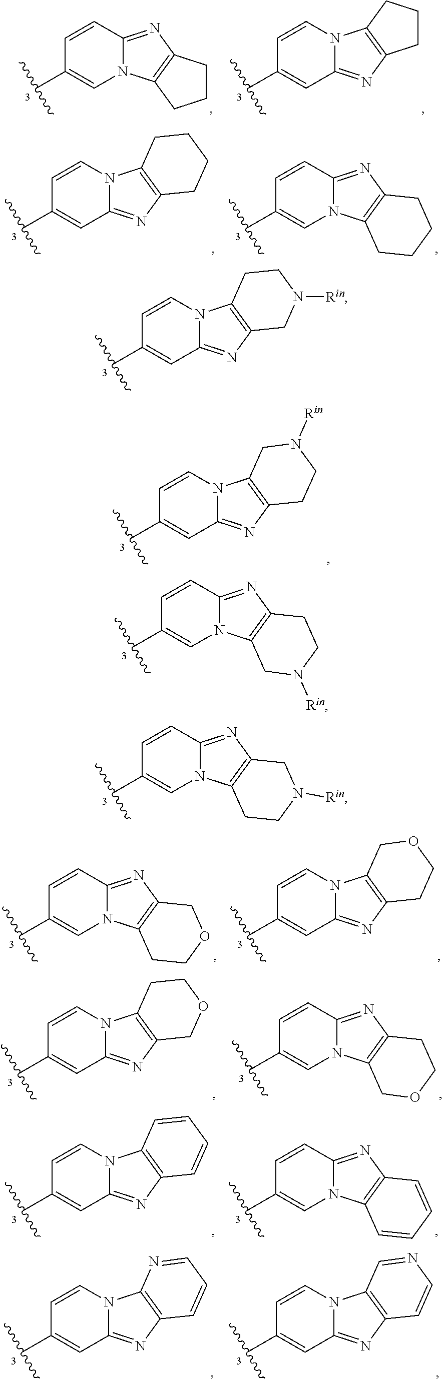

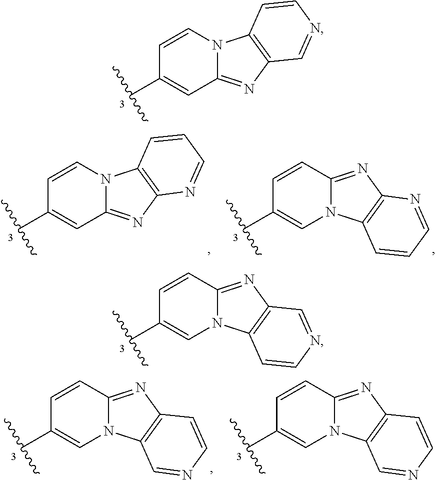

In some implementations, A³, A⁴, and R^{a} together form a system having the formula:

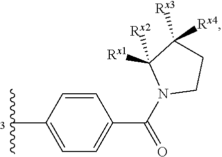

wherein R^{x1} and R^{x2} are independently selected from H, F, Cl, Br, or CH₃, and R^{x3} and R^{x4} are independently selected from H, F, Cl, Br, or CH₃, or one of R^{x3} and R^{x4} is OH and the other is H or CH₃. In some implementations, R^{x1} and R^{x2} are each H, and R^{x3} and R^{x4} are F. In some implementations, R^{x1} is CH₃, and each of R^{x2}, R^{x3}, and R^{x4} are H. In some implementations, R^{x2} is CH₃, and each of R^{x1}, R^{x3}, and R^{x4} are H. In some implementations, R^{x3} is F, and each of R^{x1}, R^{x2}, and R^{x4} are H. In some implementations, R^{x4} is F, and each of R^{x1}, R^{x2}, and R^{x3} are H.

In some embodiments the compound has the structure:

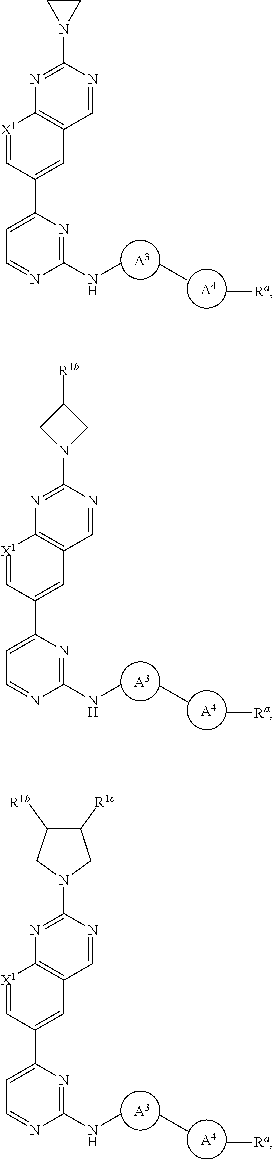

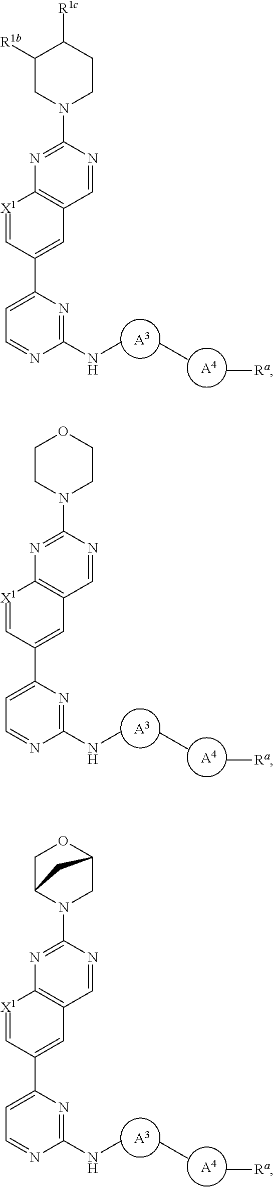

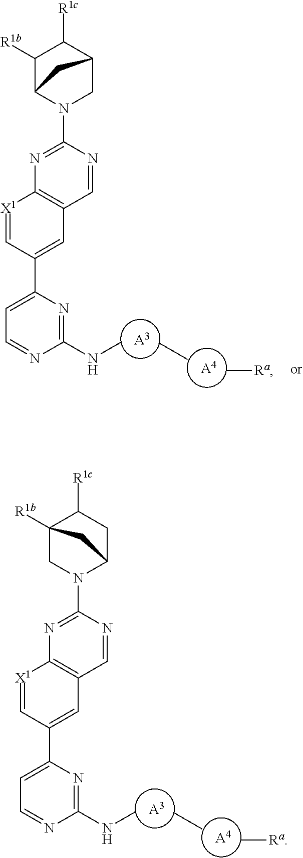

In some embodiments X¹ is N or CH.

In some embodiments the compound has the structure:

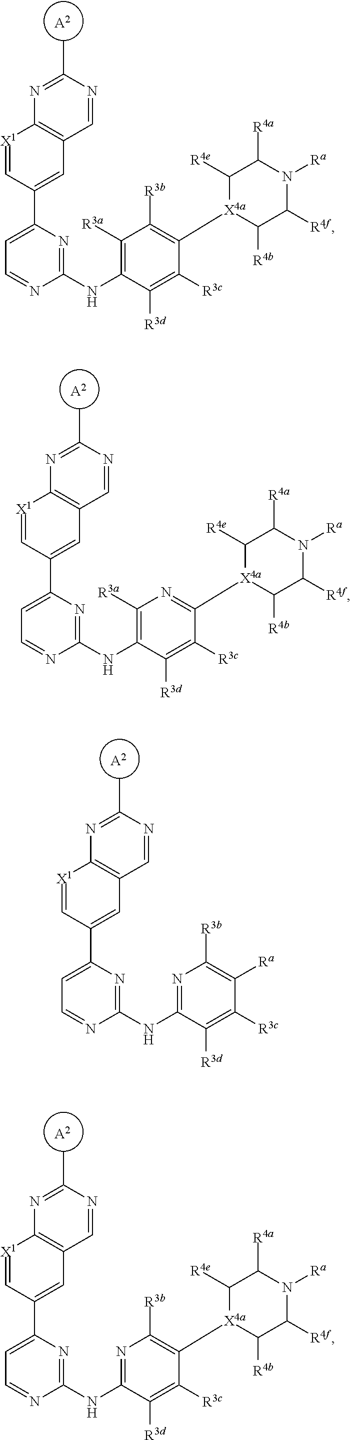

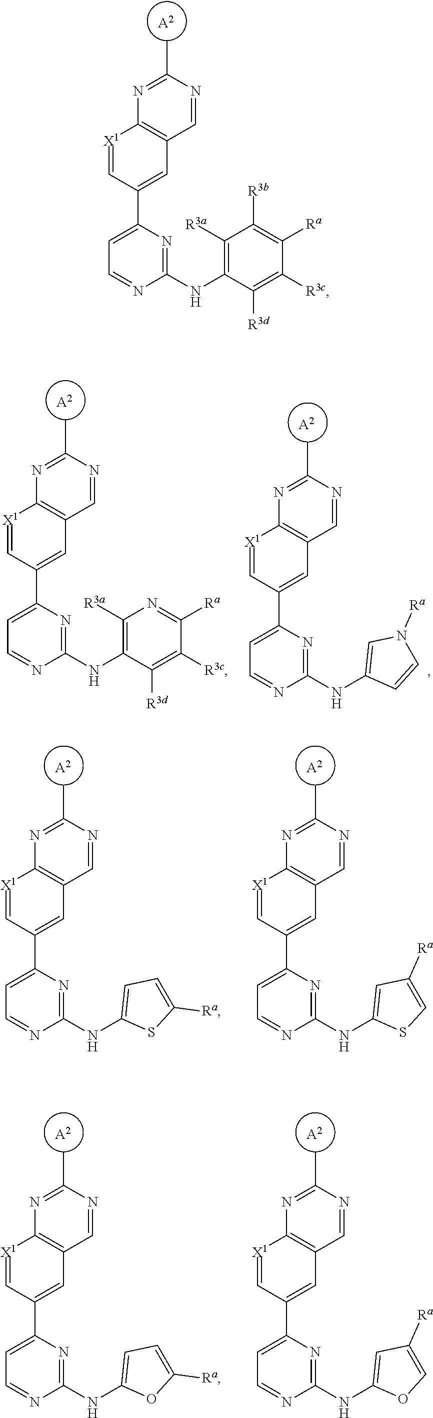

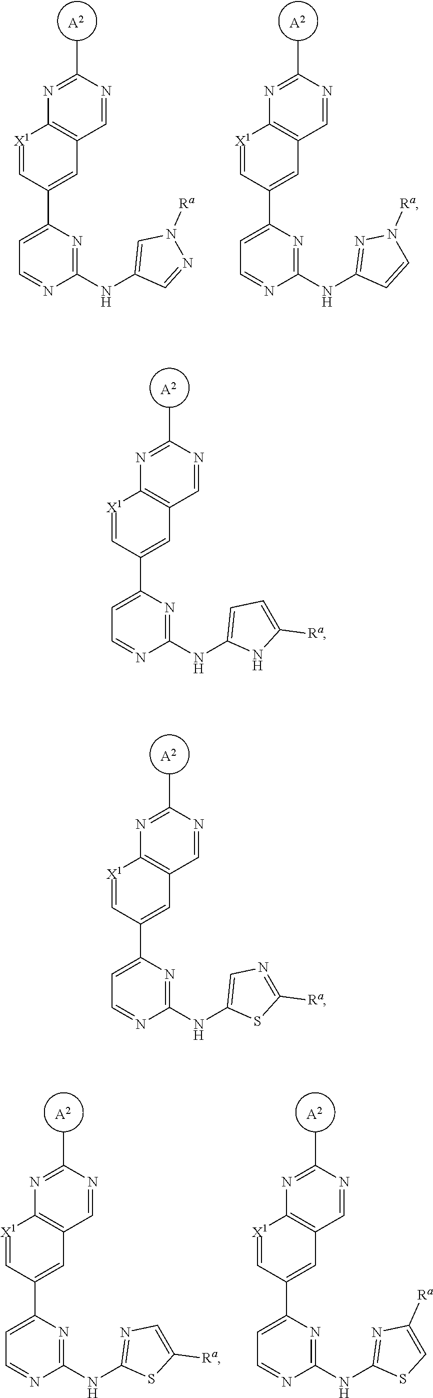

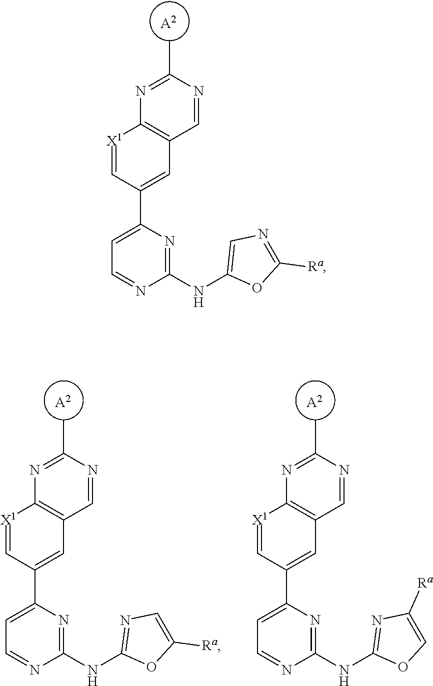

In certain embodiments, R^{a} is C₃₋₆carbocyclyl, C₁₋₆heterocyclyl, or C(═O)NHC₁₋₆alkyl. Z^{c}—H, Z^{c}—(CH₂)₂OH, Z^{c}—(CH₂)₃OH, Z^{c}—(CH₂)₄₀H, Z^{c}—(CH₂)₅OH, Z^{c}—(CH₂)₆OH, wherein Z^{c} is null, C(═O), C(═O)NH, or C(═O)O. In some implementations, R^{a} is C₃₋₆carbocyclyl or C₁₋₆heterocyclyl optionally substituted with OH, —CH₂OH, —(CH₂)₂OH, —(CH₂)₃₀H.

R^{3a} is H, F, Cl, Br, CN, OC₁₋₃alkyl, OC₁₋₃haloalkyl, or C₁₋₃alkyl;
R^{3b} is H, F, Cl, Br, CN, OC₁₋₃alkyl, OC₁₋₃haloalkyl, or C₁₋₃alkyl;
R^{3C} is H, F, Cl, Br, CN, OC₁₋₃alkyl, OC₁₋₃haloalkyl, or C₁₋₃alkyl;
R^{3d} is H, F, Cl, Br, CN, OC₁₋₃alkyl, OC₁₋₃haloalkyl, or C₁₋₃alkyl; In some embodiments R^{3a} is F, Cl, Br, CN, OC₁₋₈alkyl, OC₁₋₃haloalkyl, or C₁₋₃alkyl, is F, Cl, Br, CN, OC₁₋₈alkyl, OC₁₋₃haloalkyl, or C₁₋₃alkyl, and R^{3b}, R^{3c}, and R^{3d} are each hydrogen.

In some embodiments R^{3b} is F, Cl, Br, CN, OC₁₋₃alkyl, OC₁₋₃haloalkyl, or C₁₋₃alkyl, is F, Cl, Br, CN, OC₁₋₈alkyl, OC₁₋₃haloalkyl, or C₁₋₃alkyl, and R^{3a}, R^{3c}, and R^{3d} are each hydrogen.

In some embodiments R^{3C} is F, Cl, Br, CN, OC₁₋₃alkyl, OC₁₋₃haloalkyl, or C₁₋₃alkyl, is F, Cl, Br, CN, OC₁₋₃alkyl, OC₁₋₃haloalkyl, or C₁₋₃alkyl, and R^{3a}, R^{3b}, and R^{3d} are each hydrogen.

In some embodiments R^{3d} is F, Cl, Br, CN, OC₁₋₃alkyl, OC₁₋₃haloalkyl, or C₁₋₃alkyl, is F, Cl, Br, CN, OC₁₋₃alkyl, OC₁₋₃haloalkyl, or C₁₋₃alkyl, and R^{3a}, R^{3b}, and R^{3c} are each hydrogen.

In some embodiments X^{4a} is CH or N.

In some embodiments R^{4a} is hydrogen.

In some embodiments R^{4b} is hydrogen.

In some embodiments R^{4c} is hydrogen.

In some embodiments R^{4d} is hydrogen.

In some embodiments R^{4e} and R^{4f} together form a CH₂, O, NH, NCH₃, or CH₂CH₂.

In some implementations, A³, A⁴, and R^{a} together form a system having the formula:

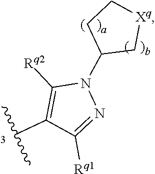

wherein
X^{q} is CH₂, O, or NR^{q3}
R^{q1} is H, F, Cl, Br, C₁₋₃alkyl, OC₁₋₃alkyl,
R^{q2} is H, F, Cl, Br, C₁₋₃alkyl, OC₁₋₃alkyl,
R^{q3} is H, (CH₂)_{c}OH or C(═O)OC₁₋₃alkyl,
a is 0, 1, or 2,
b is 1, 2, or 3, and
c is 1, 2, or 3.

In certain implementations, one of R^{q1} and R^{q2} is F, and the other is H. In some implementations, R^{q2} is H and R^{q1} is F. In certain implementations, X is NR^{g3}, and R^{q3} is H, (CH₂)_{c}OH or C(═O)OCH₃. In certain implementations, a is 1, b is 2, and c is 2; in other implementations, a is 0, b is 1, and c is 2. In certain implementations, R^{q3} is H or CH₂CH₂OH.

The following compounds are within the scope of the invention and have STK17A inhibitory activity include the following: (in all cases n is 1, 2, 3, 4, or 5; m=0, 1, or 2; R═H, C₁₋₈alkyl, C₃₋₈cycloalkyl, C₁₋₈heterocyclyl; R₁=H, F, Cl, Br, I, CN, C₁₋₈alkyl, OC₁₋₈alkyl:

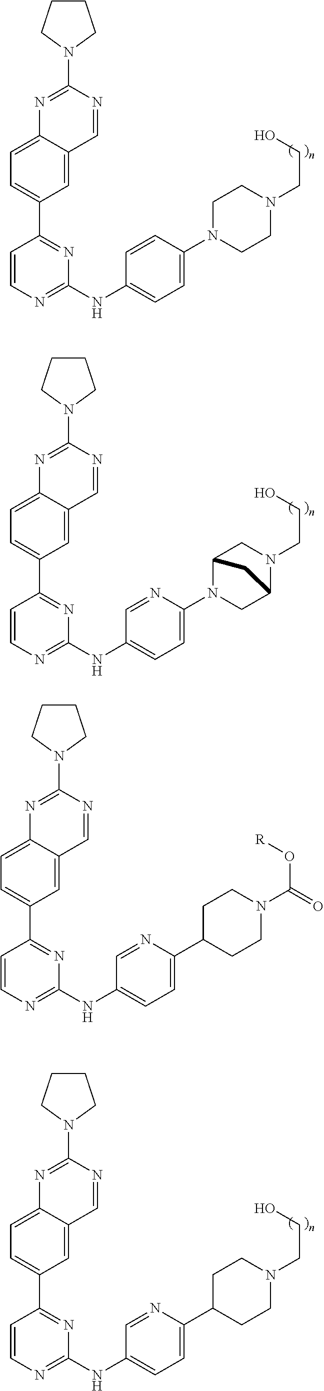

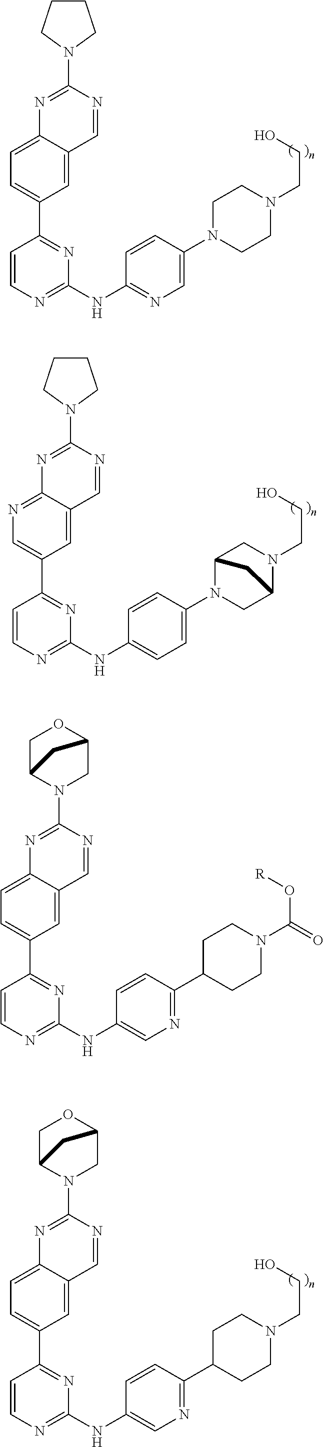

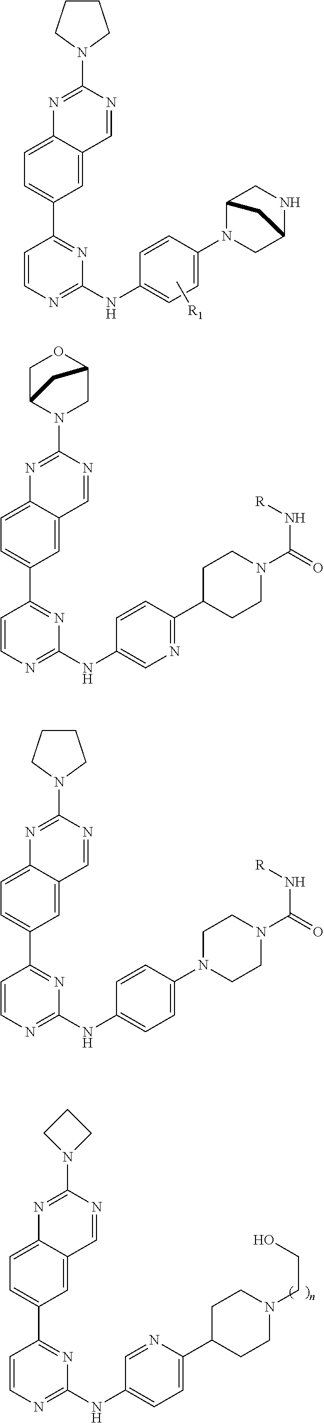

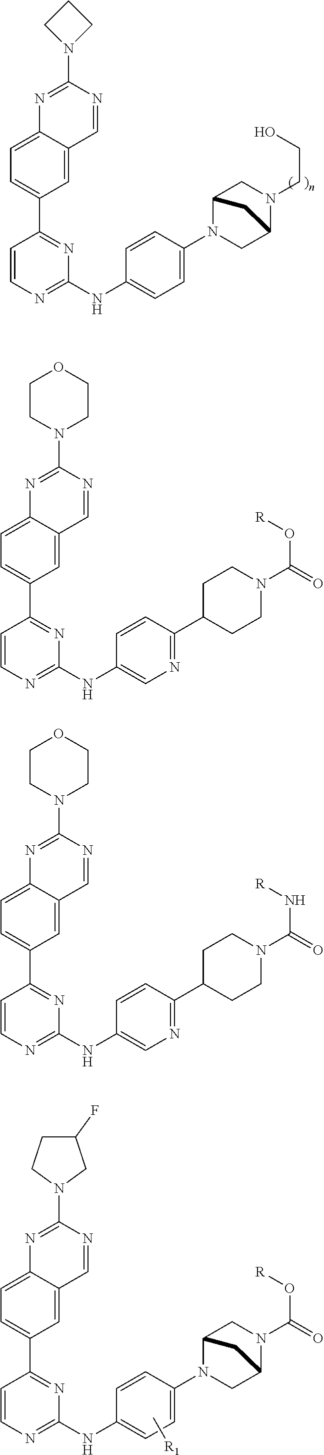

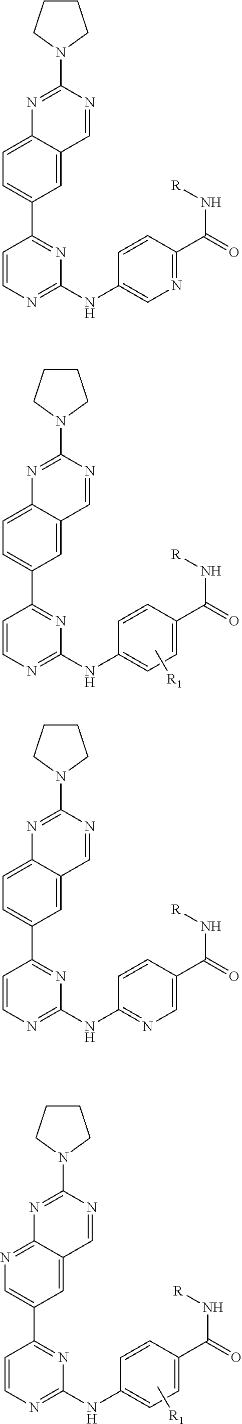

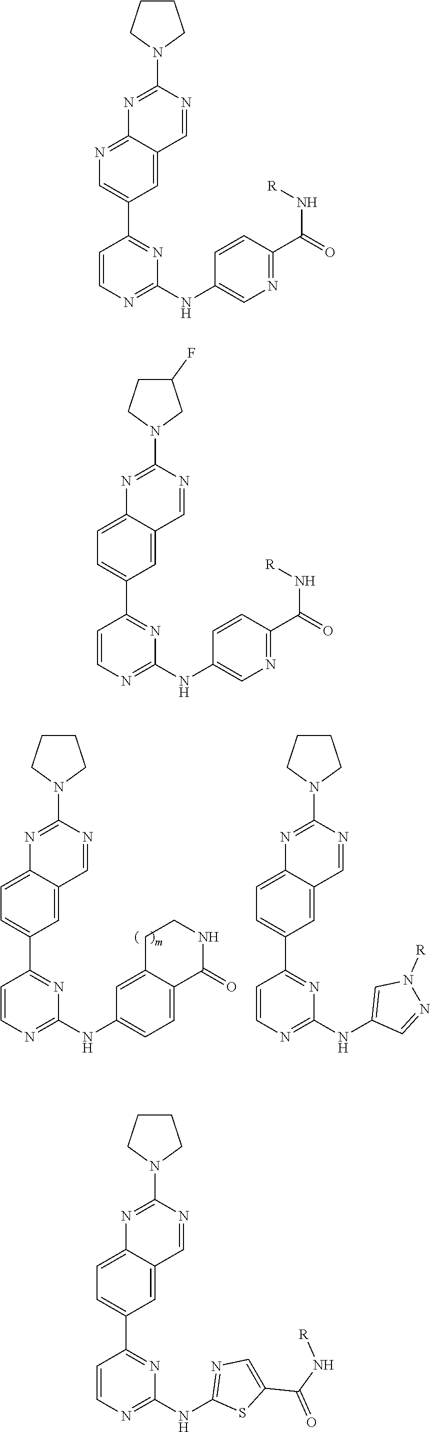

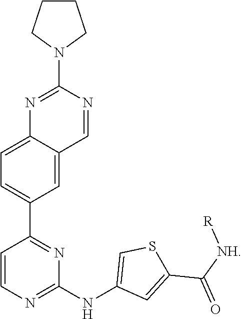

wherein R is C₃₋₆carbocyclyl, C₁₋₆heterocyclyl, or C(═O)NHC₁₋₆alkyl, Z^{c}—H, Z^{c}—(CH₂)₂OH, Z^{c}—(CH₂)₃OH, Z^{c}—(CH₂)₄OH, Z^{c}—(CH₂)₅OH, Z^{c}—(CH₂)₆OH, wherein Z^{c} is null, C(═O), C(═O)NH, or C(═O)O. In some implementations, R^{a} is C₃₋₆carbocyclyl or C₁₋₆heterocyclyl optionally substituted with OH, —CH₂OH, —(CH₂)₂OH, —(CH₂)₃OH.

The compounds disclosed herein may be formulated in pharmaceutical compositions for administration to a subject. Exemplary compositions will include at least one pharmaceutically acceptable excipient A pharmaceutical composition comprising a compound according to any preceding claim and at least one pharmaceutical excipient. Such compositions include, but are not limited to, unit dosage forms including tablets, capsules (filled with powders, pellets, beads, mini-tablets, pills, micro-pellets, small tablet units, multiple unit pellet systems (MUPS), disintegrating tablets, dispersible tablets, granules, and microspheres, multiparticulates), sachets (filled with powders, pellets, beads, mini-tablets, pills, micro-pellets, small tablet units, UPS, disintegrating tablets, dispersible tablets, granules, and microspheres, multiparticulates), powders for reconstitution, transdermal patches and sprinkles, however, other dosage forms such as controlled release formulations, lyophilized formulations, modified release formulations, delayed release formulations, extended release formulations, pulsatile release formulations, dual release formulations and the like. Liquid or semisolid dosage form (liquids, suspensions, solutions, dispersions, ointments, creams, emulsions, microemulsions, sprays, patches, spot-on), injection preparations, parenteral, topical, inhalations, buccal, nasal etc. may also be envisaged under the ambit of the invention.

Suitable excipients may be used for formulating the dosage forms according to the present invention such as, but not limited to, surface stabilizers or surfactants, viscosity modifying agents, polymers including extended release polymers, stabilizers, disintegrants or super disintegrants, diluents, plasticizers, binders, glidants, lubricants, sweeteners, flavoring agents, anti-caking agents, opacifiers, anti-microbial agents, antifoaming agents, emulsifiers, buffering agents, coloring agents, carriers, fillers, anti-adherents, solvents, taste-masking agents, preservatives, antioxidants, texture enhancers, channeling agents, coating agents or combinations thereof.

The compounds disclosed herein may be administered by a number of different routes. For instance, the compounds may be administered orally, topically, transdermally, intravenously, subcutaneously, by inhalation, or by intracerebroventricular delivery.

In some embodiments, the compounds disclosed herein may be formulated as nanoparticles. The nanoparticles may have an average particle size from 1-1,000 nm, preferably 10-500 nm, and even more preferably from 10-200 nm.

The compounds may be administered to a patient systemically, e.g., by oral or intravenous administration, topically, i.e., by application of a cream, lotion or the like, or locally, e.g., by direct perfusion of a composition containing the compound to a target tissue.

The disclosed compounds have STK17A inhibitory activity and as such may be effectively used for the treatment of proliferative disorders, including cancer and similar diseases. The compounds may be used to treat cancers characterized by one or more solid tumors. In other embodiments, the proliferative disorder is a blood cancer. In some embodiments, the proliferative disorder is myelodysplastic syndrome. In some embodiments, the proliferative disorder is leukemia.

In some instances the compounds may be used to treat proliferative disorder such as acute myeloid leukemia, chronic myeloid leukemia, acute lymphoblastic leukemia, multiple myeloma, mixed lineage leukemia, brain tumor, glioblastoma, or lymphoma.

In certain embodiments, the compounds may be used to treat bone cancer.

In certain embodiments, the compounds may be used to treat glioblastoma.

In certain embodiments, the compounds may be used to treat osteosarcoma, Ewing sarcoma, chondrosarcoma, undifferentiated pleomorphic sarcoma, fibrosarcoma, chordoma, or malignant giant cell tumor.

In certain embodiments, the compounds may be used to treat an osteosarcoma such as an osteoblastic cancer, chrondroblastic cancer, fibroblastic cancer, small cell cancer, telangiectatic cancer.

In other embodiments, the compounds may be used to treat other cancers. Exemplary cancers include acute lymphoblastic leukemia (ALL), acute myeloid leukemia (AML), cancer in adrenocortical carcinoma, adrenal cortex cancer, AIDS-related cancers, Kaposi sarcoma, AIDS-related lymphoma, primary CNS lymphoma, anal cancer, appendix cancer, carcinoid tumors, astrocytomas, atypical teratoid/rhabdoid tumor, basal cell carcinoma, skin cancer (nonmelanoma), bile duct cancer, extrahepatic bladder cancer, bladder cancer, bone cancer (includes Ewing sarcoma and osteosarcoma and malignant fibrous histiocytoma), brain tumors, breast cancer, bronchial tumors, Burkitt lymphoma (non-Hodgkin), carcinoid tumor, cardiac (heart) tumors, atypical teratoid/rhabdoid tumor, embryonal tumors, germ cell tumors, lymphoma, primary—cervical cancer, cholangiocarcinoma, chordoma, chronic lymphocytic leukemia (CLL), chronic myelogenous leukemia (CML), chronic myeloproliferative neoplasms, colorectal cancer, colorectal cancer, craniopharyngioma, cutaneous T-cell lymphoma, ductal carcinoma in situ (DCIS), embryonal tumors, central nervous system, endometrial cancer, ependymoma, esophageal, esthesioneuroblastoma, extracranial germ cell tumor, extragonadal germ cell tumor, eye cancer, intraocular melanoma, retinoblastoma, fallopian tube cancer, fibrous histiocytoma of bone, malignant, and osteosarcoma, gallbladder cancer, gastric (stomach) cancer, gastric (stomach) cancer, gastrointestinal carcinoid tumor, gastrointestinal stromal tumors (GIST), gastrointestinal stromal tumors (GIST), germ cell tumors, central nervous system, extracranial, extragonadal, ovarian testicular, gestational trophoblastic disease, gliomas, hairy cell leukemia, head and neck cancer, heart tumors, hepatocellular (liver) cancer, histiocytosis, Langerhans Cell, Hodgkin's lymphoma, hypopharyngeal cancer, intraocular melanoma, islet cell tumors, pancreatic neuroendocrine tumors, Kaposi sarcoma, kidney—langerhans cell histiocytosis, laryngeal cancer, laryngeal cancer and papillomatosis, leukemia, lip and oral cavity cancer, liver cancer (primary), lung cancer, lung cancer, lymphoma—macroglobulinemia, Waldenström-Non-Hodgkin lymphoma, male breast cancer, malignant fibrous histiocytoma of bone and osteosarcoma, melanoma, intraocular (eye), Merkel cell carcinoma, mesothelioma, malignant, mesothelioma, metastatic squamous neck cancer with occult primary, midline tract carcinoma involving NUT gene, mouth cancer, multiple endocrine neoplasia syndromes, multiple myeloma/plasma cell neoplasms, mycosis fungoides, myelodysplastic syndromes, myelodysplastic/myeloproliferative neoplasms and chronic myeloproliferative neoplasms, myelogenous leukemia, chronic (CML), myeloid leukemia, acute (AML), nasal cavity and paranasal sinus cancer, nasopharyngeal cancer, nasopharyngeal cancer, neuroblastoma, non-hodgkin lymphoma, non-small cell lung cancer, oral cancer, lip and oral cavity cancer and oropharyngeal cancer, osteosarcoma and malignant fibrous histiocytoma of bone, ovarian cancer, pancreatic cancer and pancreatic neuroendocrine tumors (islet cell tumors), papillomatosis, paraganglioma, paraganglioma, paranasal sinus and nasal cavity cancer, parathyroid cancer, penile cancer, pharyngeal cancer, pheochromocytoma, pheochromocytoma, pituitary tumor, plasma cell neoplasm/multiple myeloma, pleuropulmonary blastoma, pregnancy and breast cancer, primary central nervous system (CNS) lymphoma, primary peritoneal cancer, prostate cancer, rectal cancer, renal cell (kidney) cancer, retinoblastoma, rhabdomyosarcoma, salivary gland cancer, salivary gland tumors, Ewing sarcoma, Kaposi sarcoma, osteosarcoma, rhabdomyosarcoma, uterine sarcoma, vascular tumors, Sézary syndrome, skin cancer, small cell lung cancer, small intestine cancer, soft tissue sarcoma, squamous cell carcinoma, squamous neck cancer with occult primary, metastatic, stomach (gastric) cancer, stomach (gastric) cancer, T-cell lymphoma, cutaneous, testicular cancer, throat cancer, thymoma and thymic carcinoma, thyroid cancer, ureter and renal pelvis, transitional cell cancer, urethral cancer, uterine cancer, endometrial and uterine sarcoma, vaginal cancer, vaginal cancer, vascular tumors, vulvar cancer, Waldenstrom Macroglobulinemia, Wilms Tumor.

In the field of medical oncology it is normal practice to use a combination of different forms of treatment to treat each patient with cancer. In medical oncology the other component(s) of such conjoint treatment in addition to compositions of the present invention may be, for example, surgery, radiotherapy, chemotherapy, signal transduction inhibitors and/or monoclonal antibodies. As such, the compounds disclosed herein may be administered as part of a combination treatment regime, for instance prior to or following surgery or prior to or following radiation treatment.

In some embodiments, the compounds disclosed herein may be administered in combination with one or more anticancer agents for example mitotic inhibitors, alkylating agents, anti-metabolites, antisense DNA or RNA, intercalating antibiotics, growth factor inhibitors, signal transduction inhibitors, cell cycle inhibitors, enzyme inhibitors, retinoid receptor modulators, proteasome inhibitors, topoisomerase inhibitors, biological response modifiers, anti-hormones, angiogenesis inhibitors, cytostatic agents anti-androgens, targeted antibodies, HMG-CoA reductase inhibitors, and prenyl-protein transferase inhibitors.

Exemplary anti-cancer agents include nucleoside analogues, antifolates, antimetabolites, topoisomerase I inhibitor, anthracyclines, podophyllotoxins, taxanes, vinca alkaloids, alkylating agents, platinum compounds, proteasome inhibitors, nitrogen mustards & oestrogen analogue, monoclonal antibodies, tyrosine kinase inhibitors, mTOR inhibitors, retinoids, immunomodulatory agents, histone deacetylase inhibitors, and combinations thereof.

In certain embodiments, the anti-cancer agent is selected from one or more of abiraterone acetate, methotrexate, paclitaxel albumin-stabilized nanoparticle, brentuximab vedotin, ado-trastuzumab emtansine, doxorubicin hydrochloride, afatinib dimaleate, everolimus, netupitant, palonosetron hydrochloride, imiquimod, aldesleukin, alectinib, alemtuzumab, melphalan hydrochloride, melphalan, pemetrexed disodium, chlorambucil, aminolevulinic acid, anastrozole, aprepitant, pamidronate disodium, exemestane, nelarabine, arsenic trioxide, ofatumumab, asparaginase Erwinia chrysanthemi, atezolizumab, bevacizumab, axitinib, azacitidine, carmustine, belinostat, bendamustine hydrochloride, bevacizumab, bexarotene, tositumomab, bicalutamide, bleomycin, blinatumomab, blinatumomab, bortezomib, bosutinib, busulfan, cabazitaxel, cabozantinib, alemtuzumab, irinotecan hydrochloride, capecitabine, fluorouracil, carboplatin, carfilzomib, bicalutamide, lomustine, ceritinib, daunorubicin hydrochloride, cetuximab, chlorambucil, cyclophosphamide, clofarabine, cobimetinib, dactinomycin, cobimetinib, crizotinib, ifosfamide, ramucirumab, cytarabine, dabrafenib, dacarbazine, decitabine, daratumumab, dasatinib, daunorubicin hydrochloride, decitabine, efibrotide sodium, defibrotide sodium, degarelix, denileukin diftitox, denosumab, dexamethasone, dexrazoxane hydrochloride, dinutuximab, docetaxel, doxorubicin hydrochloride, dacarbazine, rasburicase, epirubicin hydrochloride, elotuzumab, oxaliplatin, eltrombopag olamine, aprepitant, elotuzumab, enzalutamide, epirubicin hydrochloride, cetuximab, eribulin mesylate, vismodegib, erlotinib hydrochloride, etoposide, raloxifene hydrochloride, melphalan hydrochloride, toremifene, panobinostat, fulvestrant, letrozole, filgrastim, fludarabine phosphate, flutamide, methotrexate, pralatrexate, recombinant hpv quadrivalent vaccine, recombinant hpv nonavalent vaccine, obinutuzumab, gefitinib, gemcitabine hydrochloride, gemtuzumab ozogamicin, afatinib dimaleate, imatinib mesylate, glucarpidase, goserelin acetate, eribulin mesylate, trastuzumab, topotecan hydrochloride, palbociclib, ibritumomab tiuxetan, ibrutinib, ponatinib hydrochloride, idarubicin hydrochloride, idelalisib, imiquimod, axitinib, recombinant interferon alfa-2b, tositumomab, ipilimumab, gefitinib, romidepsin, ixabepilone, ixazomib citrate, ruxolitinib phosphate, cabazitaxel, ado-trastuzumab emtansine, palifermin, pembrolizumab, lanreotide acetate, lapatinib ditosylate, lenalidomide lenvatinib mesylate, leuprolide acetate, olaparib, vincristine sulfate, procarbazine hydrochloride, mechlorethamine hydrochloride, megestrol acetate, trametinib, mercaptopurine, temozolomide, mitoxantrone hydrochloride, plerixafor, busulfan, azacitidine, gemtuzumab ozogamicin, vinorelbine tartrate, necitumumab, nelarabine, sorafenib tosylate, nilotinib, ixazomib citrate, nivolumab, romiplostim, obinutuzumab, ofatumumab, olaparib, omacetaxine mepesuccinate, pegaspargase, ondansetron hydrochloride, osimertinib, panitumumab, panobinostat, peginterferon alfa-2b, pembrolizumab, pertuzumab, plerixafor, pomalidomide, ponatinib hydrochloride, necitumumab, pralatrexate, procarbazine hydrochloride, aldesleukin, denosumab, ramucirumab, rasburicase, regorafenib, lenalidomide, rituximab, rolapitant hydrochloride, romidepsin, ruxolitinib phosphate, siltuximab, dasatinib, sunitinib malate, thalidomide, dabrafenib, osimertinib, talimogene, atezolizumab, temsirolimus, thalidomide, dexrazoxane hydrochloride, trabectedin, trametinib, trastuzumab, lapatinib ditosylate, dinutuximab, vandetanib, rolapitant hydrochloride, bortezomib, venetoclax, crizotinib, enzalutamide, ipilimumab, trabectedin, ziv-aflibercept, idelalisib, ceritinib, and pharmaceutically acceptable salts thereof.

### EXAMPLES

The following examples are for the purpose of illustration of the invention only and are not intended to limit the scope of the present invention in any manner whatsoever.

### Example 1. N-(4-(piperazin-1-yl)phenyl)-4-(2-(pyrrolidin-1-yl)quinazolin-6-yl)pyrimidin-2-amine

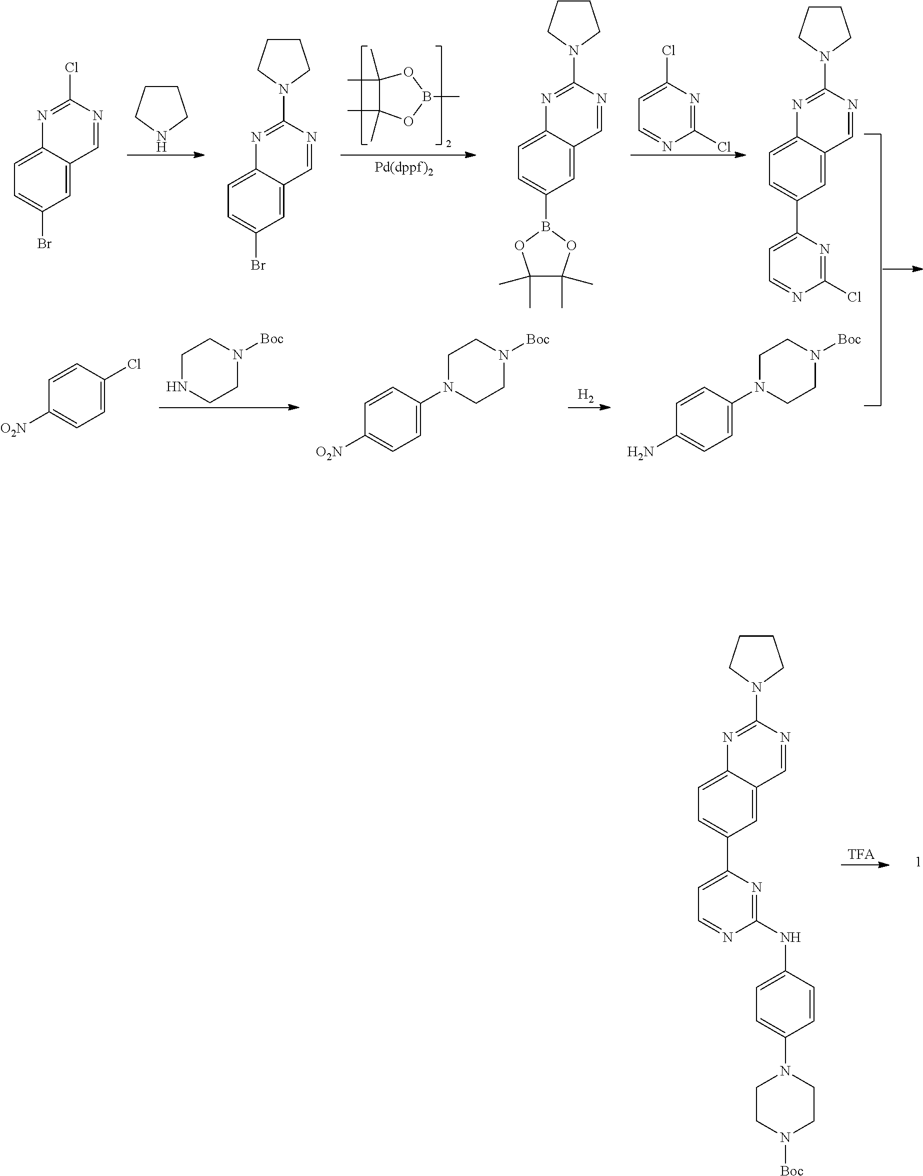

Analytical LC showed a single peak by UV absorption at 254 nM with a purity of >95%. Chemical Formula: C₂₆H₂₈N₈, Mass Spectroscopy, [M+H]⁺, calculated: 453, observed: 453.

The following compounds were prepared under analogous conditions. Each of the compounds was characterized through a combination of NHR, LC/MS, and other techniques.

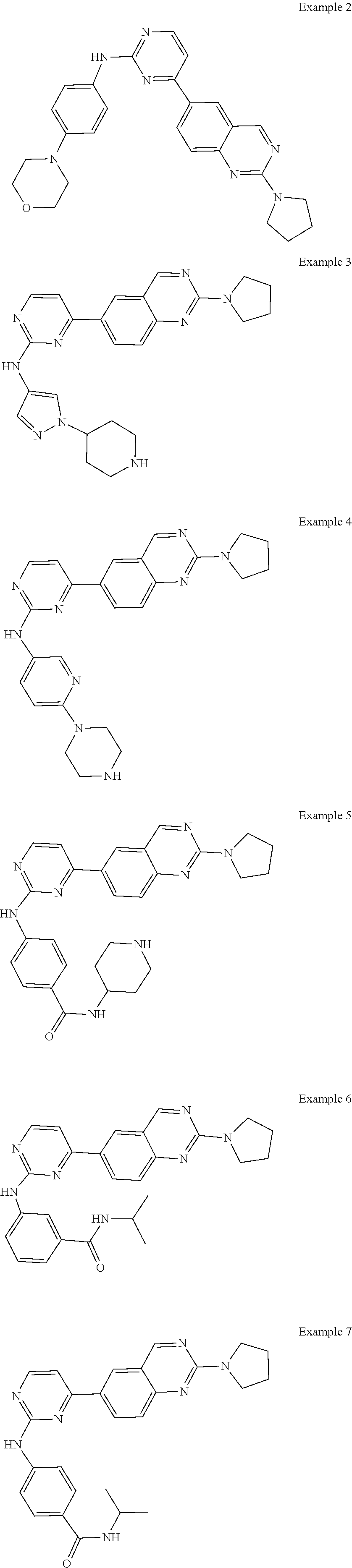 [SMILES: c1cc(-c2ccc3nc(N4CCCC4)ncc3c2)nc(Nc2ccc(N3CCOCC3)cc2)n1 ; c1cc(-c2ccc3nc(N4CCCC4)ncc3c2)nc(Nc2cnn(C3CCNCC3)c2)n1 ; c1cc(-c2ccc3nc(N4CCCC4)ncc3c2)nc(Nc2ccc(N3CCNCC3)nc2)n1 ; O=C(NC1CCNCC1)c1ccc(Nc2nccc(-c3ccc4nc(N5CCCC5)ncc4c3)n2)cc1 ; CC(C)NC(=O)c1cccc(Nc2nccc(-c3ccc4nc(N5CCCC5)ncc4c3)n2)c1 ; CC(C)NC(=O)c1ccc(Nc2nccc(-c3ccc4nc(N5CCCC5)ncc4c3)n2)cc1]

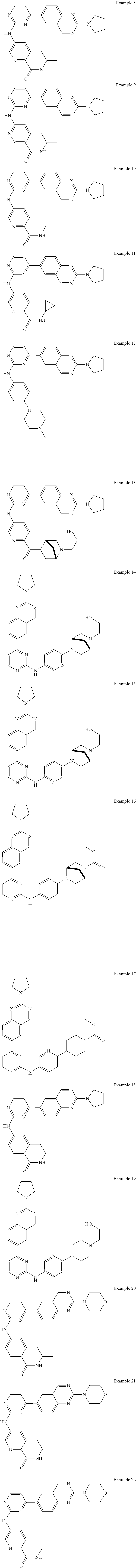

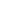

[chemical structure]

[chemical structure]

[chemical structure]

[chemical structure]

### Example 88: IC₅₀ Determinations of Enzymatic Activities of Kinase Inhibitors

All enzymatic assays and panel profiling studies are activity assays, which were carried out by Reaction Biology Corporation according to the company's protocols. IC₅₀ data against some kinases for a few selected compounds are shown in Table 1 below:

| Cpd | STK17A | MYLK4 | DDR1 |
|---|---|---|---|
| 1 | ** | *** | nd |
| 2 | *** | *** | nd |
| 3 | *** | *** | ** |
| 4 | *** | *** | nd |
| 5 | * | nd | nd |
| 6 | * | nd | nd |
| 7 | *** | ** | nd |
| 20 | *** | ** | * |
| 26 | *** | *** | nd |
| 27 | *** | *** | *** |
| 28 | *** | *** | nd |
| 29 | *** | nd | *** |
| 37 | * | * | nd |

*** IC₅₀ < 50 nM;
** 50 nM < IC₅₀ < 100 nM;
* IC₅₀ > 100 nM

### Example 89. IC₅₀ Values for Example 7 in Hematological Malignancies

| Cell type | Cell line | IC₅₀ (μM) |
|---|---|---|
| Chronic myeloid leukemia | K562 SF3B1 K666N | 0.1819 |
| | K562 | 4.789 |
| Acute lymphoblastic leukemia | NALM-6 SF3B1 K666N | 0.9078 |
| | NALM-6 | 5.505 |
| | RS4; 11 | 8.368 |
| | MOLT4 | 16.12 |
| | Jurkat | 25.72 |
| Myelodysplastic syndrome | MDSL | >100 |
| Acute myeloid leukemia | HEL92.1.7 | 0.7689 |
| | MOLM-13 | 0.9238 |
| | HEL | 3.595 |
| | MV-4-11 | 3.686 |
| | SET-2 | 3.711 |
| | Kasumi-1 | 8.1 |
| | SKNO-1 | 16.93 |
| | THP-1 | 20.04 |
| | TF1 | >100 |
| | OCI-AML3 | >100 |
| | HL-60 | >100 |
| Multiple myeloma | KMS12BM | 2.468 |
| | RPMI8226 | 8.965 |
| | OPM2 | 23.12 |
| | LP1 | 31.63 |
| | U266B1 | >100 |
| | SKMM2 | >100 |
| | KMS12PE | >100 |
| | MM-M1 | >100 |
| Lymphoma | BJAB | >100 |
| | MEC-1 | >100 |
| | RIVA | >100 |
| Non-hematological cell line | HEK293T | >100 |

### Example 90: IC₅₀ Values for Example 27 in Hematological Malignancies

| Type of cell line | Cell line | IC50 (μM) |
|---|---|---|
| Chronic myeloid leukemia | K562 | 8.713 |
| Acute lymphoblastic | Jurkat | 6.137 |
| leukemia | RS411 | 10.09 |
| | MOLT-4 | >100 |
| Acute myeloid leukemia | MOLM-13 | 0.196 |
| | MV-4-11 | 0.3213 |
| | Kasumi-1 | 0.5301 |
| | SET-2 | 0.6484 |
| | OCI-AML3 | 0.8862 |
| | THP-1 | 1.152 |
| | HL60 | 5.00 |
| | SKNO-1 | 14.84 |
| | TF1 | 15.00 |
| | HEL | >100 |
| Multiple myeloma | LP1 | 1.107 |
| | RPMI8226 | 2.697 |
| | KMS12BM | 2.808 |
| | KMS12PE | 2.814 |
| | SKMM2 | 6.623 |
| | OPM2 | 6.854 |
| | MM-M1 | 8.073 |
| | U266B1 | 12.58 |
| Lymphoma | RIVA | 9.201 |
| | BJAB | 12.07 |
| | MEC-1 | 14.21 |
| Non-hematological cell line | HEK293T | >100 |

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1-132. (canceled)

133. A compound having the formula: 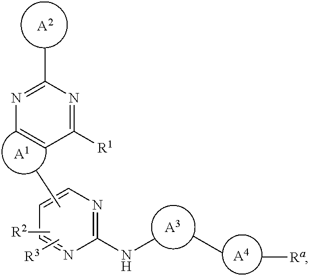
or a pharmaceutically acceptable salt thereof, wherein
A¹ is aryl, cycloalkyl, heteroaryl, or heterocyclyl group
A² is an alkyl, cycloalkyl, heteroaryl or heterocyclyl group, or Q¹-NR^{6a}R^{6b}, wherein Q¹ is null or C₁₋₄alkylene, R^{6a} is hydrogen, C₁₋₈alkyl, aryl, C₁₋₈heteroaryl, C₃₋₈cycloalkyl, or C₁₋₈heterocyclyl, R^{6b} is hydrogen, C₁₋₈alkyl, aryl, C₁₋₈heteroaryl, C₃₋₈cycloalkyl, or C₁₋₈ heterocyclyl;
A³ is null or an aryl, cycloalkyl, heteroaryl, or heterocyclyl group
A⁴ is null or an aryl, cycloalkyl, heteroaryl, or heterocyclyl group
R¹ is hydrogen, C₁₋₈alkyl, or C₃₋₈cycloalkyl;
R² is F, Cl, Br, I, NO₂, CN, R^{2*}, OR^{2*}, N(R^{2*})₂, SO₃R^{2*}, SO₂R^{2*}, SO₂N(R^{2*})₂, C(O)R^{2*}; C(O)OR², OC(O)R^{2*}; C(O)N(R^{2*})₂, N(R^{2*})C(O)R^{2*}, OC(O)N(R^{2*})₂, N(R^{2*})C(O)N(R^{2*})₂, wherein R^{2*} is in each case independently selected from hydrogen, C₁₋₈alkyl, aryl, C₁₋₈heteroaryl, C₃₋₈cycloalkyl, or C₁₋₈heterocyclyl;
R³ is F, Cl, Br, I, NO₂, CN, R^{3*}, OR^{3*}, N(R^{3*})₂, SO₃R^{3*}, SO₂R^{3*}, SO₂N(R^{3*})₂, C(O)R^{3*}; C(O)OR^{3*}, OC(O)R^{3*}; C(O)N(R^{3*})₂, N(R^{3*})C(O)R^{3*}, OC(O)N(R^{3*})₂, N(R^{3*})C(O)N(R^{3*})₂, wherein R^{3*} is in each case independently selected from hydrogen, C₁₋₈alkyl, aryl, C₁₋₈heteroaryl, C₃₋₈cycloalkyl, or C₁₋₈heterocyclyl;
R^{a} has the formula:
—Z³—Z^{c}—Z⁴—Z^{t},
wherein
Z³ is null, C₁₋₆alkylene, C₃₋₈cycloalkylene, arylene, C₁₋₈hetercyclylene, or C₁₋₈heteroarylene,
Z^{c} is -null, C(═O)—, SO₂, —C(═O)O—, —C(═O)NH—
Z⁴ is null, C₁₋₆alkylene, C₃₋₈cycloalkylene, arylene, C₁₋₈hetercyclylene, or C₁₋₈heteroarylene,
Z^{t} represents a bond to A⁴ or A³, or is F, Cl, Br, I, CN, NO₂, R^{za*}, OR^{za*}, SR^{za*}, N(R^{za*})₂, C(═O)R^{za*}, C(═O)OR^{za*}, C(═O)NHR^{za*},
wherein R^{za*} is in each case independently selected from hydrogen, C₁₋₈alkyl, aryl, C₁₋₈ heteroaryl, C₃₋₈cycloalkyl, or C₁₋₈heterocyclyl;
wherein any two of more A¹, A², A³, A⁴, R¹, R², R³, and R^{a} may together form a ring.

134. The compound according to claim 133, having the formula: 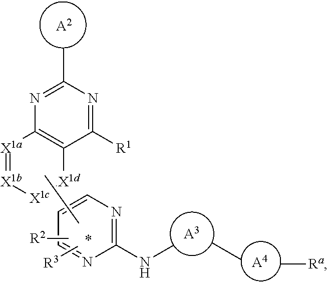
wherein
X^{1a} is selected from N and CR;
X^{2a} is selected from N and CR;
X^{3a} is selected from N and CR;
X^{4a} is selected from N and CR;
wherein one R group is a single bond to the *-pyrimidine, and the remaining R groups are independently selected from F, Cl, Br, I, NO₂, CN, R*, OR*, N(R*)₂, SO₃R*, SO₂R*, SO₂N(R*)₂, C(O)R*; C(O)OR*, OC(O)R*; C(O)N(R*)₂, N(R*)C(O)R*, OC(O)N(R*)₂, N(R*)C(O)N(R*)₂, wherein R* is in each case independently selected from hydrogen, C₁₋₈alkyl, aryl, C₁₋₈heteroaryl, C₃₋₈cycloalkyl, or C₁₋₈heterocyclyl.
wherein any two of more A¹, A², A³, A⁴, R¹, R², R³, R, and R^{a} may together form a ring.

135. The compound according to claim 134, having the formula: 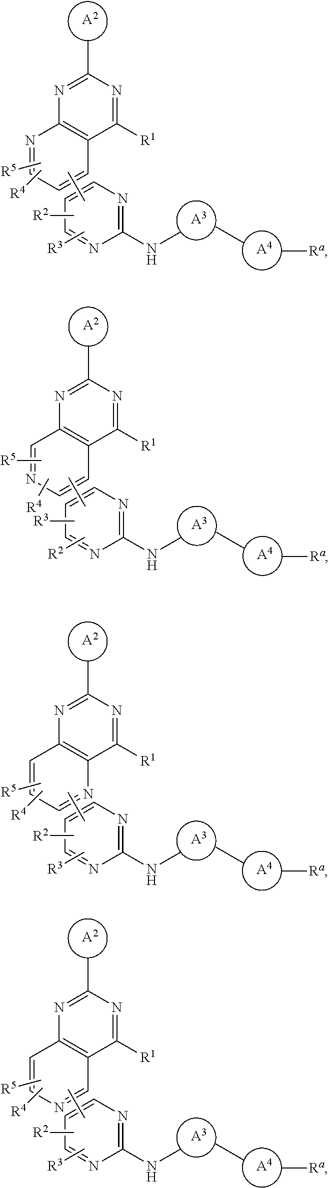 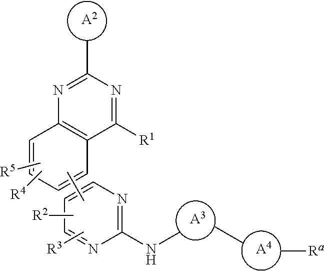
wherein
R⁴ is F, Cl, Br, I, NO₂, CN, R^{4*}, OR^{4*}, N(R^{4*})₂, SO₃R^{4*}, SO₂R^{4*}, SO₂N(R^{4*})₂, C(O)R^{4*}; C(O)OR^{4*}, OC(O)R^{4*}; C(O)N(R^{4*})₂, N(R^{4*})C(O)R^{4*}, OC(O)N(R^{4*})₂, N(R^{4*})C(O)N(R^{4*})₂, wherein R^{4*} is in each case independently selected from hydrogen, C₁₋₈alkyl, aryl, C₁₋₈heteroaryl, C₃₋₈cycloalkyl, or C₁₋₈heterocyclyl;
R⁵ is F, Cl, Br, I, NO₂, CN, R^{5*}, OR^{5*}, N(R^{5*})₂, SO₃R^{5*}, SO₂R^{5*}, SO₂N(R^{5*})₂, C(O)R^{5*}; C(O)OR^{5*}, OC(O)R^{5*}; C(O)N(R^{5*})₂, N(R^{5*})C(O)R^{5*}, OC(O)N(R^{5*})₂, N(R^{5*})C(O)N(R^{5*})₂, wherein R^{5*} is in each case independently selected from hydrogen, C₁₋₈alkyl, aryl, C₁₋₈heteroaryl, C₃₋₈cycloalkyl, or C₁₋₈heterocyclyl;
wherein any two of more of A², A³, A⁴, R¹, R², R³, R⁴, R⁵, and R^{a} may together form a ring.

136. The compound according to claim 135, having the formula:

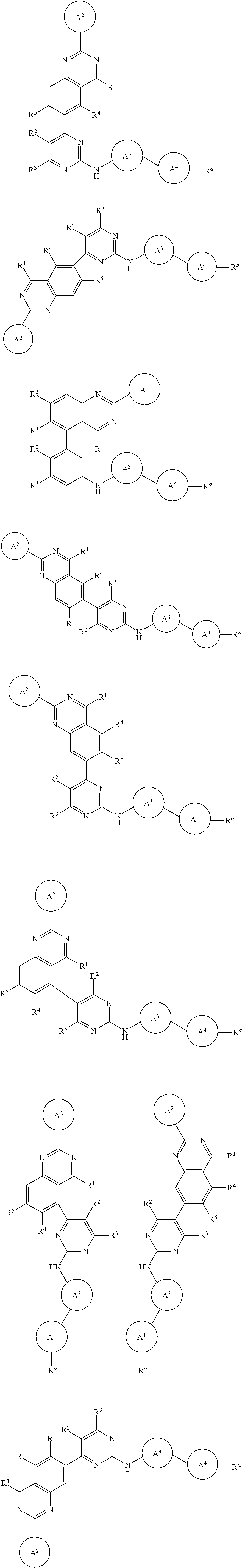

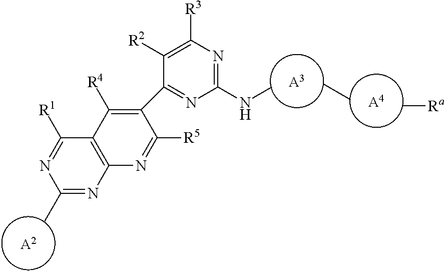

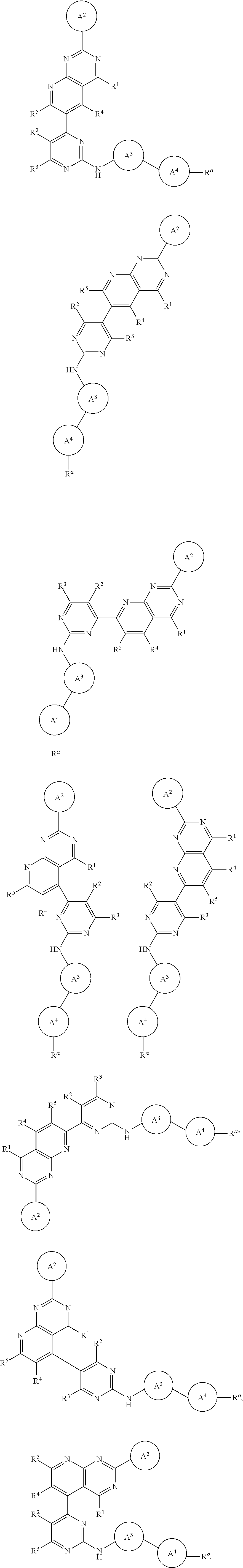

137. The compound according to claim 135, having the formula: 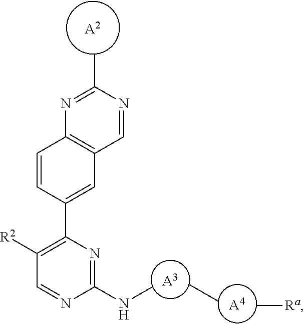
wherein R² is H or F

138. The compound according to claim 135, wherein A² has the formula: 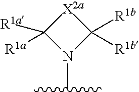
wherein X^{a2} is selected from Z, Z—O, Z—S, Z—NRⁿ¹,
wherein Z is null or a group having the formula: 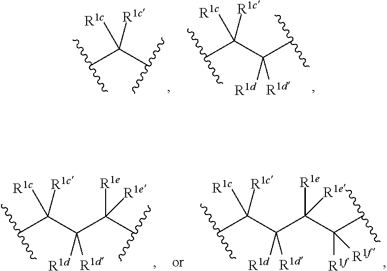
wherein Rⁿ¹ is selected from is hydrogen or C₁₋₈alkyl;
R^{1a} is selected from F, Cl, Br, I, NO₂, CN, R^{1a*}, OR^{1a*}, N(R^{1a*})₂, SO₃R^{1a*}, SO₂R^{1a*}, SO₂N(R^{1a*})₂, C(O)R^{1a*}; C(O)OR^{1a*}, OC(O)R^{1a*}; C(O)N(R^{1a*})₂, N(R^{1a*})C(O)R^{1a*}, OC(O)N(R^{1a*})₂, N(R^{1a*})C(O)N(R^{1a*})₂, wherein R^{1a*} is in each case independently selected from hydrogen, C₁₋₈alkyl, aryl, C₁₋₈heteroaryl, C₃₋₈cycloalkyl, or C₁₋₈heterocyclyl;
R^{1a′} is selected from F, Cl, Br, I, NO₂, CN, R^{1a′*}, OR^{1a′*}, N(R^{1a′*})₂, SO₃R^{1a′*}, SO₂R^{1a′*}, SO₂N(R^{1a′*})₂, C(O)R^{1a′*}; C(O)O R^{1a′*}, OC(O)R^{1a′*}; C(O)N(R^{1a′*})₂, N(R^{1a′*}) C(O)R^{1a′*}, OC(O)N(R^{1a′*})₂, N(R^{1a′*})C(O)N(R^{1a′*})₂, wherein R^{1a′*} is in each case independently selected from hydrogen, C₁₋₈alkyl, aryl, C₁₋₈heteroaryl, C₃₋₈cycloalkyl, or C₁₋₈heterocyclyl; or
wherein R^{1a} and R^{1a′} together form an oxo, or thiooxo;
R^{1b} is selected from F, Cl, Br, I, NO₂, CN, R^{1b*}, OR^{1b*}, N(R^{1b*})₂, SO₃R^{1b*}, SO₂R^{1b*}, SO₂N(R^{1b*})₂, C(O)R^{1b*}; C(O)OR^{1b*}, OC(O)R^{1b*}; C(O)N(R^{1b*})₂, N(R^{1b*})C(O)R^{1b*}, OC(O)N(R^{1b*})₂, N(R^{1b*})C(O)N(R^{1b*})₂, wherein R^{1b*} is in each case independently selected from hydrogen, C₁₋₈alkyl, aryl, C₁₋₈heteroaryl, C₃₋₈cycloalkyl, or C₁₋₈heterocyclyl;
R^{1b′} is selected from F, Cl, Br, I, NO₂, CN, R^{1b′*}, OR^{1b′*}, N(R^{1b′*})₂, SO₃R^{1b′*}, SO₂R^{1b′*}, SO₂N(R^{1b′*})₂, C(O)R^{1b′*}; C(O)OR^{1b′*}, OC(O)R^{1b′*}; C(O)N(R^{1b′*})₂, N(R^{1b′*})C(O)R^{1b′*}, OC(O)N(R^{1b′*})₂, N(R^{1b′*})C(O)N(R^{1b′*})₂, wherein R^{1b′*} is in each case independently selected from hydrogen, C₁₋₈alkyl, aryl, C₁₋₈heteroaryl, C₃₋₈cycloalkyl, or C₁₋₈heterocyclyl; or
wherein R^{1b} and R^{1b′} together form an oxo, or thiooxo
R^{1c} is selected from F, Cl, Br, I, NO₂, CN, R^{1c*}, OR^{1c*}, N(R^{1c*})₂, SO₃R^{1c*}, SO₂R^{1c*}, SO₂N(R^{1c*})₂, C(O)R^{1c*}; C(O)OR^{1c*}, OC(O)R^{1c*}; C(O)N(R^{1c*})₂, N(R^{1c*})C(O)R^{1c*}, OC(O)N(R^{1c*})₂, N(R^{1c*})C(O)N(R^{1c*})₂, wherein R^{1c*} is in each case independently selected from hydrogen, C₁₋₈alkyl, aryl, C₁₋₈heteroaryl, C₃₋₈cycloalkyl, or C₁₋₈heterocyclyl;
R^{1c′} is selected from F, Cl, Br, I, NO₂, CN, R^{1c′*}, OR^{1c′*}, N(R^{1c′*})₂, SO₃R^{1c′*}, SO₂R^{1c′*}, SO₂N(R^{1c′*})₂, C(O)R^{1c′*}; C(O)OR^{1c′*}, OC(O)R^{1c′*}; C(O)N(R^{1c′*})₂, N(R^{1c′*})C(O)R^{1c′*}, OC(O)N(R^{1c′*})₂, N(R^{1c′*})C(O)N(R^{1c′*})₂, wherein R^{1c′*} is in each case independently selected from hydrogen, C₁₋₈alkyl, aryl, C₁₋₈heteroaryl, C₃₋₈cycloalkyl, or C₁₋₈heterocyclyl; or
wherein R^{1c} and R^{1c′} together form an oxo, or thiooxo
R^{1d} is selected from F, Cl, Br, I, NO₂, CN, R^{1d*}, OR^{1d*}, N(R^{1d*})₂, SO₃R^{1d*}, SO₂R^{1d*}, SO₂N(R^{1d*})₂, C(O)R^{1d*}; C(O)OR^{1d*}, OC(O)R^{1d*}; C(O)N(R^{1d*})₂, N(R^{1d*})C(O)R^{1d*}, OC(O)N(R^{1d*})₂, N(R^{1d*})C(O)N(R^{1d*})₂, wherein R^{1d*} is in each case independently selected from hydrogen, C₁₋₈alkyl, aryl, C₁₋₈heteroaryl, C₃₋₈cycloalkyl, or C₁₋₈heterocyclyl;
R^{1d′} is selected from F, Cl, Br, I, NO₂, CN, R^{1d′*}, OR^{1d′*}, N(R^{1d′*})₂, SO₃R^{1d′*}, SO₂R^{1d′*}, SO₂N(R^{1d′*})₂, C(O)R^{1d′*}; C(O)OR^{1d′*}, OC(O)R^{1d′*}; C(O)N(R^{1d′*})₂, N(R^{1d′*})C(O)R^{1d′*}, OC(O)N(R^{1d′*})₂, N(R^{1d′*})C(O)N(R^{1d′*})₂, wherein R^{1d′*} is in each case independently selected from hydrogen, C₁₋₈alkyl, aryl, C₁₋₈heteroaryl, C₃₋₈cycloalkyl, or C₁₋₈heterocyclyl; or
wherein R^{1d} and R^{1d′} together form an oxo, or thiooxo
R^{1e} is selected from F, Cl, Br, I, NO₂, CN, R^{1e*}, OR^{1e*}, N(R^{1e*})₂, SO₃R^{1e*}, SO₂R^{1e*}, SO₂N(R^{1e*})₂, C(O)R^{1e*}; C(O)OR^{1e*}, OC(O)R^{1e*}; C(O)N(R^{1e*})₂, N(R^{1e*})C(O)R^{1e*}, OC(O)N(R^{1e*})₂, N(R^{1e*})C(O)N(R^{1e*})₂, wherein R^{1e*} is in each case independently selected from hydrogen, C₁₋₈alkyl, aryl, C₁₋₈heteroaryl, C₃₋₈cycloalkyl, or C₁₋₈heterocyclyl;
R^{1e′} is selected from F, Cl, Br, I, NO₂, CN, R^{1e′*}, OR^{1e′*}, N(R^{1e′*})₂, SO₃R^{1e′*}, SO₂R^{1e′*}, SO₂N(R^{1e′*})₂, C(O)R^{1e′*}; C(O)OR^{1e′*}, OC(O)R^{1e′*}; C(O)N(R^{1e′*})₂, N(R^{1e′*})C(O)R^{1e′*}, OC(O)N(R^{1e′*})₂, N(R^{1e′*})C(O)N(R^{1e′*})₂, wherein R^{1e′*} is in each case independently selected from hydrogen, C₁₋₈alkyl, aryl, C₁₋₈heteroaryl, C₃₋₈cycloalkyl, or C₁₋₈heterocyclyl; or
wherein R^{1e} and R^{1e′} together form an oxo, or thiooxo
R^{1f} is selected from F, Cl, Br, I, NO₂, CN, R^{1f*}, OR^{1f*}, N(R^{1f*})₂, SO₃R^{1f*}, SO₂R^{1f*}, SO₂N(R^{1f*})₂, C(O)R^{1f*}; C(O)OR^{1f*}, OC(O)R^{1f*}; C(O)N(R^{1f*})₂, N(R^{1f*})C(O)R^{1f*}, OC(O)N(R^{1f*})₂, N(R^{1f*})C(O)N(R^{1f*})₂, wherein R^{1f*} is in each case independently selected from hydrogen, C₁₋₈alkyl, aryl, C₁₋₈heteroaryl, C₃₋₈cycloalkyl, or C₁₋₈heterocyclyl;
R^{1f′} is selected from F, Cl, Br, I, NO₂, CN, R^{1f′*}, OR^{1f′*}, N(R^{1f′*})₂, SO₃R^{1f′*}, SO₂R^{1f′*}, SO₂N(R^{1f′*})₂, C(O)R^{1f′*}; C(O)OR^{1f′}*, OC(O)R^{1f′*}; C(O)N(R^{1f′*})₂, N(R^{1f′*})C(O)R^{1f′*}, OC(O)N(R^{1f′*})₂, N(R^{1f′*})C(O)N(R^{1f′*})₂, wherein R^{1f′*} is in each case independently selected from hydrogen, C₁₋₈alkyl, aryl, C₁₋₈heteroaryl, C₃₋₈cycloalkyl, or C₁₋₈heterocyclyl; or
wherein R^{1f} and R^{1f′} together form an oxo, or thiooxo;
wherein any two adjacent R groups may together form a double bond;
wherein any two or more of R^{1a}, R^{1a′}, R^{1b}, R^{1b′}, R^{1c}, R^{11c′}, R^{1d}, R^{1d′}, R^{1e}, R^{1e′}, R^{1f}, R^{1f′}, and Rⁿ¹ may together form a ring

139. The compound according to claim 138, wherein A² has the formula: 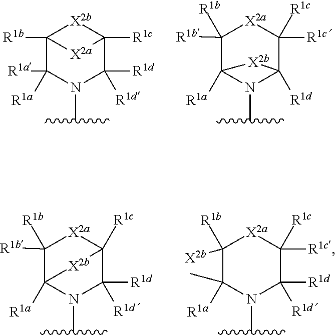
wherein X^{2b} is selected from Z¹, Z¹—O—Z¹, Z¹—S—Z¹, Z¹—NRⁿ²—Z¹, wherein Z¹ is null or a group having the formula: 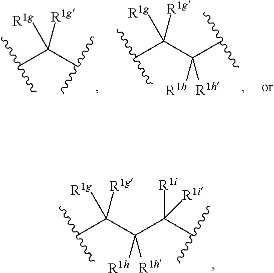
wherein Rⁿ² is selected from H or C₁₋₈alkyl,
wherein R^{1g} is selected from F, Cl, Br, I, NO₂, CN, R^{1g*}, OR^{1g*} N(R^{1g*})₂, SO₃R^{1g*}, SO₂R^{1g*}, SO₂N(R^{1g*})₂, C(O)R^{1g*}; C(O)OR^{1g*}, OC(O)R^{1g*}; C(O)N(R^{1g*})₂, N(R^{1g*})C(O)R^{1g*}, OC(O)N(R^{1g*})₂, N(R^{1g*})C(O)N(R^{1g*})₂, wherein R^{1g*} is in each case independently selected from hydrogen, C₁₋₈alkyl, aryl, C₁₋₈heteroaryl, C₃₋₈cycloalkyl, or C₁₋₈heterocyclyl;
wherein R^{1g′} is selected from F, Cl, Br, I, NO₂, CN, R^{1g′*}, OR^{1g′*}, N(R^{1g′*})₂, SO₃R^{1g′*}, SO₂R^{1g′*}, SO₂N(R^{1g′*})₂, C(O)R^{1g′*}; C(O)O R^{1g′*}, OC(O)R^{1g′*}; C(O)N(R^{1g′*})₂, N(R^{1g′*})C(O)R^{1g′*}, OC(O)N(R^{1g′*})₂, N(R^{1g′*})C(O)N(R^{1g′*})₂, wherein R^{1g′*} is in each case independently selected from hydrogen, C₁₋₈alkyl, aryl, C₁₋₈heteroaryl, C₃₋₈cycloalkyl, or C₁₋₈heterocyclyl; or
wherein R^{1g} and R^{1g′} together form an oxo, or thiooxo
wherein R^{1h} is selected from F, Cl, Br, I, NO₂, CN, R^{1h*}, OR^{1h*}, N(R^{1h*})₂, SO₃R^{1h*}, SO₂R^{1h*}, SO₂N(R^{1h*})₂, C(O)R^{1h*}; C(O)OR^{1h*}, OC(O)R^{1h*}; C(O)N(R^{1h*})₂, N(R^{1h*})C(O)R^{1h*} OC(O)N(R^{1h*})₂, N(R^{1h*})C(O)N(R^{1h*})₂, wherein R^{1h*} is in each case independently selected from hydrogen, C₁₋₈alkyl, aryl, C₁₋₈heteroaryl, C₃₋₈cycloalkyl, or C₁₋₈ heterocyclyl;
wherein R^{1h′} is selected from F, Cl, Br, I, NO₂, CN, R^{1h′*}, OR^{1h′*}, N(R^{1h′*})₂, SO₃R^{1h′*}, SO₂R^{1h′*}, SO₂N(R^{1h′*})₂, C(O)R^{1h′*}; C(O)OR^{1h′*}, OC(O)R^{1h′*}; C(O)N(R^{1h′*})₂, N(R^{1h′*})C(O)R^{1h′*}, OC(O)N(R^{1h′*})₂, N(R^{1h′*})C(O)N(R^{1h′*})₂, wherein R^{1h′*} is in each case independently selected from hydrogen, C₁₋₈alkyl, aryl, C₁₋₈heteroaryl, C₃₋₈cycloalkyl, or C₁₋₈ heterocyclyl; or
wherein R^{1h} and R^{1h′} together form an oxo, or thiooxo
wherein R¹ⁱ is selected from F, Cl, Br, I, NO₂, CN, R^{1i*}, OR^{1i*}, N(R^{1i*})₂, SO₃R^{1i*}, SO₂R^{1i*}, SO₂N(R^{1i*})₂, C(O)R^{1i*}; C(O)OR^{1i*}, OC(O)R^{1i*}; C(O)N(R^{1i*})₂, N(R^{1i*})C(O)R^{1i*}, OC(O)N(R^{1i*})₂, N(R^{1i*})C(O)N(R^{1i*})₂, wherein R^{1i*} is in each case independently selected from hydrogen, C₁₋₈alkyl, aryl, C₁₋₈heteroaryl, C₃₋₈cycloalkyl, or C₁₋₈heterocyclyl;
wherein R^{1i′} is selected from F, Cl, Br, I, NO₂, CN, R^{1i′*}, OR^{1i′*}, N(R^{1i′*})₂, SO₃R^{1i′*}, SO₂R^{1i′*}, SO₂N(R^{1i′*})₂, C(O)R^{1i′*}; C(O)OR^{1i′*}, OC(O)R^{1i′*}; C(O)N(R^{1i′*})₂, N(R^{1i′*})C(O)R^{1i′*}, OC(O)N(R^{1i′*})₂, N(R^{1i′*})C(O)N(R^{1i′*})₂, wherein R^{1i′*} is in each case independently selected from hydrogen, C₁₋₈alkyl, aryl, C₁₋₈heteroaryl, C₃₋₈cycloalkyl, or C₁₋₈ heterocyclyl; or
wherein R¹ⁱ and R^{1i′} together form an oxo, or thiooxo
wherein any two adjacent R groups may together form a double bond;
wherein any two or more of R^{1a}, R^{1a′}, R^{1b}, R^{1b′}, R^{1c}, R^{1c′}, R^{1d}, R^{1d′}, R^{1e}, R^{1e′}, R^{1f}, R^{1f′}, R^{1g}, R^{1g′}, R^{1g}, R^{1g′}, R¹ⁱ, and R¹ⁱ may together form a ring.

140. The compound according to claim 138, wherein A² has the formula: 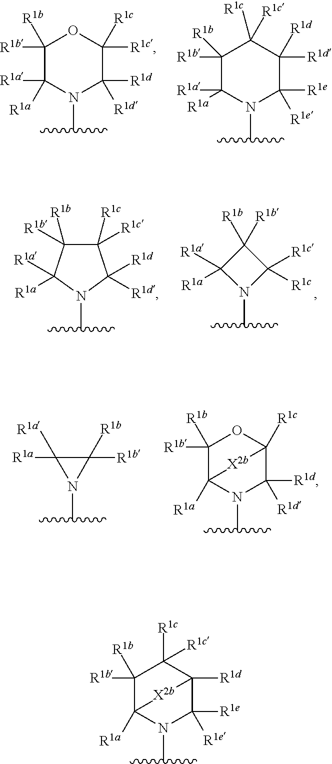
wherein X^{2b} is:

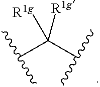

141. The compound according to claim 138, wherein A² has the formula:

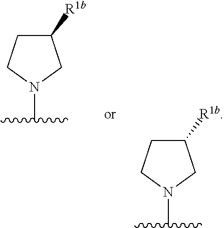

142. The compound according to claim 133, wherein A³ has the formula: 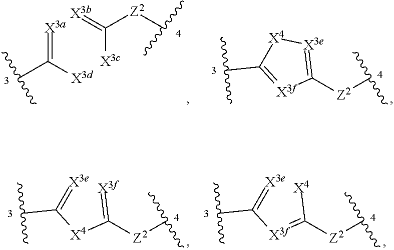
wherein
X^{3a} is CR³a or N,
X^{3b} is CR^{3b} or N,
X^{3c} is CR^{3c} or N,
X^{3d} is CR^{3d} or N,
X^{3e} is CR^{3e} or N,
X^{3f} is CR^{3f} or N,
X⁴ is NRⁿ³, S, O,
Z² is null, O, S, NRⁿ⁴;
R^{3a} is F, Cl, Br, I, NO₂, CN, R^{3a*}, OR^{3*}, N(R^{3a*})₂, SO₃R^{3a*}, SO₂R^{3a*}, SO₂N(R^{3a*})₂, C(O)R^{3a*}; C(O)OR^{3a*}, OC(O)R^{3a*}; C(O)N(R^{3a*})₂, N(R^{3a*})C(O)R^{3a*}, OC(O)N(R^{3a*})₂, N(R^{3a*})C(O)N(R^{3a*})₂, wherein R^{3a*} is in each case independently selected from hydrogen, C₁₋₈alkyl, aryl, C₁₋₈heteroaryl, C₃₋₈cycloalkyl, or C₁₋₈heterocyclyl;
R^{3b} is F, Cl, Br, I, NO₂, CN, R^{3b*}, OR^{3b*}, N(R^{3b*})₂, SO₃R^{3b*}, SO₂R^{3b*}, SO₂N(R^{3b*})₂, C(O)R^{3b*}; C(O)OR^{3b*}, OC(O)R³bb*; C(O)N(R^{3b*})₂, N(R^{3b*})C(O)R³bb*, OC(O)N(R^{3b*})₂, N(R^{3b*})C(O)N(R^{3b*})₂, wherein R^{3b*} is in each case independently selected from hydrogen, C₁₋₈alkyl, aryl, C₁₋₈heteroaryl, C₃₋₈cycloalkyl, or C₁₋₈heterocyclyl;
R^{3c} is F, Cl, Br, I, NO₂, CN, R^{3c*}, OR^{3c*}, N(R^{3c*})₂, SO₃R^{3c*}, SO₂R^{3c*}, SO₂N(R^{3c*})₂, C(O)R^{3c*}; C(O)OR^{3c*}, OC(O)R^{3c*}; C(O)N(R^{3c*})₂, N(R^{3c*})C(O)R^{3c*}, OC(O)N(R^{3c*})₂, N(R^{3c*})C(O)N(R^{3c*})₂, wherein R^{3c*} is in each case independently selected from hydrogen, C₁₋₈alkyl, aryl, C₁₋₈heteroaryl, C₃₋₈cycloalkyl, or C₁₋₈heterocyclyl;
R^{3d} is F, Cl, Br, I, NO₂, CN, R^{3d*}, OR^{3d*}, N(R^{3d*})₂, SO₃R^{3d*}, SO₂R^{3d*}, SO₂N(R^{3d*})₂, C(O)R^{3d*}; C(O)OR^{3d*}, OC(O)R³dd*; C(O)N(R^{3d*})₂, N(R^{3d*})C(O)R^{3d*}, OC(O)N(R^{3d*})₂, N(R^{3d*})C(O)N(R^{3d*})₂, wherein R^{3d*} is in each case independently selected from hydrogen, C₁₋₈alkyl, aryl, C₁₋₈heteroaryl, C₃₋₈cycloalkyl, or C₁₋₈heterocyclyl;
R^{3e} is F, Cl, Br, I, NO₂, CN, R^{3e*}, OR^{3e*}, N(R^{3e*})₂, SO₃R^{3e*}, SO₂R^{3e*}, SO₂N(R^{3e*})₂, C(O)R^{3e*}; C(O)OR^{3e*}, OC(O)R^{3e*}; C(O)N(R^{3e*})₂, N(R^{3e*})C(O)R^{3e*}, OC(O)N(R^{3e*})₂, N(R^{3e*})C(O)N(R^{3e*})₂, wherein R^{3e*} is in each case independently selected from hydrogen, C₁₋₈alkyl, aryl, C₁₋₈heteroaryl, C₃₋₈cycloalkyl, or C₁₋₈heterocyclyl;
R^{3f} is F, Cl, Br, I, NO₂, CN, R^{3f*}, OR^{3f*}, N(R^{3f*})₂, SO₃R^{3f*}, SO₂R^{3f*}, SO₂N(R^{3f*})₂, C(O)R^{3f*}; C(O)OR^{3f*}, OC(O)R^{3f*}; C(O)N(R^{3f*})₂, N(R^{3f*})C(O)R^{3f*}, OC(O)N(R^{3f*})₂, N(R^{3f*})C(O)N(R^{3f*})₂, wherein R^{3f*} is in each case independently selected from hydrogen, C₁₋₈alkyl, aryl, C₁₋₈heteroaryl, C₃₋₈cycloalkyl, or C₁₋₈heterocyclyl;
R^{g} is F, Cl, Br, I, NO₂, CN, R^{3g*}, OR^{3g*}, N(R^{3g*})₂, SO₃R^{3g*}, SO₂R^{3g*}, SO₂N(R^{3g*})₂, C(O)R^{3g*}; C(O)OR^{3g*}, OC(O)R^{3g*}; C(O)N(R^{3g*})₂, N(R^{3g*})C(O)R^{3g*}, OC(O)N(R^{3g*})₂, N(R^{3g*})C(O)N(R^{3g*})₂, wherein R^{3g*} is in each case independently selected from hydrogen, C₁₋₈alkyl, aryl, C₁₋₈heteroaryl, C₃₋₈cycloalkyl, or C₁₋₈heterocyclyl;
Rⁿ³ is hydrogen, C₁₋₈alkyl, aryl, C₁₋₈heteroaryl, C₃₋₈cycloalkyl, or C₁₋₈heterocyclyl;
Rⁿ⁴ is hydrogen, C₁₋₈alkyl, aryl, C₁₋₈heteroaryl, C₃₋₈cycloalkyl, or C₁₋₈heterocyclyl;
wherein any one of R^{3a}, R^{3b}, R^{3c} R^{3d}, R^{3e}, R^{3f}, Rⁿ³, or Rⁿ⁴ may form a bond with Z^{t},
R^{3a} and R^{3b} may together form a ring;
R^{3c} and R^{3d} may together form a ring;
any two of R^{3e}, R^{3f}, and Rⁿ³ may together form a ring;
wavy line 3 represents the point of attachment to NRⁿ, and
wavy line 4 represents the point of attachment to A⁴;
wherein any two or more of R^{1a}, R^{1a′}, R^{1b}, R^{1b′}, R^{1c}, R^{1c′}, R^{1d}, R^{1d′}, R^{1e}, R^{1e′}, R^{1f}, R^{1f′}, Rⁿ¹, R^{1g}, R^{1g′}, R^{1g}, R^{1g′}, R¹ⁱ, R¹ⁱ, R^{3a}, R^{3b}, R^{3c}, R^{3d}, R^{3f}, R^{3e}, R^{3f}, and Rⁿ³ may together form a ring.

143. The compound according to claim 142, wherein A³ has the formula:

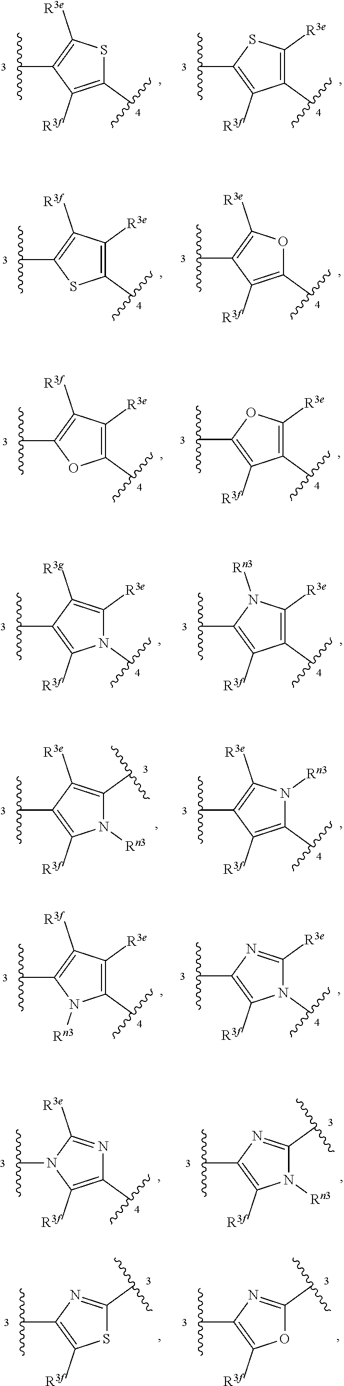

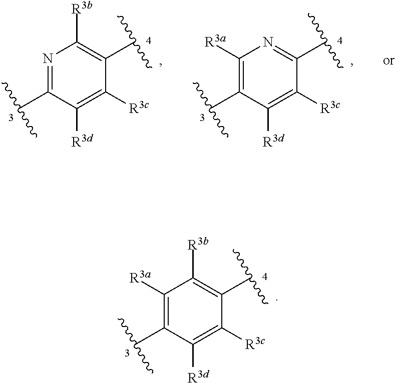

144. The compound according to claim 133, wherein the A⁴ has the formula: 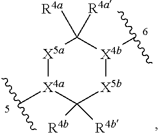
wherein
R^{4a} is F, Cl, Br, I, NO₂, CN, R^{4a*}, OR^{4a*}, N(R^{4a*})₂, SO₃R^{4a*}, SO₂R^{4a*}, SO₂N(R^{4a*})₂, C(O)R^{4a*}; C(O)OR^{4a*}, OC(O)R^{4a*}; C(O)N(R^{4a*})₂, N(R^{4a*})C(O)R^{4a*}, OC(O)N(R^{4a*})₂, N(R^{4a*})C(O)N(R^{4a*})₂, wherein R^{4a*} is in each case independently selected from hydrogen, C₁₋₈alkyl, aryl, C₁₋₈heteroaryl, C₃₋₈cycloalkyl, or C₁₋₈heterocyclyl;
R^{4a′} is F, Cl, Br, I, NO₂, CN, R^{4a′*}, OR^{4a′*}, N(R^{4a′*})₂, SO₃R^{4a′*}, SO₂R^{4a′*}, SO₂N(R^{4a′*})₂, C(O)R^{4a′*}; C(O)OR^{4a′*}, OC(O)R^{4a′*}; C(O)N(R^{4a′*})₂, N(R^{4a′*})C(O)R^{4a′*}, OC(O)N(R^{4a′*})₂, N(R^{4a′*})C(O)N(R^{4a′*})₂, wherein R^{4a′*} is in each case independently selected from hydrogen, C₁₋₈alkyl, aryl, C₁₋₈heteroaryl, C₃₋₈cycloalkyl, or C₁₋₈heterocyclyl;
wherein R^{4a} and R^{4a′} may together form an oxo or thiooxo;
R^{4b} is F, Cl, Br, I, NO₂, CN, R^{4b′*}, OR^{4b′*}, N(R^{4b′*})₂, SO₃R^{4b′*}, SO₂R^{4b′*}, SO₂N(R^{4b′*})₂, C(O)R^{4b′*}; C(O)OR^{4b}, OC(O)R^{4b*}; C(O)N(R^{4b*})₂, N(R^{4b*})C(O)R^{4b*}, OC(O)N(R^{4b*})₂, N(R^{4b′*})C(O)N(R^{4b′*})₂, wherein R^{4b′*} is in each case independently selected from hydrogen, C₁₋₈alkyl, aryl, C₁₋₈heteroaryl, C₃₋₈cycloalkyl, or C₁₋₈heterocyclyl;
R^{4b′} is F, Cl, Br, I, NO₂, CN, R^{4b′*}, OR^{4b′*}, N(R^{4b′*})₂, SO₃R^{4b′*}, SO₂R^{4b′*}, SO₂N(R^{4b′*})₂, C(O)R^{4b′*}; C(O)OR^{4b′*}, OC(O)R^{4b′*}; C(O)N(R^{4b′*})₂, N(R^{4b′*})C(O)R^{4b′*}, OC(O)N(R^{4b′*})₂, N(R^{4b′*})C(O)N(R^{4b′*})₂, wherein R^{4b′*} is in each case independently selected from hydrogen, C₁₋₈alkyl, aryl, C₁₋₈heteroaryl, C₃₋₈cycloalkyl, or C₁₋₈heterocyclyl;
wherein R^{4b} and R^{4b′} may together form an oxo or thiooxo;
X^{4a} is N or CR^{4c}
X^{4b} is N or CR^{4d}
R^{4c} is F, Cl, Br, I, NO₂, CN, R^{4c*}, OR^{4c*}, N(R^{4c*})₂, SO₃R^{4c*}, SO₂R^{4*}, SO₂N(R^{4c*})₂, C(O)R^{4c*}; C(O)OR^{4c*}, OC(O)R^{4c*}; C(O)N(R^{4c*})₂, N(R^{4c*})C(O)R^{4c*}, OC(O)N(R^{4c*})₂, N(R^{4c*})C(O)N(R^{4c*})₂, wherein R^{4c*} is in each case independently selected from hydrogen, C₁₋₈alkyl, aryl, C₁₋₈heteroaryl, C₃₋₈cycloalkyl, or C₁₋₈heterocyclyl;
R^{4d} is F, Cl, Br, I, NO₂, CN, R^{4d*}, OR^{4d*}, N(R^{4d*})₂, SO₃R^{4d*}, SO₂R^{4d*}, SO₂N(R^{4d*})₂, C(O)R^{4d*}; C(O)OR^{4d*}, OC(O)R^{4d*}; C(O)N(R^{4d*})₂, N(R^{4d*})C(O)R^{4d*}, OC(O)N(R^{4d*})₂, N(R^{4d*})C(O)N(R^{4d*})₂, wherein R^{4d*} is in each case independently selected from hydrogen, C₁₋₈alkyl, aryl, C₁₋₈heteroaryl, C₃₋₈cycloalkyl, or C₁₋₈heterocyclyl;
X^{5a} is null or CR^{4e}R^{4e′};
X^{5b} is null or CR^{4f}R^{4f′},
R^{4e} is F, Cl, Br, I, NO₂, CN, R^{4e*}, OR^{4e*}, N(R^{4e*})₂, SO₃R^{4e*}, SO₂R^{4e*}, SO₂N(R^{4e*})₂, C(O)R^{4e*}; C(O)OR^{4e*}, OC(O)R^{4e*}; C(O)N(R^{4e*})₂, N(R^{4e*})C(O)R^{4e*}, OC(O)N(R^{4e*})₂, N(R^{4e*})C(O)N(R^{4e*})₂, wherein R^{4e*} is in each case independently selected from hydrogen, C₁₋₈alkyl, aryl, C₁₋₈heteroaryl, C₃₋₈cycloalkyl, or C₁₋₈heterocyclyl;
R^{4e′} is F, Cl, Br, I, NO₂, CN, R^{4e′*}, OR^{4e′*}, N(R^{4e′*})₂, SO₃R^{4e′*}, SO₂R^{4e′*}, SO₂N(R^{4e′*})₂, C(O)R^{4e′*}; C(O)OR^{4e′*}, OC(O)R^{4e′*}; C(O)N(R^{4e′*})₂, N(R^{4e′*})C(O)R^{4e′*}, OC(O)N(R^{4e′*})₂, N(R^{4e′*})C(O)N(R^{4e′*})₂, wherein R^{4e′*} is in each case independently selected from hydrogen, C₁₋₈alkyl, aryl, C₁₋₈heteroaryl, C₃₋₈cycloalkyl, or C₁₋₈heterocyclyl;
wherein R^{4e} and R^{4e′} may together form an oxo or thiooxo;
R^{4f} is F, Cl, Br, I, NO₂, CN, R^{4f*}, OR^{4f*}, N(R^{4f*})₂, SO₃R^{4f*}, SO₂R^{4f*}, SO₂N(R^{4f*})₂, C(O)R^{4f*}; C(O)OR^{4f*}, OC(O)R^{4f*}; C(O)N(R^{4f*})₂, N(R^{4f*}) C(O)R^{4f*}, OC(O)N(R^{4f*})₂, N(R^{4f*})C(O)N(R^{4f*})₂, wherein R^{4f*} is in each case independently selected from hydrogen, C₁₋₈alkyl, aryl, C₁₋₈heteroaryl, C₃₋₈cycloalkyl, or C₁₋₈heterocyclyl;
R^{4f′} is F, Cl, Br, I, NO₂, CN, R^{4f′*}, OR^{4f′*}, N(R^{4f′*})₂, SO₃R^{4f′*}, SO₂R^{4f′*}, SO₂N(R^{4f′*})₂, C(O)R^{4f′*}; C(O)OR^{4f′*}, OC(O)R^{4f′*}; C(O)N(R^{4f′*})₂, N(R^{4f′*})C(O)R^{4f′*}, OC(O)N(R^{4f′*})₂, N(R^{4f′*})C(O)N(R^{4f′*})₂, wherein R^{4f′*} is in each case independently selected from hydrogen, C₁₋₈alkyl, aryl, C₁₋₈heteroaryl, C₃₋₈cycloalkyl, or C₁₋₈heterocyclyl;
wherein R^{4f} and R^{4f′} may together form an oxo or thiooxo;
wherein any two or more of R^{4a}, R^{4a′}, R^{4b}, R^{4b′}, R^{4c}, R^{4d}, R^{4e}, R^{4e′}, R^{4f}, and R^{4f′} may together form a ring;
wherein wavy line 5 represents the point of attachment to A³ and wavy line 6 represents the point of attachment to R^{E}.

145. The compound according to claim 144, wherein A⁴ has the formula: 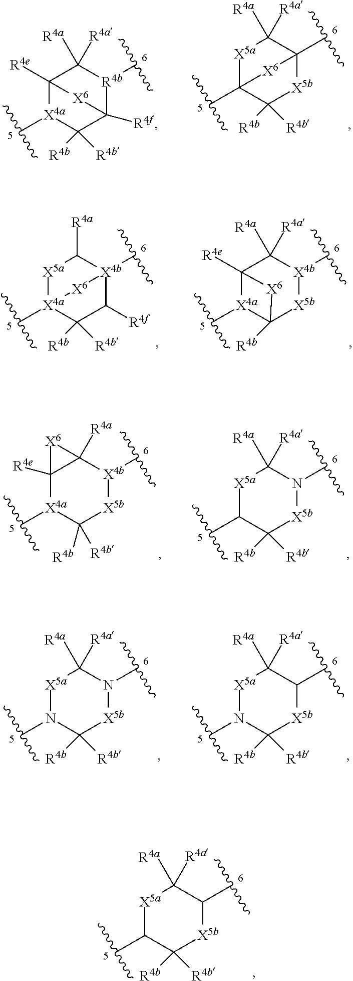
wherein X⁶ is Z², Z²—O, Z²—S, Z²—NRⁿ⁴, Z²—O—Z², Z²—S—Z², Z²—NRⁿ⁴—Z², wherein Z² is in each case null or a group having the formula: 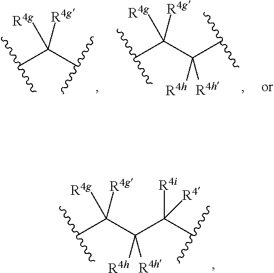
wherein R^{4g} is selected from F, Cl, Br, I, NO₂, CN, R^{4g*}, OR^{4g*}, N(R^{4g*})₂, SO₃R^{4g*}, SO₂R^{4g*}, SO₂N(R^{4g*})₂, C(O)R^{4g*}; C(O)OR^{4g*}, OC(O)R^{4g*}; C(O)N(R^{4g*})₂, N(R^{4g*})C(O)R^{4g*}, OC(O)N(R^{4g*})₂, N(R^{4g*})C(O)N(R^{4g*})₂, wherein R^{4g*} is in each case independently selected from hydrogen, C₁₋₈alkyl, aryl, C₁₋₈heteroaryl, C₃₋₈cycloalkyl, or C₁₋₈heterocyclyl;
wherein R^{4g′} is selected from F, Cl, Br, I, NO₂, CN, R^{4g′*}, OR^{4g′*}, N(R^{4g′*})₂, SO₃R^{4g′*}, SO₂R^{4g′*}, SO₂N(R^{4g′*})₂, C(O)R^{4g′*}; C(O)O R^{4g′*}, OC(O)R^{4g′*}; C(O)N(R^{4g′*})₂, N(R^{4g′*}) C(O)R^{4g′*} OC(O)N(R^{4g′*})₂, N(R^{4g′*})C(O)N(R^{4g′*})₂, wherein R^{4g′*} is in each case independently selected from hydrogen, C₁₋₈alkyl, aryl, C₁₋₈heteroaryl, C₃₋₈cycloalkyl, or C₁₋₈ heterocyclyl;
or wherein R⁴and R^{4g′} together form an oxo, or thiooxo
wherein R^{4h} is selected from F, Cl, Br, I, NO₂, CN, R^{4h*}, OR^{4h*}, N(R^{4h*})₂, SO₃R^{4h*}, SO₂R^{4h*}, SO₂N(R^{4h*})₂, C(O)R^{4h*}; C(O)OR^{4h*}, OC(O)R^{4h*}; C(O)N(R^{4h*})₂, N(R^{4h*})C(O)R^{4h*} OC(O)N(R^{4h*})₂, N(R^{4h*})C(O)N(R^{4h*})₂, wherein R^{4h*} is in each case independently selected from hydrogen, C₁₋₈alkyl, aryl, C₁₋₈heteroaryl, C₃₋₈cycloalkyl, or C, ₈heterocyclyl;
wherein R^{4h′} is selected from F, Cl, Br, I, NO₂, CN, R^{4h′*}, OR^{4h′*}, N(R^{4h′*})₂, SO₃R^{4h′*}, SO₂R^{4h′*}, SO₂N(R^{4h′*})₂, C(O)R^{4h′*}; C(O)OR^{4h′*}, OC(O)R^{4h′*}; C(O)N(R^{4h′*})₂, N(R^{4h′*})C(O)R^{4h′*}, OC(O)N(R^{4h′*})₂, N(R^{4h′*})C(O)N(R^{4h′*})₂, wherein R^{4h′*} is in each case independently selected from hydrogen, C₁₋₈alkyl, aryl, C₁₋₈heteroaryl, C₃₋₈cycloalkyl, or C₁₋₈ heterocyclyl;
or wherein R^{4h} and R^{4h′} together form an oxo, or thiooxo
wherein R⁴ⁱ is selected from F, Cl, Br, I, NO₂, CN, R^{4i*}, OR^{4i*}, N(R^{4i*})₂, SO₃R^{4i*}, SO₂R^{4i*}, SO₂N(R^{4i*})₂, C(O)R^{4i*}; C(O)OR^{4i*}, OC(O)R^{4i*}; C(O)N(R^{4i*})₂, N(R^{4i*})C(O)R^{4i*}, OC(O)N(R^{4i*})₂, N(R^{4i*})C(O)N(R^{4i*})₂, wherein R^{4i*} is in each case independently selected from hydrogen, C₁₋₈alkyl, aryl, C₁₋₈heteroaryl, C₃₋₈cycloalkyl, or C₁₋₈heterocyclyl;
wherein R^{4i′} is selected from F, Cl, Br, I, NO₂, CN, R^{4i′*}, OR^{4i′*}, N(R^{4i′*})₂, SO₃R^{4i′*}, SO₂R^{4i′*}, SO₂N(R^{4i′*})₂, C(O)R^{4i′*}; C(O)OR^{4i′*} OC(O)R^{4i′*}; C(O)N(R^{4i′*})₂, N(R^{4i′*})C(O)R^{4i′*}, OC(O)N(R^{4i′*})₂, N(R^{4i′*})C(O)N(R^{4i′*})₂, wherein R^{4i′*} is in each case independently selected from hydrogen, C₁₋₈alkyl, aryl, C₁₋₈heteroaryl, C₃₋₈cycloalkyl, or C₁₋₈ heterocyclyl;
or wherein R⁴ⁱ and R^{4i′} together form an oxo, or thiooxo.

146. The compound according to claim 145, wherein A⁴ has the formula:

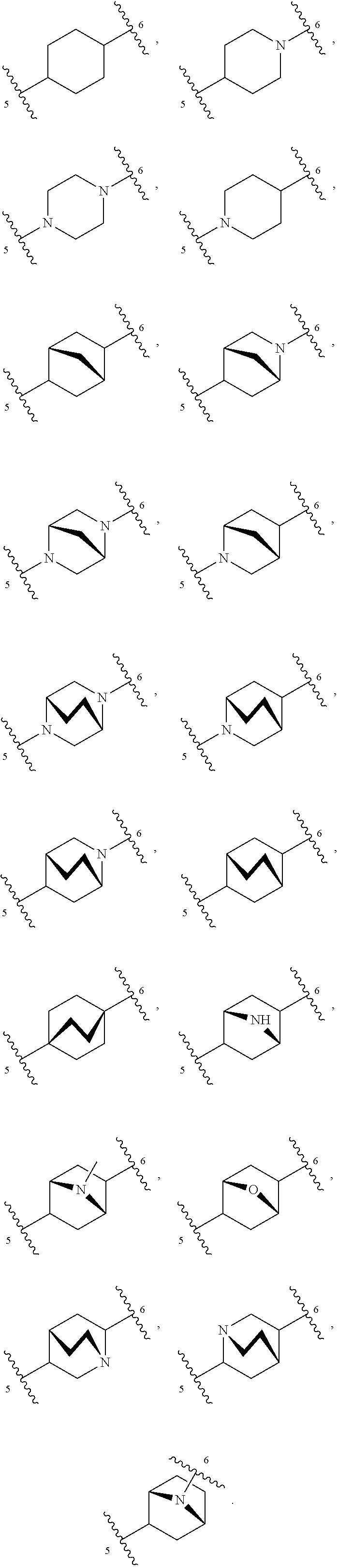

147. The compound according to claim 133, wherein R^{a} has the formula:
—Z³—Z^{c}—Z⁴—Z^{t},
wherein
Z³ is null
Z^{c} is null, —C(═O)—, —C(═O)O—, or —C(═O)NH—
Z⁴ is C₁₋₆alkylene,
Z^{t} is H, F, Cl, Br, I, CN, aryl, C₁₋₈heteroaryl, C₃₋₈cycloalkyl, C₁₋₈heterocyclyl, OR^{za}, N(R^{za})₂, C(═O)OR^{za}, C(═O)NHR^{za*}, wherein R^{za*} is in each case independently selected from hydrogen, C₁₋₈alkyl, aryl, C₁₋₈heteroaryl, C₃₋₈cycloalkyl, or C₁₋₈heterocyclyl

148. The compound of claim 133, A⁴, R^{a} and A³ together form a system having the formula: 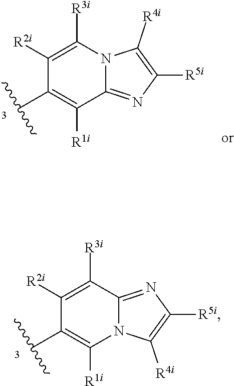
wherein
R¹ⁱ is F, Cl, Br, I, NO₂, CN, R^{1i*}, OR^{1i*}, N(R^{1i*})₂, SO₃R^{1i*}, SO₂R^{1i*}, SO₂N(R^{1i*})₂, C(O)R^{1i*}; C(O)OR^{1i*}, OC(O)R^{1i*}; C(O)N(R^{1i*})₂, N(R^{1i*})C(O)R^{1i*}, OC(O)N(R^{1i*})₂, N(R^{1i*})C(O)N(R^{1i*})₂, wherein R^{1i*} is in each case independently selected from hydrogen, C₁₋₈alkyl, aryl, C₁₋₈heteroaryl, C₃₋₈cycloalkyl, or C₁₋₈heterocyclyl;
R²ⁱ is F, Cl, Br, I, NO₂, CN, R^{2i*}, OR^{2i*}, N(R^{2i*})₂, SO₃R^{2i*}, SO₂R^{2i*}, SO₂N(R^{2i*})₂, C(O)R^{2i*}; C(O)OR^{2i*}, OC(O)R^{2i*}; C(O)N(R^{2i*})₂, N(R^{2i*})C(O)R^{2i*}, OC(O)N(R^{2i*})₂, N(R^{2i*})C(O)N(R^{2i*})₂, wherein R^{2i*} is in each case independently selected from hydrogen, C₁₋₈alkyl, aryl, C₁₋₈heteroaryl, C₃₋₈cycloalkyl, or C₁₋₈heterocyclyl;
R³ⁱ is F, Cl, Br, I, NO₂, CN, R^{3i*}, OR^{3i*}, N(R^{3i*})₂, SO₃R^{3i*}, SO₂R^{3i*}, SO₂N(R^{3i*})₂, C(O)R^{3i*}; C(O)OR^{3i*}, OC(O)R^{3i*}; C(O)N(R^{3i*})₂, N(R^{3i*})C(O)R^{3i*}, OC(O)N(R^{3i*})₂, N(R^{3i*})C(O)N(R^{3i*})₂, wherein R^{3i*} is in each case independently selected from hydrogen, C₁₋₈alkyl, aryl, C₁₋₈heteroaryl, C₃₋₈cycloalkyl, or C₁₋₈heterocyclyl;
R⁴ⁱ is F, Cl, Br, I, NO₂, CN, R^{4i*}, OR^{4i*}, N(R^{4i*})₂, SO₃R^{4i*}, SO₂R^{4i*}, SO₂N(R^{4i*})₂, C(O)R^{4i*}; C(O)OR^{4i*}, OC(O)R^{4i*}; C(O)N(R^{4i*})₂, N(R^{4i*})C(O)R^{4i*}, OC(O)N(R^{4i*})₂, N(R^{4i*})C(O)N(R^{4i*})₂, wherein R^{4i*} is in each case independently selected from hydrogen, C₁₋₈alkyl, aryl, C₁₋₈heteroaryl, C₃₋₈cycloalkyl, or C₁₋₈heterocyclyl;
R⁵ⁱ is F, Cl, Br, I, NO₂, CN, R^{5i*}, OR^{5i*}, N(R^{5i*})₂, SO₃R^{5i*}, SO₂R^{5i*}, SO₂N(R^{5i*})₂, C(O)R^{5i*}; C(O)OR^{5i*}, OC(O)R^{5i*}; C(O)N(R^{5i*})₂, N(R^{5i*})C(O)R^{5i*}, OC(O)N(R^{5i*})₂, N(R^{5i*})C(O)N(R^{5i*})₂, wherein R^{5i*} is in each case independently selected from hydrogen, C₁₋₈alkyl, aryl, C₁₋₈heteroaryl, C₃₋₈cycloalkyl, or C₁₋₈heterocyclyl;
wherein any two or more of R¹ⁱ, R²ⁱ, R³ⁱ, R⁴ⁱ, and R⁵ⁱ can together form a ring.

149. The compound of claim 133, wherein A³, A⁴, and R^{a} together form a system having the formula:

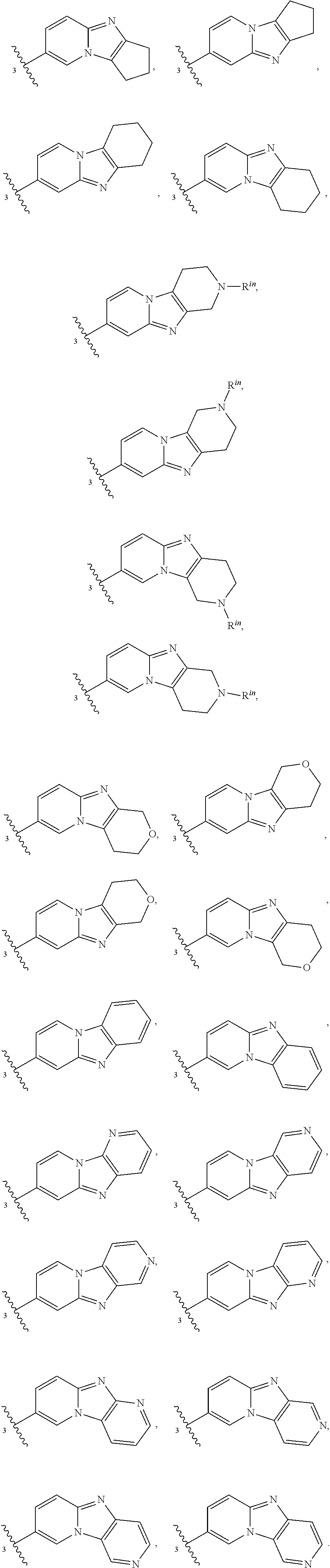

150. The compound of claim 133, wherein A³, A⁴, and R^{a} together form a system having the formula: 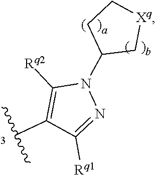
wherein
X^{q} is CH₂, O, or NR^{q3}
R^{q1} is H, F, Cl, Br, C₁₋₃alkyl, OC₁₋₃alkyl,
R^{q2} is H, F, Cl, Br, C₁₋₃alkyl, OC₁₋₃alkyl,
R^{q3} is H, (CH₂)_{c}OH or C(═O)OC₁₋₃alkyl,
a is 0, 1, or 2,
b is 1, 2, or 3, and
c is 1,2, or 3

151. A pharmaceutical composition comprising a compound according to claim 133 and at least one pharmaceutical excipient.

152. A method of treating a proliferative disorder, comprising administering a subject in need thereof a compound according to claim 133.
